SEQUENCE LISTING (38 sequences)

SEQ ID NO: 1 - DNA, 1001 bp, Homo sapiens
gaaagtattgatggtgagtaactttggtaaaagaaggtccaggcatgtggctcatgcctataatcccagcactttgggaggccgaggcaggataattgcttgaggtcaggagttcgagactagcctaggtaacatagtaaaaccctgcccctacaaaaaattaaaatattagccaggtgtgatggtgcacatagtcctgtcatcctagctacttgggaggctgaggcaggaggattgcttgagcccaggaatttgaggctacagtgagctatgattgtgtcactgcactccagcctaggcaaccgagtgagactccatttctaaaagaaattaattaattaataaaaatgaagagaatgctcagcagacttctgaattctttgagtgttcacaagaaaaataaattcatgtatcacatgtcctataaaagaaaaaggggttggggttgcttttgcatataaagcaagctagtttcaaccgaactctctgtccactggagaraagtgcttgccacagaaaactgtttttctggttcactgaggtataattgacaaataacaagtacatatatccaaggtgcacaatgtgatgttttgatataagtatacactgtgaaaggattattacaaccaatttgtagtaattaacttgatgaattaacatattcatcaagtatatagtacattattattaactgtagtcaccatgctgtgctttccatctcagaaaacttagagtattaccctaaagttaactatgaccatttagaagtttgctttaaaacactgcttttcaaattttactgtggaaacaaatcgcttggggatcttgttaaaatttggattctgaatcagtaggtctagagtggggcctgagattctgtctttctgacaatctccccaggtgacaccaatgctgctggtccctgaagcacgcttttggtggcaaggtttccagagagctgaggctcctgtatttctttacaatccagacatcagttt

SEQ ID NO: 2 - DNA, 1001 bp, Homo sapiens
cacctgctactgatacacactataaacgtggaagatgattttcatttttgtagtcatgagcaggatactgtataatgtataattgttggacattaaagaaaacaaactccttcttgtctccttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtrctttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaatagcacgtgtgtcttctttgcagtgtttgctaattttgagtcacatctttttagaaaatcatgagatttggtgtcacagagactggaataaatatagtcaaacttattggtg

SEQ ID NO: 3 - DNA, 1001 bp, Homo sapiens
cacgtctgtcactctaaagcctgttctaagatcacatcccctctggctctccctagatacttctgattatagttggtaatcatgcccattaaatgtctttggtttcttggtacacagagagaaggtaaataatgggttcatgatttaggtaagaatacacaaatatttttcacactataaatctgttttctattcagttaaatattagaccatctggtaatggagaaaatgtttgaaaatccacctagttaacctaaaagtttgcatttcctgcctttggggctttgaatttttaagttacatgtcttcgtagatggtgatcaggataaaactaatatctctcttagatgaatcaaaattcacccccttgggcaagggagggacccagcctttctttaaacatgtggttgcctgagagttgaacaaaactggaatagggagagaatggctttgccatgctgatcatggtattgtggagatttaagaactagtggccaggcraggtggctcacgcctgtaatcccagcattgtgagaggccaaggcaggtggatcacttgaggccaggatttcgagaccagcccggccaacatgatgaccctgtttttactaaaaatacaaaaattagccgagtgtggtggtgtgtgcctgtagtcccaactgaggcatgagacttgcttcaacccaggaggcgggggttgcagtgagccgagattgtgccactgcactccagcctgggtgacagagtgagactctgtctcaaaaaaaaaaaaaaaaaaaactagtcagagctgttgaggtgttgaggcacctgctactgatacacactataaacgtggaagatgattttcatttttgtagtcatgagcaggatactgtataatgtataattgttggacattaaagaaaacaaactccttcttgtctccttaggctcagagccactcagacattgggaagcaagtttgtcaagatgacagagaaccgaggtaatggattcga

SEQ ID NO: 4 - DNA, 1001 bp, Homo sapiens
atagtgagattctgcctctaaaacaaaacaaaataaaagacaaaacaactaattctgttttttaaaaaaaaaaaacacacaacagcaaacttgactataaagattattgctgggcacggtggctcaggcctgtaatcccagcactttgggaggctgaggcgggtggatcacctgaggtcaggagctcaagaccagcctggccaacatggtgaaaccctgtctgtactaataatacaaaaaattagccgggcatggtggtgcatgcctgcaatcccagctactcgggaggatgaggcaggagaatcacttgaacctgggaggtagaggttgcagtgagccgagactgcgccactgcactccagcctgggcaacaagagcaaaactccgtgtccaaaaaaaaaaaaaaaaaaaaagattattatatataatcattcaaggcctgtatgactcagttcccttagaaaaatgtcataatttttatattactgaatattattggcrttatttgtgtagcccacttaagtgaagtcaataacatgattaagtggcatattatcttcatgtcagtcaaacgttatttggattttataagttagggtgagatacaaataagtgaaaatactttttctaatgaataatgatgaatctaaaataggattgacttggctgggcatagtggctcgtgcttgtaaccccaacactttgggaggctgaggcagtaggattacttgaaaccaggagtttgagaccagcctcggcaacaaagggagaactcttctctaataaaaataagaataaaaaattagccaggtgtggcaatgttcacctgtggtcccagctacttgggaagctgaggcaggaggatcgttggagcacaggagttcaagactgcagttagcggtgactgcactccagcctgggcaatagagcaagaccctgtctctaaaaaaaaaataataataaataggactggctcgcatatgtatgcaactattttgtta

SEQ ID NO: 5 - DNA, 1001 bp, Homo sapiens
actgagtcatacaggccttgaatgattatatataataatctttttttttttttttttttttggacacggagttttgctcttgttgcccaggctggagtgcagtggcgcagtctcggctcactgcaacctctacctcccaggttcaagtgattctcctgcctcatcctcccgagtagctgggattgcaggcatgcaccaccatgcccggctaattttttgtattattagtacagacagggtttcaccatgttggccaggctggtcttgagctcctgacctcaggtgatccacccgcctcagcctcccaaagtgctgggattacaggcctgagccaccgtgcccagcaataatctttatagtcaagtttgctgttgtgtgttttttttttttaaaaaacagaattagttgttttgtcttttattttgttttgttttagaggcagaatctcactatgttgcccaggcttattttgcaccactggcctcaagcagtcctcccagytctgccaaattttagacacctggacttggaaacccatgagaagttggccagcgcttccttttgcatttatgcagagcaatggtaaacgtcagcagcaaatttacaatcaatcttattttccagtgtctccagagatttgattgttttgcttatatggcttatggcaatacttacctccaatgtctgatttttcaaaaaaatttcatctaatcctttgtgcaatggtttacatctaattttttttgttctgagatgtagacagctattaagaactgatctttgcacagtaaatttttcctattcttagtcattatccttaggtgaggcaacatggtgccattcagttattcagcaaattctcacatcttccgtgtgccatgcattgttacaggttttgagcctagaaccatgaaaacaaaagacaaaaatctctgcccttgtggactttgtattccagaggagagaaaataaacaaggtaagtaaaatatatagtatggta

SEQ ID NO: 6 - DNA, 1001 bp, Homo sapiens
tgtgttcattatgaccactgggaagatgagtttcacccttcacatgactatatggtgagaacatgtttctcagctgagacaagatttcaggaaagtttgcaagaaccgagagaaaatggaagaaaatgaaatatttgttcttcagagtcaccagttttattatatgcctggactctgtcacttatgtcaataaatttacaaatgcaaaatacacatttaattccagcgtggtagcatacacctgtagtcctagatactcaggagggtgagtatctaggactacaggtgtgtgctaccacgcttgaactcaggagttcaaggccagcctggacaacacagggagaccccctctctaaatgtatatacacacatacacacacacacacacacacacacacattcaaacattggaatcagatgtcctggacaaaatgctcaaatcagctgaacactttggaatgcttaacttttcttttttttagatgtttatttatttatttwttttgttttttattttattattattatactttaagttttagggtacatgtgcgcaatgtgcaggtttgttacatatgtatacatgtgccatgttggtgtgctgcacccattaactcgtcatttagcattaggtatatctccaaatgctatccctcccccctccccccaccccacaacagtccccggagtgtgatgttccccttcctgtgtccatgtgttctcattgttcaattcccacctatgagtgagaacatgcggtgtttggttttttgtccttgtgatagtttgctgaggatgatggtttccagtttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacctacgtgtgcatgtgtctttatagcagcatga

SEQ ID NO: 7 - DNA, 1001 bp, Homo sapiens
cactgacttccacaatggttgaactagtttacggtcccaccaacagtgtaaaagtgttcctatttctccacatcctctccagcacctgttgtttcctgactttttaatgatcaccattctaactggtgtgagatggtatctcattgtggttttgatttgcatttctctgatggccagtgatgatgagcattttttcatgtgttttttggctgcataaatgtcttcttttgagaagtgtctgttcatatccttcgcccactttttgatggggttgtttatttttttcttgtaaatttgtttgagttcattgtagattctggatattagccctttgtcagatgagtaggttgcaaaaattttctcccatttcgtaggttgcttgttcactctgatggtagtttcttttgctggaatgcttaacttttctttcctgcaagaggaagactgggagattgagaggtgtgcccagggtgtcagctcagtgcctggtagaggcaggtmacatgagctttgagccctggcctgtggattgtgttgtgcttggactggcctgttacgccatctgcttgcttctgcctgaaattcctgtaacacaagataaaacctctcattctgcaatttaccaataaaccttataaaatctgaggctaagccttgtgaaatggcaaaccctagaaagccagagagctggcatgcggtagttcagcagtggctgcaggaaatagaaggaaagggagtgaggagacagggtgcaagctggaatgtgatcacaggggagggggatctctggactatgggggaatgacgggcagcttgaggccctgagatgaaagacactccctttaagaaaaacgtctgtgactcagagccacaaggcgtgtcaggagaaagttctgatgaaactgcattccattcctggaggaatacgcattgccattgaagtacaaactctaaaatgatgtaggattaataattaaggcaacactagtagtggatggtgg

SEQ ID NO: 8 - DNA, 1001 bp, Artificial Sequence
  Description of Artificial Sequence Synthetic polynucleotide
ttcttttaaaactaaacatatacagttgtccatcagtatctgcaggggactggttccaggtctccctgcaagtaccaaaatcacagatgctcaggaatctgatataaagcagggtagtacttgcatataacccacacacatcctcctgtatactttaaatcatctctagattacttataatacctagtacaaggtgaagactatgtaaacagttgttatactgtatttaaaaaaataatttccactttcattttagattcaggtggtacatatgcaggtttgttatataagtgtattgcatgatgctgaggtttggagttcaattgatcccatcactcagatagtgagcataattatactgtatacagtttaggaaataataacaagaaaaaagtctatacatattccatacagacatgatcatcctttcctcccaccccttgattttttattgaattcatgaatatttgctgagtccatgaatgtggaacccacggatakggaaggctgactgcacctacattatgacctggcaattccacacctaggttactcacccgggagaaataaaagcatatgtcctcaaagaggcttgttcaaaaatgtccatagctttattcataaataactgaaagctgaaaacaaccaataggagaatgaataaactaactgtggtaaattcagacaatgaaatactacacaataaaaaagggaggaaccggctgggcgcggtggctcacacctgtaatcccagcacattgggaggccgaggtgggtggatcacctggggtcaagagttcgagaccagcctggccaacatggtgaaaccccatctctactaaaaatacaaaaatagtcaggtgtggtggcacgcacctccaatcccagctactcgaaaggctgaggcaggagaatcagcttgaatccaggaggcagaggttgcagtgagctgagattgtgccactgcactccagcctgggtgactctgtctcaaaaaaaaa

SEQ ID NO: 9 - DNA, 1001 bp, Homo sapiens
atgaactaggttttgaactcccccatgcatgacccctgcctgtggggtctgccctagatggataataaataggtcattatcagtctcatttcagtgtccacagtggagagattttattcttccccttgctctggaactggccccttttctcctccaaatcccaatcttggcctagaattttgaactctgcttagaattccaaattgccacatatatatgtcaggagatcagacagagttagctgagcagggaacaaggccgtgcttttcagaagtatgtaggtctgcttcacaagaatatgccattaacaatatggacaaggctcaccataaatttatgagtgaaacaacttattccaactgctctcatgcctggctttatacagtcatctacttgtccttccctgggcccagccaatgctgctccccctttaacaactgcttctgaatgtccctgtggtgtgggccagaaaggagactctcttcttccccaaatccaccygcagtatggcagaaactagaattcatggtcctctcccaacccatgcccacttccttctgccacttaaagaaaacacccataaagggtgggaagagagagcgtaacagcaaggtctgtgcattcccagagatgtgatgcaaggggtgtgggaggcatggcactgcttgactcacgctggagagcgggcacttggcctggctttcagaggaaatgctccttggaatgcggtcggccccggctgcacccacgcctgtgagggaggggcttatgtgtctggcacatcataggtggctcctggggtttgccatgagtctcagcacagcagacctgagaggaaaaaaatacagactgaacgcgtttcttctattctctcacccaacacagaagacttctgtggccgcatgtgtggggttttttttccccacataccaagaagcaaacaattcttctgtggactctagccgagactcttccaattcaattcaattcagacactagct

SEQ ID NO: 10 - DNA, 1001 bp, Homo sapiens
caactgctctcatgcctggctttatacagtcatctacttgtccttccctgggcccagccaatgctgctccccctttaacaactgcttctgaatgtccctgtggtgtgggccagaaaggagactctcttcttccccaaatccacctgcagtatggcagaaactagaattcatggtcctctcccaacccatgcccacttccttctgccacttaaagaaaacacccataaagggtgggaagagagagcgtaacagcaaggtctgtgcattcccagagatgtgatgcaaggggtgtgggaggcatggcactgcttgactcacgctggagagcgggcacttggcctggctttcagaggaaatgctccttggaatgcggtcggccccggctgcacccacgcctgtgagggaggggcttatgtgtctggcacatcataggtggctcctggggtttgccatgagtctcagcacagcagacctgagaggaaaaaaatacagactgaacgygtttcttctattctctcacccaacacagaagacttctgtggccgcatgtgtggggttttttttccccacataccaagaagcaaacaattcttctgtggactctagccgagactcttccaattcaattcaattcagacactagctacctagagatagtgtaagaaggcacaggttgaggactcattccccaagaccacccctcactcctgatgccaactgcaagctccacgttgttttatctgtgcatctgactggctataaatcaggggtcctatggccccctccggtggcccttccttgggtttgattaatatgctagagcagctcacagaactcagggaaacaccgatttattataaaggatattacaaaggataaagattaatagatgcatagggtaaagtatgggggaaggcagaagcagtttccacgtccttcccaagcaccacaccctccaggaacctccacgtggtcagctacccagaaggtctccaaacatg

SEQ ID NO: 11 - DNA, 1001 bp, Homo sapiens
gcagatggggcctttgggaggtgactaggacatgagggtggagcacttgtgagtgggattagtgcccttacagagagactgcagagagctcccttgccccttccaccatgtgaggacacagggagaagatggctgtctgtgaactgggaaacaggccctcatcagacactgagtcagctgaggtattgatcttggacttcccagccttcagaactgtgagaaataaatttctgtttaaaagacacccagtttcaggtatttttgccatagtagcccaaacagactaacagtggatataattttgttgagccagttataaaatccaagtccaagtcaaaactgcaggctgatattagctgggcatggtggtgcatgcctgtagttccagctactccagaggcggaggtgggaggatggcttgggccccagaggtcaaggctgctgtgagtggtgatcacaccaatgcacaccagcctgggggacagagcaagaccctgtctsaaaaacacaaaaatacaaaaaacttgcaggctccaaaactacctgaacttcaacctaccttctttcaatacaatgcccagaacatagtctctctcaatgacccagtcaagcttaaaaaaaaaaaaaagaaaaaaaaagcatcaccacttctctcttcaaaatctagccttgatatatattttgaggggatgcattctgaagtgtgtcagcatgatacatcgtatcatctgcaacttactttcaaatggcttaggaaaaaaatatggtatctgctgtctatttatctacctacctatctatcaagagataagcaaacatgatgaaataataacagttgttaaatccatgtgaggggtatttggctgtttgtgtgctatttttcagcttttctgattgagcttacttttttaaaaaggcaggaaaaagtatgtggccttgactgtgaagacagacaagaaacagctgagcccctcttgtttctcgatacatcaaaaatgcgg

SEQ ID NO: 12 - DNA, 1001 bp, Homo sapiens
acacccataaagggtgggaagagagagcgtaacagcaaggtctgtgcattcccagagatgtgatgcaaggggtgtgggaggcatggcactgcttgactcacgctggagagcgggcacttggcctggctttcagaggaaatgctccttggaatgcggtcggccccggctgcacccacgcctgtgagggaggggcttatgtgtctggcacatcataggtggctcctggggtttgccatgagtctcagcacagcagacctgagaggaaaaaaatacagactgaacgcgtttcttctattctctcacccaacacagaagacttctgtggccgcatgtgtggggttttttttccccacataccaagaagcaaacaattcttctgtggactctagccgagactcttccaattcaattcaattcagacactagctacctagagatagtgtaagaaggcacaggttgaggactcattccccaagaccacccctcactcctgatgccaaytgcaagctccacgttgttttatctgtgcatctgactggctataaatcaggggtcctatggccccctccggtggcccttccttgggtttgattaatatgctagagcagctcacagaactcagggaaacaccgatttattataaaggatattacaaaggataaagattaatagatgcatagggtaaagtatgggggaaggcagaagcagtttccacgtccttcccaagcaccacaccctccaggaacctccacgtggtcagctacccagaaggtctccaaacatggttcttttgggctttgatggaggctttattatgtaggcatgattgattaaactcttggtcattggtgattaactttacttcagcccctcttcccttcccagaggttgggggatgaggctgaaagccccaaccctctaaccatggcttagcctttcccatgaccagtctccgttctgaagctaccgatgggctgccggccatctgtcaactcatcagc

SEQ ID NO: 13 - DNA, 1001 bp, Homo sapiens
gctggtctcaaattcctgacctgaaatgatccgcccgcctcggcctcccaaagtgctgggattacaggtgtaagccaccacgcctggcctgcccatctctttttcacttgagccaaagctatttctaggaaggcagtggcatttcctgagctaaaatcattttcccattcctgagtcacatttcacatggtcccagaggtgaatttagtggattacattttaaaaaacaaacaaaaacctcagagccacacatagaccaggttttgccttcttcctctccaactcccactattccttcctttgcacgtttgctgagccatacggaagtgcatggccaacagagaagaaaaaggttgattagtagtaaagaagccctgctgttgccttgaatgtcagcacgtgcacacacacacacaggtgcgcgcacacacgggcacacacaggtacgcgcacacacacaggtgcaagcacacaggtgcgcgcacacacacagatgcgcgygcacacacaggtacgcgcacacacacttttatacctgtccattgccagtttcttttggttcttcaggattgcccttagtgttcgttttcatctgatgttgcctagaccaaaatcctggaggaaagaacctgatgaactaggttttgaactcccccatgcatgacccctgcctgtggggtctgccctagatggataataaataggtcattatcagtctcatttcagtgtccacagtggagagattttattcttccccttgctctggaactggccccttttctcctccaaatcccaatcttggcctagaattttgaactctgcttagaattccaaattgccacatatatatgtcaggagatcagacagagttagctgagcagggaacaaggccgtgcttttcagaagtatgtaggtctgcttcacaagaatatgccattaacaatatggacaaggctcaccataaatttatgagtgaaacaacttattccaactgctctcat

SEQ ID NO: 14 - DNA, 1001 bp, Homo sapiens
ttttttgttctgagatgtagacagctattaagaactgatctttgcacagtaaatttttcctattcttagtcattatccttaggtgaggcaacatggtgccattcagttattcagcaaattctcacatcttccgtgtgccatgcattgttacaggttttgagcctagaaccatgaaaacaaaagacaaaaatctctgcccttgtggactttgtattccagaggagagaaaataaacaaggtaagtaaaatatatagtatggtagataatgaatggtatgggctgaaggaaaaacataaacaaagacgttaggtagtgctgagagggagggtggtttgcagttgagataaggatgtctccaaggatatggtggcatctgcattaccagagaaacattccaggcagagaaaccagtgagtgcaaaggccctgaggcagaagcatggctggcttgtgtggtaggcatgagtgagctaaggacagaggagctacagaggacgccaragagaaattgagaattgaagggagttgggggatggagctgttggtaccgaagggagacattataaagaccttagcttggctgggcactgtggctcatgcctgtaatcccagcacattgggaagccgaggcaggtggatcacctgaggtcaagagttcgagaccagcctggccaacatggggaaaccccatctctactaaaaatacaaaaattagccgggcgtggtggcgtgcacctgtaatcccagctacttgggaggctgaggcaagagaatcgcttgaacccgggaagtggaggttgcagtgagccaagatcacaccactgcactgcagtctgggcaacaagagtgaaactccatctcaaaaaaaaaaaagaccttagcttttcctctgagagcagaaactttggaggggcttgagtcaagcaatggcgtgatctgcttcgtatcttaacagagtcaccttggctgctcggtcagggcagggggacaagtgtggaggc

SEQ ID NO: 15 - DNA, 1001 bp, Homo sapiens
aggtcaggaatttgagaccagcctggctgacatggtgaaacccggtcgctactaaaaattccaaaaattagccgggcatcgtggcgggcacctgtagtcccagctactcaggaggctgagtctggagaatggcttgaacccagaagatggaggctgcagtgagccaagatcgtgccactgcactccagcctgggcaacagagcgagactccatcttaaaaaaaaaagaaaagaaagaaaaagaaatgggcaagacaaaaccaagtttaagaatgcaagtttattacttatgaaattatagattgcaggaaccagagacttaagtttctccaggcagtagtgtatattatcaggatgaggtaaggaacaccagaccaacagggcagacaggtctgatgaaggaaagggacctgaaggtcattctgaatccagtggagctctaagtaggtcaacttttggcctcctcagaccaacatccctttgtggtgactcaagaccaatktctacctcagggtcaggctggtttggtcaaccacctccagtatggctgacttagttttcaaattcagccacaaggatcacattgaggagtctttttttcagagacagggtcttgccctgttgcccaggctggagtgcaatggtacaatcataactcactgcagcctcgacctcttgggctcaagcaatcctcctgcctcagcctcctgagtagctgggactacagacacacaccaccatgcccagctatttattttatttttgtagaaatgggtcttgctatgttgcccaggctggtcttcaactcctgatcctcccatcttggcctcccaaagtgctgggattacaggcatgagccaccttgcccagctgatcttgaggagtagttctttctttgctctgaagccccatggcttcatttgaatgtatgactaagcccctgttaatggtaaccagtgaaatgaattattattttttttatcatttttatttttttttgag

SEQ ID NO: 16 - DNA, 1001 bp, Homo sapiens
ttgcacttctgtttacaaagcaatcctgccatcaaaagaggaacaaaatcaccacttatcacaccctgtcataaagtaatctgccctggagggaaacccttgtggagaactgctgtgtgtgtgtgtgtgtgtgtgtgtgtgtgtgtgtgtgtgtgttgggggaggggggatggaggaaagggggattctcccaactcttctgcagagtaaatcgctggaacgcgtggtgtcccaaccggcctggaaagaccgagagtaccatgagctgtgaagctggggtgtgacagggatgcccgtccagggctggcaagagtgcagaatggctctcttggatctttggaataggcacatctgcagaccccgctccaatgtttactttcctagcgccttcgaagatactcccaagggcccccaaaatagatcagcaaaaagtgttgggggtggggggagtgaaaaagccagttcttgaagactgtaaggtcccctttcgcatctcagcawctggagtgtgcagggaattcctgaccagtggttttgctccctccaatcccttgcctcccccctcccatgttatgcacttgttcttggagagatggacgttaaagaagcgtcaagcagttctcactgcaaataaatggtgcagaaataagagagagaggatgaaagcctaggaagttataagtgatcctgacccgacccagccaccagggggttatctctttccaggtcctgccttgtgtagagtgaggtgataaacgctttaggcagccaaatccaagcacagctgggtgcctggcggggatggggtgggggtggtcctatgtggtgcctctgcctctggagttacctttaggaaaggtcaagagaactatcctcccctccatgtctgctgaaaaggggctattttgctagtcttgttatcagtaattcaccacttaatataaccaggttttaggtttgtatatgagcgatcctggacatccaataccatccccccagtt

SEQ ID NO: 17 - DNA, 1001 bp, Homo sapiens
tgtcctactgcccttcccagcacctggctttaattggggctgctctgggccactttgccaggaatcctgaagttgatttgtagggaacagggattgagtgaccgggccctacctccgctccccaaaaacaatgtcctgttctcatgtgctggcccacctcctccccaggacctgggtccctacgctgaacactgaggtggcttttgctcagctagtctccaagacagcacgagcctattttgcctatattggtaagagtaatggagctgttcattccagttatctttcactggactgaaaggattggcttaaaaaattactgtaccctactgcgatattgaaaaatatatatttcatcttccaccttgtttccctgtgtacaactcctaaattccttggaatctccaaagtgatgtcttttttgtgtgctgatgagttgacagatggccggcagcccatcggtagcttcagaacggagactggtcatgggaaaggctaagscatggttagagggttggggctttcagcctcatcccccaacctctgggaagggaagaggggctgaagtaaagttaatcaccaatgaccaagagtttaatcaatcatgcctacataataaagcctccatcaaagcccaaaagaaccatgtttggagaccttctgggtagctgaccacgtggaggttcctggagggtgtggtgcttgggaaggacgtggaaactgcttctgccttcccccatactttaccctatgcatctattaatctttatcctttgtaatatcctttataataaatcggtgtttccctgagttctgtgagctgctctagcatattaatcaaacccaaggaagggccaccggagggggccataggacccctgatttatagccagtcagatgcacagataaaacaacgtggagcttgcagttggcatcaggagtgaggggtggtcttggggaatgagtcctcaacctgtgccttcttacactatctctaggta

SEQ ID NO: 18 - DNA, 1001 bp, Homo sapiens
ccttgtggagaactgctgtgtgtgtgtgtgtgtgtgtgtgtgtgtgtgtgtgtgtgtgttgggggaggggggatggaggaaagggggattctcccaactcttctgcagagtaaatcgctggaacgcgtggtgtcccaaccggcctggaaagaccgagagtaccatgagctgtgaagctggggtgtgacagggatgcccgtccagggctggcaagagtgcagaatggctctcttggatctttggaataggcacatctgcagaccccgctccaatgtttactttcctagcgccttcgaagatactcccaagggcccccaaaatagatcagcaaaaagtgttgggggtggggggagtgaaaaagccagttcttgaagactgtaaggtcccctttcgcatctcagcatctggagtgtgcagggaattcctgaccagtggttttgctccctccaatcccttgcctcccccctcccatgttatgcacttgttcttggagagatggasgttaaagaagcgtcaagcagttctcactgcaaataaatggtgcagaaataagagagagaggatgaaagcctaggaagttataagtgatcctgacccgacccagccaccagggggttatctctttccaggtcctgccttgtgtagagtgaggtgataaacgctttaggcagccaaatccaagcacagctgggtgcctggcggggatggggtgggggtggtcctatgtggtgcctctgcctctggagttacctttaggaaaggtcaagagaactatcctcccctccatgtctgctgaaaaggggctattttgctagtcttgttatcagtaattcaccacttaatataaccaggttttaggtttgtatatgagcgatcctggacatccaataccatccccccagttccccagcgccactcctggacattctagacaccagcgaggcttcttctgcagccattcctaatgtagcagatccattttggggggagtctggatgcag

SEQ ID NO: 19 - DNA, 1001 bp, Homo sapiens
taccatgagctgtgaagctggggtgtgacagggatgcccgtccagggctggcaagagtgcagaatggctctcttggatctttggaataggcacatctgcagaccccgctccaatgtttactttcctagcgccttcgaagatactcccaagggcccccaaaatagatcagcaaaaagtgttgggggtggggggagtgaaaaagccagttcttgaagactgtaaggtcccctttcgcatctcagcatctggagtgtgcagggaattcctgaccagtggttttgctccctccaatcccttgcctcccccctcccatgttatgcacttgttcttggagagatggacgttaaagaagcgtcaagcagttctcactgcaaataaatggtgcagaaataagagagagaggatgaaagcctaggaagttataagtgatcctgacccgacccagccaccagggggttatctctttccaggtcctgccttgtgtagagtgaggtgataaaygctttaggcagccaaatccaagcacagctgggtgcctggcggggatggggtgggggtggtcctatgtggtgcctctgcctctggagttacctttaggaaaggtcaagagaactatcctcccctccatgtctgctgaaaaggggctattttgctagtcttgttatcagtaattcaccacttaatataaccaggttttaggtttgtatatgagcgatcctggacatccaataccatccccccagttccccagcgccactcctggacattctagacaccagcgaggcttcttctgcagccattcctaatgtagcagatccattttggggggagtctggatgcaggtgtgtgtgatccagcctgaatttgagactctcagtttctttaacaccagcttgaaaagtctgcaatcactagccctgagagagtactttggttcctaatgggatatcctgagtcagggtggctgaaagagctaccagtttaccttgtacatggcaggc

SEQ ID NO: 20 - DNA, 1001 bp, Homo sapiens
gcgtgagccaccgagcttggccagtgaaatgaattattaaacattattaaccttggtattgatatatcaaaattaatttactaaagagtgttgtggcccacacctgtaatcccagcactttgggaggccaaggtgagaggatttcttgagcccaggagttcaagaccagcctgggcaacaaggccagaccccatccctacaaaaaatttttttaaaaaaatagccaccaggtatggtggtgcacgcctgtggtctcaactgcttgggaggctgaggcaggaggaatgtttgagcccagaaggtcggggctgcagcagtgagctgtgatcacaccactgcattccagcttgggtaacagagtgagatcttttctcaaacaaacaaacaaacaaacaaaaaaaagatttggaatcaatatcctagcaagactctgggttgcaactttgcaaatcttctgctgtgcacgtttgttgttgttgttgagacacagtctcgctctgytgcccaggctggagtgcagtggcacaatcattgctcactgaaacctcgacctcctggactcaagcattcctcccgcgtcagcctcccaagtctctgggactataggcgtgcaccaccacgcctggctaattaaataaaaaattgtgggtgccaggcgcggtggctcacgcctataatcccagcactttgggagggcgaggagggtggatcacgaggtcaagagattgagaccattctggccaacctggtgaaatccagtctctactaaaattacaaaaattagccgggcgtggtggcgcatgcctgtagtcccggctactcgggaggctgaggcaggagaatcacttgaagccgggaggcagaggttgcagtgagccgatattgtaccactgcactccagcctggcgacagagcaagacttcgtttcagaaaaagaaaaaaaattttttttttttgtagaaacagagtctttctatgttgcccaggctgatcgcaaactc

SEQ ID NO: 21 - DNA, 1001 bp, Homo sapiens
ttaaaggcgtgcaccaccatgcatggctaatttttgtatttttagtagagtcaggatttcgacgtgttggccaggctggtctcgaactcctgacctcaggtaatccaccctcctcggcctcccaaagtactaggattacaggcatgagccacattacctggccacatttaagctttttaactaaaagtttattgggagaataaagtggagggcagttaaaatccctctagtggaagaaaagacctggacaatatgagctgtgttcattatgaccactgggaagatgagtttcacccttcacatgactatatggtgagaacatgtttctcagctgagacaagatttcaggaaagtttgcaagaaccgagagaaaatggaagaaaatgaaatatttgttcttcagagtcaccagttttattatatgcctggactctgtcacttatgtcaataaatttacaaatgcaaaatacacatttaattccagcgtggtagcatacaccygtagtcctagatactcaggagggtgagtatctaggactacaggtgtgtgctaccacgcttgaactcaggagttcaaggccagcctggacaacacagggagaccccctctctaaatgtatatacacacatacacacacacacacacacacacacacattcaaacattggaatcagatgtcctggacaaaatgctcaaatcagctgaacactttggaatgcttaacttttcttttttttagatgtttatttatttatttattttgttttttattttattattattatactttaagttttagggtacatgtgcgcaatgtgcaggtttgttacatatgtatacatgtgccatgttggtgtgctgcacccattaactcgtcatttagcattaggtatatctccaaatgctatccctcccccctccccccaccccacaacagtccccggagtgtgatgttccccttcctgtgtccatgtgttctcattgttcaattcccacctat

SEQ ID NO: 22 - DNA, 1001 bp, Homo sapiens
atatctcatgtattgggtatctactatgtgccaagccttttactaggtgctttacacacacatttatattgtgccacttaaaacttatagtaactctaaaagatgatggctgggtgcactggcccacacctgtaatcccagcaagttgggagcccaaggtgggaggatggcttgaggccaggagtttgacaccagcagggagaacatagcaagacctcatctctacaaaattaaaaaaaaaaatacaaaaattagccgagtgtggtggtgcacacctgtagtcccaggtacttgggaggttgaggtgggaggatcacttgagcccaggaggttgaggctgcagtgagctatgattgtaccactgcgctccagctcagataacagagccagaccctatctctaaaatttaaataaataaataaataaataaataaataaataaataaataatgtctattatccccattttgaaaaaaaaaaaaaatctgagagaaggccagrcaccatggctcaaacctgtagacagagacgggcagaaggcttggtcaagagttcgagaccagcctggctaacatggtgaaaacccctgcctctactaaaaatacaaaaattagccaagtatgatggtggcgcctgctatcccagctacttgggaggctgaggcagaattgcttgaacccgggaggcagaggttgcagtgagctgagatcacaccactgcactccagcctgggtgacagagcgagtatccatctcaaaaaaaaaaaaaaaaaaaagcctgagagagaaatcaagcaatatgccccaaattacactactagcaaataacaaaatcaaaattcaatcccaagtctcaattttcttttcaaattcttcacttactatatccgtttcctgttgctgctgtagcacgttactgtgcactttctcacggtgcaggagatgagaagtctgaaatcaaggagtcagcaaggccacagtccctccagaggctccaggaga

SEQ ID NO: 23 - DNA, 1001 bp, Homo sapiens
actaatgcctcttcaaatggagggttttgtttgtttgtttgtttatgggaattttaagtaattttcagtgcctgagaatgttctccataaaacctgtaacaaaacacataataatggttccagtgaaaatagttatctcaaagttggattggatttgaaattctaaataccctatgactagggtatcaaaatttaaggtttggtcaaatgtaactttttaggtgtcttgtgtatggtacaagtttgaaagtgtttatgtgcactacctgttccattcatcatatctacccatatctgtatcacttaaaatgaatacttttaggtttattaaaaagtaacacttcaagcaagcaaatggaaattattttgcagtaactacaaataatagagaagtattaacatagagttgtgggccatgacctagtgggttgtttgtcactgtttattttctgccatttcctaggggtgaattgcatcctgtactgtttacagccttatctmcaacttttgcagagtcaagaatttaaaagcagcagggcttggtggcccatgcctgtaatcccaatattttgggaggccgcagcgggaggatcacttgaggccaggagttccagacctccctgggcaacatgatgagaccacatctctacagaaaaattagctgggcatgctggcatgtgcctgtagtcccagctcctcaagaggctgaggtgggaggatcacttgagcccaggaggtcaaggctgcagtgagctatgatcacaccaatgcactctagcctggggacacagtgagaccctgtctcaaaaaaaaaaaaaagaggaatttaaaagcatttactactatctagtgtactatatacttattattagtttatttttggtctccctcaccagaatgtaggctccttaaaggcagtagtattgcacataatagggactaatatacctttattgaattaattaatgagggccaagatatctcatgtattgggtatctac

SEQ ID NO: 24 - DNA, 1001 bp, Homo sapiens
atttttagtagagatggggtttcaccacgttggccaggctagtctcgatctcctgacctcgtgatcctcccacctcggcctccccaaagtgctgggattacaggcgtgagccaccgagcttggccagtgaaatgaattattaaacattattaaccttggtattgatatatcaaaattaatttactaaagagtgttgtggcccacacctgtaatcccagcactttgggaggccaaggtgagaggatttcttgagcccaggagttcaagaccagcctgggcaacaaggccagaccccatccctacaaaaaatttttttaaaaaaatagccaccaggtatggtggtgcacgcctgtggtctcaactgcttgggaggctgaggcaggaggaatgtttgagcccagaaggtcggggctgcagcagtgagctgtgatcacaccactgcattccagcttgggtaacagagtgagatcttttctcaaacaaacaaacaaacaaacaaamaaaagatttggaatcaatatcctagcaagactctgggttgcaactttgcaaatcttctgctgtgcacgtttgttgttgttgttgagacacagtctcgctctgctgcccaggctggagtgcagtggcacaatcattgctcactgaaacctcgacctcctggactcaagcattcctcccgcgtcagcctcccaagtctctgggactataggcgtgcaccaccacgcctggctaattaaataaaaaattgtgggtgccaggcgcggtggctcacgcctataatcccagcactttgggagggcgaggagggtggatcacgaggtcaagagattgagaccattctggccaacctggtgaaatccagtctctactaaaattacaaaaattagccgggcgtggtggcgcatgcctgtagtcccggctactcgggaggctgaggcaggagaatcacttgaagccgggaggcagaggttgcagtgagccgatattgtaccactgcactc

SEQ ID NO: 25 - DNA, 1001 bp, Homo sapiens
agttgtgggccatgacctagtgggttgtttgtcactgtttattttctgccatttcctaggggtgaattgcatcctgtactgtttacagccttatctccaacttttgcagagtcaagaatttaaaagcagcagggcttggtggcccatgcctgtaatcccaatattttgggaggccgcagcgggaggatcacttgaggccaggagttccagacctccctgggcaacatgatgagaccacatctctacagaaaaattagctgggcatgctggcatgtgcctgtagtcccagctcctcaagaggctgaggtgggaggatcacttgagcccaggaggtcaaggctgcagtgagctatgatcacaccaatgcactctagcctggggacacagtgagaccctgtctcaaaaaaaaaaaaaagaggaatttaaaagcatttactactatctagtgtactatatacttattattagtttatttttggtctccctcaccagaatgtaggytccttaaaggcagtagtattgcacataatagggactaatatacctttattgaattaattaatgagggccaagatatctcatgtattgggtatctactatgtgccaagccttttactaggtgctttacacacacatttatattgtgccacttaaaacttatagtaactctaaaagatgatggctgggtgcactggcccacacctgtaatcccagcaagttgggagcccaaggtgggaggatggcttgaggccaggagtttgacaccagcagggagaacatagcaagacctcatctctacaaaattaaaaaaaaaaatacaaaaattagccgagtgtggtggtgcacacctgtagtcccaggtacttgggaggttgaggtgggaggatcacttgagcccaggaggttgaggctgcagtgagctatgattgtaccactgcgctccagctcagataacagagccagaccctatctctaaaatttaaataaataaataaataaat

SEQ ID NO: 26 - DNA, 1001 bp, Homo sapiens
atgagaactcactcactatcatgaggacagcaagggggaaattctcccccatgagccaatcacctcccaccaggtccctcccccaacattaggaattacaatttgcatgagatttgtgtggccacacggagccaaaccatatcacattggtcaacctatatagtaatgtttttcttaatctaaatgtatactaagttagtgttttatcgatttaaaaatacatcttgaaaaggattttgcaatttactttttttttttttttgtgagacagagtctcactcttgtcccccaggctggagtgtagtagcgtgatcttggctcgctgcaatctctgcctcccaggttcaagcaattctcctgcctcagcctcctgagtagcttggattacaggcgcctgccactactcccggctaattttttggtatttttagtagagacagggtttcaccatgttggccaggctggtttcaaactcctgacctcaagtgatccgcccacctyggcttcccaaagtgctaggattacagacgtgagccaccatgcccagcccacaatttcttttaaaactaaacatatacagttgtccatcagtatctgcaggggactggttccaggtctccctgcaagtaccaaaatcacagatgctcaggaatctgatataaagcagggtagtacttgcatataacccacacacatcctcctgtatactttaaatcatctctagattacttataatacctagtacaaggtgaagactatgtaaacagttgttatactgtatttaaaaaaataatttccactttcattttagattcaggtggtacatatgcaggtttgttatataagtgtattgcatgatgctgaggtttggagttcaattgatcccatcactcagatagtgagcataattatactgtatacagtttaggaaataataacaagaaaaaagtctatacatattccatacagacatgatcatcctttcctcccaccccttgatt

SEQ ID NO: 27 - DNA, 1001 bp, Homo sapiens
cagacagggtcttgctctgtcccccaggctggtgtgcattggtgtgatcaccactcacagcagccttgacctctggggcccaagccatcctcccacctccgcctctggagtagctggaactacaggcatgcaccaccatgcccagctaatatcagcctgcagttttgacttggacttggattttataactggctcaacaaaattatatccactgttagtctgtttgggctactatggcaaaaatacctgaaactgggtgtcttttaaacagaaatttatttctcacagttctgaaggctgggaagtccaagatcaatacctcagctgactcagtgtctgatgagggcctgtttcccagttcacagacagccatcttctccctgtgtcctcacatggtggaaggggcaagggagctctctgcagtctctctgtaagggcactaatcccactcacaagtgctccaccctcatgtcctagtcacctcccaaaggccccatctgytaataccatcaccttggggattagaataccagtgtatgaatttggaggggagataagcattcagtccattgcacccttatttccaaggcccagggataacgctgagctcctctgtgggtgaagcacattcagctataaaacagtatcttaagattttcttctcgagttagatttggtacgtagataacgacctttaactatttgcatctatgcagcttttacttccacctcctcaacccactgtctacaattctcacatagaattaagaataattttgcatagcagataattggctgagcatgcggtattcttttgacccattcaagtgaataaaatactgtataggaacactgtcacaatttaaatgaaattaaatttcttgccccttttcctcccccgaccatttagtctttgggtagcaacagagatcactaacatgatataacaattaatgtagttttgtttcagggcattaatttgatacaaattgtaattctgt

SEQ ID NO: 28 - DNA, 1001 bp, Homo sapiens
gattagaataccagtgtatgaatttggaggggagataagcattcagtccattgcacccttatttccaaggcccagggataacgctgagctcctctgtgggtgaagcacattcagctataaaacagtatcttaagattttcttctcgagttagatttggtacgtagataacgacctttaactatttgcatctatgcagcttttacttccacctcctcaacccactgtctacaattctcacatagaattaagaataattttgcatagcagataattggctgagcatgcggtattcttttgacccattcaagtgaataaaatactgtataggaacactgtcacaatttaaatgaaattaaatttcttgccccttttcctcccccgaccatttagtctttgggtagcaacagagatcactaacatgatataacaattaatgtagttttgtttcagggcattaatttgatacaaattgtaattctgttctcatcagttctgtaaaytgctttactgtaattacaccaagtatttgatcaaatattgccgatatttccatctgtttagtgtatggtattatgatttcaaaattgattctagatttagtccaataatttttagaatgtctctttctatataaaagtcaatgcagaaataataatattttgagataaaaaataaaggcatcttctagttaatataacagattttagtcacatttatattctttaatgttcagtgtatgtatccactacatgagaaactcaagacacaaacaaaatggttatatttactgcaacactaagcaatatggaacattaaaaagaatatgtattcataaacaagacaaaagatggtatagcaacataaaatttacaagaacattatagctggactgtataggaagagcttgactgatttctttccacaatgcattttcatcaaaactcatacatattcagagatacaaatagcataccagtgaattcaatgaactgccactgaa

SEQ ID NO: 29 - DNA, 1001 bp, Homo sapiens
ccagtgtatgaatttggaggggagataagcattcagtccattgcacccttatttccaaggcccagggataacgctgagctcctctgtgggtgaagcacattcagctataaaacagtatcttaagattttcttctcgagttagatttggtacgtagataacgacctttaactatttgcatctatgcagcttttacttccacctcctcaacccactgtctacaattctcacatagaattaagaataattttgcatagcagataattggctgagcatgcggtattcttttgacccattcaagtgaataaaatactgtataggaacactgtcacaatttaaatgaaattaaatttcttgccccttttcctcccccgaccatttagtctttgggtagcaacagagatcactaacatgatataacaattaatgtagttttgtttcagggcattaatttgatacaaattgtaattctgttctcatcagttctgtaaattgctttactrtaattacaccaagtatttgatcaaatattgccgatatttccatctgtttagtgtatggtattatgatttcaaaattgattctagatttagtccaataatttttagaatgtctctttctatataaaagtcaatgcagaaataataatattttgagataaaaaataaaggcatcttctagttaatataacagattttagtcacatttatattctttaatgttcagtgtatgtatccactacatgagaaactcaagacacaaacaaaatggttatatttactgcaacactaagcaatatggaacattaaaaagaatatgtattcataaacaagacaaaagatggtatagcaacataaaatttacaagaacattatagctggactgtataggaagagcttgactgatttctttccacaatgcattttcatcaaaactcatacatattcagagatacaaatagcataccagtgaattcaatgaactgccactgaaaccaaaacat

SEQ ID NO: 30 - DNA, 1001 bp, Homo sapiens
agaattgtgttttactgtagtataccataccttatatgtttaatagaacatatctcatctatattttgtaattcatttttataaaactgcctgtaaatagaaaaattttattatctctttcatgtctacagcttctacatttatgtcccctcttactttttttttttttggagagacagtgtctcactatgttgcctagaccagtttcaaactcctgggctcaagtgatccttctgcctcagcctcccaaagcgttggaactacaggtgtgagccagcccgcctggcccctcttacattctttgttgttgttgttttgttgttgttgtctttgttgttttttgaagcagagtctccctctgtggcccaggctggagtgcagtggcttgatcgtggctcactgcaacctccgcctcccaggttcaagccattctcctgcctcagcttcccgagtagctaggactgtaggcatatgccaccacgcccagctaattttttttayaattttagtagagatgggttttcaccatgttggccaggctggcctcaagctcctgacctccagtgatcttcctgccttggcctcccaaaatgctgggattacaggcatgagccactgggccgagcccccttacattcttaatatagtttatttgtgtcctttcttttttcactttttcactcttgcttacaggtttttcaattgtgttaggctttttaaagaactgcttgtctttgtaatgctctatattataaattttagttgtatttatttacttctgctcttacattaatttttcatttttgatctttgaatttattttgctgttgttttctaattctaatttggatgactagctaattaaaaaaaatgttttcccacttcttaaatgtcagcatttataggcttttgcgctatacatttcactcaaagtagattttgtgaaatgccacaatatttggcatgtggcattttcacaattgtcaattcaaaatgttttc

SEQ ID NO: 31 - DNA, 1001 bp, Homo sapiens
agctgtgatattgtgaatataagaattgtacaattttcctttgaaatgtgctgctgagaagaataaaaatgaagttttctctctggaaatggctggaaacaaacgtcaaaacctgacaatttacacacacagttctctttctgacttgactcacccctttgagaattactttttaaaacaccaaagttataggatactaagttaattgtggtctttctaattctgaaaaactggttttcatttgcctcaagaacttttaaggcaaagttgtctaagatacttccttgaacaaagactcagacaaaagcatttgactgcttttaatttctcagcatttttacattttaaaactatagcttgaatgaaactcaagtgtcctgaataaagaataaatacttaaaaattgtttaaatacatattttctcctttcattgttggagcattcaagcaaagattgtgtaaaattcaggttaagtaaaatgtaaaaaatacatatccagwttacattacatattctttttgtgtacaaatatttataccaataaaaaccccctaagatatttatatctttaacatctatttttcttttacctttactactagaaagagaagctaacaaaggaaagcctcttcaaaaaatgggatttccttggccttagcagttctggtgtgttccactgccaacactaggtaggaaaaatctgacttgtgatgttgtgattaaatagcggcctggctcaaactgcccaaagagagggaattcactatgagttccacagttttatcttgaggaagaaactagcagaaacacagaattttagagccttagagctctcattacaaaatggccctcactataaagtggcatatactgaacccagcttagatgtgtgtactgagcccagtacaaatgtatatagtgaacccagagctctcattacaaaatgcctctcactataaagtggcatatactgaacccagcttaggtgtgtgtactgagc

SEQ ID NO: 32 - DNA, 1001 bp, Homo sapiens
ttgagcccaggagttcaagaccagcctgggcaacaaggccagaccccatccctacaaaaaatttttttaaaaaaatagccaccaggtatggtggtgcacgcctgtggtctcaactgcttgggaggctgaggcaggaggaatgtttgagcccagaaggtcggggctgcagcagtgagctgtgatcacaccactgcattccagcttgggtaacagagtgagatcttttctcaaacaaacaaacaaacaaacaaaaaaaagatttggaatcaatatcctagcaagactctgggttgcaactttgcaaatcttctgctgtgcacgtttgttgttgttgttgagacacagtctcgctctgctgcccaggctggagtgcagtggcacaatcattgctcactgaaacctcgacctcctggactcaagcattcctcccgcgtcagcctcccaagtctctgggactataggcgtgcaccaccacgcctggctaattaaataaaaaattgygggtgccaggcgcggtggctcacgcctataatcccagcactttgggagggcgaggagggtggatcacgaggtcaagagattgagaccattctggccaacctggtgaaatccagtctctactaaaattacaaaaattagccgggcgtggtggcgcatgcctgtagtcccggctactcgggaggctgaggcaggagaatcacttgaagccgggaggcagaggttgcagtgagccgatattgtaccactgcactccagcctggcgacagagcaagacttcgtttcagaaaaagaaaaaaaattttttttttttgtagaaacagagtctttctatgttgcccaggctgatcgcaaactcctgggctcaagggatcctctcacctcccaaagtgctgggattacaggcctgagccaccttccccagccctatgcacattttcacaaagatattttgaaccctagcagtgggaaaggatactttttgactggtgacatcccagagc

SEQ ID NO: 33 - DNA, 1001 bp, Homo sapiens
taatgtaacaggtaggaaacactcaatattcgggaatgctgatgagtttcctgagtagcatgcgactggaatgggaagaacaggaatctggtatcagcctagccacaggctggctgttatgtccttgggcaagtttcatcttgtctctgggttgtatttcctgacctgtaatgcaagagtatcagtcccaaccatctctgaggccctttccagctctcagagactctggaatcagattcatacatctgtcagctgagtttccaaacaacacagcctggaaacaacaattctaaaaataaaacatacaactaagaaaccagtcacaagactagaaaacatcatcacatgtattctgtcactggtaacaaaatagttgcatacatatgcgagccagtcctatttattattattttttttttagagacagggtcttgctctattgcccaggctggagtgcagtcaccgctaactgcagtcttgaactcctgtgctccaacgatyctcctgcctcagcttcccaagtagctgggaccacaggtgaacattgccacacctggctaattttttattcttatttttattagagaagagttctccctttgttgccgaggctggtctcaaactcctggtttcaagtaatcctactgcctcagcctcccaaagtgttggggttacaagcacgagccactatgcccagccaagtcaatcctattttagattcatcattattcattagaaaaagtattttcacttatttgtatctcaccctaacttataaaatccaaataacgtttgactgacatgaagataatatgccacttaatcatgttattgacttcacttaagtgggctacacaaataacgccaataatattcagtaatataaaaattatgacatttttctaagggaactgagtcatacaggccttgaatgattatatataataatctttttttttttttttttttttggacacggagttttgctcttgttgcccagg

SEQ ID NO: 34 - DNA, 1001 bp, Homo sapiens
tcccatcactcagatagtgagcataattatactgtatacagtttaggaaataataacaagaaaaaagtctatacatattccatacagacatgatcatcctttcctcccaccccttgattttttattgaattcatgaatatttgctgagtccatgaatgtggaacccacggatagggaaggctgactgcacctacattatgacctggcaattccacacctaggttactcacccgggagaaataaaagcatatgtcctcaaagaggcttgttcaaaaatgtccatagctttattcataaataactgaaagctgaaaacaaccaataggagaatgaataaactaactgtggtaaattcagacaatgaaatactacacaataaaaaagggaggaaccggctgggcgcggtggctcacacctgtaatcccagcacattgggaggccgaggtgggtggatcacctggggtcaagagttcgagaccagcctggccaacatggtgaaamcccatctctactaaaaatacaaaaatagtcaggtgtggtggcacgcacctccaatcccagctactcgaaaggctgaggcaggagaatcagcttgaatccaggaggcagaggttgcagtgagctgagattgtgccactgcactccagcctgggtgactctgtctcaaaaaaaaagggggggtggggggaggaaccattgatacatacaacatcatgatgaattccaaaaatgttgtactgaatggaagaagccttacacaagaaagcacatactgtttatatatttatcatcctagaacaagcaaaactaatctatggactaatctaaggtgggggcaggggaatccagagaaagggttacctttgggagttgggccatggggttggggaccagctgtgcaggggagctttcagggcagtcttaaggtactggatctcaccaggggtttgccttgcttgtatacatggccttccttgcaacttgctgaatggcacacttat

SEQ ID NO: 35 - DNA, 1001 bp, Homo sapiens
  modified_base (501)..(501): a, c, t or g
ttgcaaacatttattttctcacagttccggaggctggaagtccaagatggagctgctgtgagggttggtttccggtgaggcctttcttcctagctcgtagacagccaccttctctctgtgtcctcacatggcttttcttttgtgtccatgccgagagagaggactctctgaggactctccctcttcttgtaaggacaccagtcctatcagactagggccccactcttatgacctcatttaatttaattatgtctgtaaaggcccctgctccaaatatagtcacattgaggattatggcttcataatcctgtgactctggggagaggacacatttcagtccataacaaagcccttagtgtgttttagtgctcaaaactgttcattcatacctcggttattccattattattgcctacgatattaccacttcagggtttttgttattttttacaatatagagcacaacgtataataaactacacatacgaattctcattgagnaattacagaaaatatatctatcgcgtctaacaaggtttaattagcatcttggaaaaaaaaaaaaacacctacgtttttaaggaaaaagttggccaatactgccatctgttggaattttggtcaaagctcatgtgttggactttactcattctttgtcaatatcttttctttctttctttctttctttttttctgagacggagccttgctctgttacccaggctggagtgtggtggcgcgatctcggctcactgcaacctccgcctcctaggttcaagcgattctcctgccttggcctcctgagtagctggaattacaggcacgcgccaccacgcccggctaatttttgtatttttagtagagacggagtttcaccatgttggtcaggctggtttcgaactcctgacctcgtgatccacccacctcggcctcccaaagtgctggaattacaggcgtgagccaccgcgcccggccctttgtcaacatcttatatgttgctgt

SEQ ID NO: 36 - DNA, 401 bp, Homo sapiens
gtgagagacagagtcacaaagagaaagagacagtgaggggccagaacgactctcttttctccgattgtcaatgcccagtgggagccgggagcccaacaggcccagcccatcagattcggcccctccgggccccaaatccgctcgccccacccgagatccaggcctccagccacttgcctaactgtgagcccgcaagagccmggcccgcggctccctccttcctcctcctgcggcagtctcgcggctttcaaaccttagtcgaacccacagaaggcccagtcccaggccaaacctactcaacaggcaccttctcacggcctaggaattctgcagcgaaattcactggaatttgaggagaaaacccaaagactgctccgaaaggactcccccagtcttcagcc

SEQ ID NO: 37 - DNA, 1001 bp, Homo sapiens
ctatggaagtttctcaaaaaattaaaaatagaactaccatgtgatccagaaatcccactgctgggtatttatccaaaggaaaaaaaatcaatatatcaaagggagacctgcactcccatgtttattgcagcactattcacaatagccaagatacagtatcaatctaagtgtccatcaacagatgaatggataaagcaaatgtgatacacacacacacacacacacacacacacacacacaatggaatactattaagccgtaaaaaagaatgaaattctatcatttgcaggaacatgtatggaattgaagggcatcttgttaagtaaaatcagccaggcaccgaaagacaaatattgcatattcttactcatatgtgggagctaaaaagatggatctcatggaggtagagaaaagaatggtagctactagaggctatgaagggtgtgtgggatgaagagaggttggttaataggtacagacatatagttagacggaataagygctagtattcagcctcaaagtagggtgactatagttaacaaaaacatattgagtatctcaaaatagccagaagagaaaatttgaaatgttcctagctcaaagaaatgatacatgttcaaggcgatggatatcctaaataccctgatttgatcattacacattctatggctgtgtcaagatatcacagataccccataaatatgtgtaattattatgtatcaaaaactttatataaaaaacattaatttgctgtatttttgattctacaattgggcagcactttattccataaaatagaatgagtgttctgatgagccaagaagaggaggttggttttacagacagaaaagggctgtggaaagcagaaaaaaacaaacaaaaaaaatgtggattggtcatttcaaagtttcttttcatgtaaaggttaaagcagaggggactttcttgtcctgctggcactggatctaggtcagtgttggagacgatgtctgggactcagg

SEQ ID NO: 38 - DNA, 121 bp, Homo sapiens
tttgggggctgagtgttcttccaatcctaaaaacaactctctgtggccaaattgctccagrcctccaacatatccaacatagctatatgtgaatagagtcatcagcttctgctgttcgtat

(19) United States
(12) Patent Application Publication
(10) Pub. No.: US 2016/0251722 A1
(43) Pub. Date: Sep. 1, 2016

(54) BRCA2-SPECIFIC MODIFIER LOCUS RELATED TO BREAST CANCER RISK

(71) Applicant: Memorial Sloan-Kettering Cancer Center, New York, NY (US)

(72) Inventors: KENNETH OFFIT, New York, NY (US); Mia M. Gaudet, Atlanta, GA (US); Karoline B. Kuchenbaecker, Cambridge (GB); Vijai Joseph, New York, NY (US); Robert J. Klein, New York, NY (US); Antonis C. Antoniou, Cambridge (GB)

(73) Assignee: Memorial Sloan-Kettering Cancer Center, New York, NY (US)

(21) Appl. No.: 14/866,340

(22) Filed: Sep. 25, 2015

Related U.S. Application Data
(63) Continuation of application No. PCT/US2014/032038, filed on Mar. 27, 2014.
(60) Provisional application No. 61/805,783, filed on Mar. 27, 2013.

(51) Int. Cl. C12Q 1/68

(52) U.S. Cl. CPC C12Q 1/6886; C12Q 2600/156; C12Q 2600/118; C12Q 2600/106

(57) ABSTRACT

The present invention relates to novel SNP biomarkers and to panels of biomarkers that may be used in assessing the risk that a patient carrying a BRCA2 mutation will develop breast cancer. It is based, at least in part, on the identification of a SNP located at 6p24 in the human genome which is associated with breast cancer risk in subjects carrying the BRCA2 mutation but not in the general population. In specific non-limiting embodiments, a SNP from the 6p24 region may be used alone or together with one or more breast cancer risk biomarker to evaluate the likelihood that a subject will develop breast cancer.

### CROSS-REFERENCE TO RELATED APPLICATIONS

This application is a continuation of International Patent Application No. PCT/US14/032038, filed Mar. 27, 2014, which claims priority to U.S. Provisional Patent Application Ser. No. 61/805,783, filed Mar. 27, 2013, to both of which priority is claimed and the contents of both of which are incorporated herein in their entireties.

### SEQUENCE LISTING

This application contains a Sequence Listing which has been submitted electronically in ASCII format and is hereby incorporated by reference in its entirety. Said ASCII copy, created on Sep. 25, 2015, is named 072734.0156CON_SL.txt and is 51,560 bytes in size.

### 1. INTRODUCTION

The present invention relates to novel single nucleotide polymorphism (“SNP”) biomarkers and to panels of biomarkers that may be used in assessing the risk that a patient carrying a BRCA2 mutation will develop breast cancer. In particular non-limiting embodiments, the invention relates to biomarkers (minor alleles) in the human chromosome 6p24 region as indicators of decreased risk of developing breast cancer.

### 2. BACKGROUND OF THE INVENTION

The lifetime risk of breast cancer associated with carrying a BRCA2 mutation varies from 40 to 84% [1]. A genome-wide association study (“GWAS”) of BRCA2 mutation carriers from the Consortium of Investigators of Modifiers of BRCA1/2 (CIMBA) was performed in order to determine whether common genetic variants modify breast cancer risk for BRCA2 mutation carriers [2]. Using the Affymetrix 6.0 platform, the discovery stage results were based on 899 young (<40 years) affected and 804 unaffected carriers of European ancestry. In a rapid replication stage wherein 85 discovery stage SNPs with the smallest P-values were genotyped in 2,486 additional BRCA2 mutation carriers, only published loci associated with breast cancer risk in the general population, including FGFR2 (10q26; rs2981575; P=1.2×10-8), were associated with breast cancer risk at the genome-wide significance level among BRCA2 mutation carriers. Two other loci, in ZNF365 (rs16917302) on 10q21 and a locus on 20q13 (rs311499), were also associated with breast cancer risk in BRCA2 mutation carriers with P-values <10-4 (P=3.8×10-5 and 6.6×10-5, respectively). A nearby SNP in ZNF365 was also associated with breast cancer risk in a study of unselected cases [3] and in a study of mammographic density [4]. Additional follow-up replicated the findings for rs16917302, but not rs311499 [5] in a larger set of BRCA2 mutation carriers. There remained a need to identify additional breast cancer risk modifying loci for BRCA2 mutation carriers.

### 3. SUMMARY OF THE INVENTION

The present invention relates to novel SNP biomarkers and to panels of biomarkers that may be used in assessing the risk that a patient carrying a BRCA2 mutation will develop breast cancer. It is based, at least in part, on the identification of a SNP located at 6p24 in the human genome which is associated with breast cancer risk in subjects carrying the BRCA2 mutation but not in the general population. In specific non-limiting embodiments, a SNP from the 6p24 region may be used alone or together with one or more breast cancer risk biomarker to evaluate the likelihood that a subject will develop breast cancer.

### 4. BRIEF DESCRIPTION OF THE FIGURES

FIG. 1. Associations between SNPs in the region surrounding rs9348512 on chromosome 6 and breast cancer risk for BRCA2 mutation carriers. Results based on imputed and observed genotypes. The blue spikes indicate the recombination rate at each position. Genotyped SNPs are represented by diamonds and imputed SNPs are represented by squares. Color saturation indicates the degree of correlation with the SNP rs9348512.

FIG. 2. Predicted breast cancer risks for BRCA2 mutation carriers by the combined SNP profile distributions of the known breast cancer susceptibility loci at FGFR2, TOX3, 12p11, 5q11, CDKN2A/B, LSP1, 8q24, ESR1, ZNF365, 3p24, 12q24, 5p12, 11q13 and the newly identified BRCA2 modifier locus at 6p24. The figure shows the risks at the 5th and 95th percentiles of the combined genotyped distribution as well as minimum, maximum and average risks.

FIG. 3A-C. Cluster plots for SNPs (A) rs9348512, (B) rs619373, and (C) rs184577.

FIG. 4A-B. Multidimensional scaling plots of the top two principal components of genomic ancestry of all eligible BRCA2 iCOGS samples plotted with the HapMap CEU, ASI, and YRI samples: (A) samples from Finland and BRCA2 6174delT carriers highlighted, and (B) samples, indicated in red, with >19% non-European ancestry were excluded.

FIG. 5A-C. Quantile-quantile plot comparing expected and observed distributions of P-values. Results displayed (A) for the complete sample, (B) after excluding samples from the GWAS discovery stage, and (C) for the complete sample and a set of SNPs from the iCOGS array that were selected independent from the results of the BRCA2 mutation carriers.

FIG. 6. Manhattan plot of P-values by chromosomal position for 18,086 SNPs selected on the basis of a previously published genome-wide association study of BRCA2 mutation carriers. Breast cancer associations results based on 4,330 breast cancer cases and 3,881 unaffected BRCA2 carriers.

FIG. 7. Forest plot of the country-specific, per-allele hazard ratios (HR) and 95% confidence intervals for the association between breast cancer and rs9348512 genotypes.

FIG. 8A-B. Forest plot of the country-specific, per-allele hazard ratios (HR) and 95% confidence intervals for the association with breast cancer for (A) rs619373 and (B) rs184577 genotypes.

### 5. DETAILED DESCRIPTION OF THE INVENTION

For clarity of disclosure and not by way of limitation the detailed description of the invention is divided into the following subsections:

(i) the BRCA2 Modifier Locus and its biomarkers;

(ii) risk assessment biomarker panels;

(iii) kits;

(iv) prognostic methods; and

(v) methods of treatment.

By way of introduction, the present invention relates to biomarkers which are allelic variations, allelic variants and/or single nucleotide polymorphisms. The biomarkers may be represented as nucleic acid molecules, for example SNPs, or may be represented as protein expression product of said nucleic acid.

The term “allelic variation” refers to the presence, in a population, of different forms of the same gene characterized by differences in their nucleotide sequences (sequences in genomic DNA). The variation may be in the form of one or more substitution, insertion, or deletion of a nucleotide. Different alleles may be functionally the same, or may be functionally different. In one subset of allelic variations, a single nucleotide is different between alleles and is referred to as a Single Nucleotide Polymorphism (“SNP”). Allelic variation in a known sequence may be identified by standard sequencing techniques. A “variation” or “variant,” as those terms are used herein, is relative to the ancestral gene found in the majority of the population. Unless specified otherwise, the presence of a SNP means that at the single nucleotide position for which alleles have been identified, the nucleotide present is the variant nucleotide (also referred to as the “minor allele”), not the nucleotide found in the majority of the population (also referred to as the “major allele”). The variation (variant) is comprised of a substituted nucleotide or nucleotides or an insertion or deletion of a nucleotide or nucleotides. Herein, generally the ancestral nucleotide (major allele) is listed first and the variation (variant, minor allele) nucleotide is listed second (for example, in A/G A is the ancestral nucleotide and G is the variation (variant) nucleotide). If there is an insertion, the ancestral nucleotide is represented by a hyphen (e.g., -/G). If there is a deletion, the variation (variant) nucleotide is represented by a hyphen (e.g., G/-). Numerous allelic variations (variants), captured in SNPs, of genes are known in the art and catalogued (for example, in the National Center for Biotechnology Information “Entrez SNP”). Allelic variations that are not SNPs include deletions or insertions or substitutions of multiple consecutive nucleotides.

In non-limiting embodiments of the invention, the presence of an allelic variation, for example a SNP, may be determined using a technique such as, but not limited to, primer extension or polymerase chain reaction, using primer(s) designed based on sequence in the proximity of the variation, followed by sequencing. For example, and not by way of limitation, the presence of a SNP may be determined by a method comprising using at least one primer sequence complementary to a sequence flanking the location of the SNP (for example, within 80 nucleotides, or within 50 nucleotides, or within 30 nucleotides, or within 20 nucleotides, or within 10 nucleotides, of the SNP) in a primer extension reaction or polymerase chain reaction to generate a test fragment that contains the location of the SNP and determining the nucleotide present at the location of the single nucleotide polymorphism, for example by sequencing all or a portion of the test fragment.

In addition to sequencing-based methods, other methods are known in the art for detecting one or more SNP including, but not limited to, methods that utilize hybridization, restriction fragment length polymorphism, enzyme-based methods, or electrophoresis, to name a few. Exemplary technologies for detecting SNPS include but are not limited to TaqMan® SNP Genotyping Assays, SNPlex™, Affymetrix Human SNP GeneChip (e.g. version 6.0), Sequenom MassArray sequencing, Illumina BeadArray products, and see, for example, De La Vega et al., 2005, Mutation Research/Fundamental and Molecular Mechanisms of Mutagenesis 573:111-135; Larmy et al. 2006, Nucl. Acids Res. 34(14):e100; Shen et al., 2005, Mutat. Res. 573(1-2):70-82; Gaudet et al., 2009, Methods Mol. Biol. 578:415-424; Shen et al., 2009, Methods Mol. Biol. 578:293-306; Olivier, 2005, Mutat. Res. 573(1-2):103-110; Duan et al., 2009, Biosens. Bioelectron 24(7): 2095-2099; Duan et al., 2009, Nat. Protoc. 4(6):984-991; Hori et al., 2003, Curr. Pharm. Biotechnol. 4(6):477-484; Kwok and Chen, 2003, Curr. Issues Mol. Biol. 5:43-60; Kwok, 2002, Hum. Mutat. 19(4):315-323 and Comen et al., 2011, Breast Cancer Res. Treat. 127(2):479-87.

Nucleic acid sequences of certain SNPs and the surrounding nucleic acids are set forth in TABLE 7 below.

In non-limiting embodiments where a biomarker is a protein, methods standard to the art may be used to detect the protein biomarker. Such methods include, but are not limited to, electrophoretic or chromatographic methods, mass spectrometry techniques, peptide sequencing, 1-D or 2-D gel-based analysis systems, protein microarray, immunofluorescence, Western blotting, enzyme linked immunosorbent assay (“ELISA”), radioimmunoassay (RIA), enzyme immunoassays (EIA) and other antibody-mediated detection methods known in the art.

A “subject” herein is a human subject. In particular non-limiting embodiments, the subject is a female. In particular non-limiting embodiments, the subject has a family history of breast cancer, for example, a mother, sister, grandmother, or aunt with breast cancer, for example occurring before the age of 40 or before the age of 50.

### 5.1 the BRCA2 Modifier Locus and its Biomarkers

In various embodiments a SNP (variant, minor allele) in the 6p24 region (also referred to herein as a “6p24 SNP”) of the human genome may be used to assess the risk that a subject also carrying a BRCA2 mutation (variant, minor allele) may have or develop breast cancer, where the presence of the 6p24 SNP is associated with a lower risk that the subject has or will develop breast cancer relative to a subject having a BRCA2 mutation (also referred to as a BRCA2 biomarker) and the major allele at the 6p24 position. In certain non-limiting embodiments, the location of the SNP is between human chromosome 6 position 10540 kb and 10570 kb or between 10550 kb and 10565 kb or between 10560 kb and 10565 kb. In certain non-limiting embodiments, r² for the association between the SNP and breast cancer is at least 0.6 or at least 0.7 or at least 0.8.

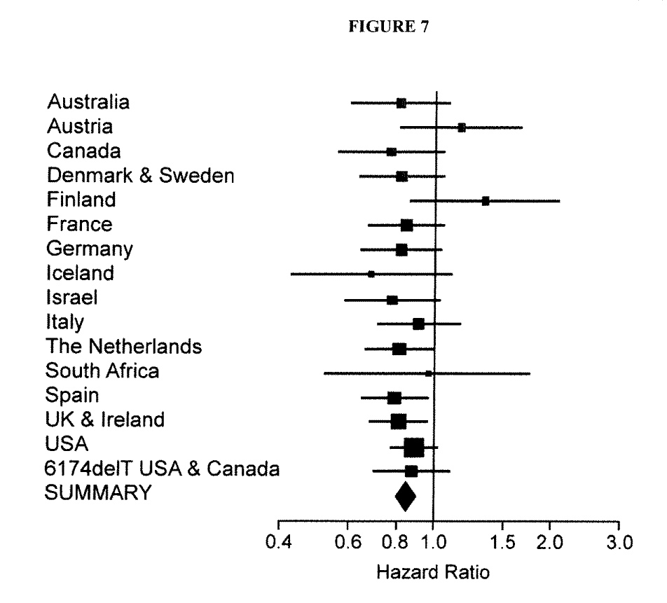

In a specific non-limiting embodiment, the SNP is the minor allele at rs9348512, which, as illustrated in the working example below, was observed to be associated with a 15% decreased risk of breast cancer among BRCA2 mutation carriers (per allele HR=0.85, 95% CI 0.80-0.90) with no evidence of between-country heterogeneity (P=0.78, FIG. 7). The association with rs9348512 did not differ by 6174delT mutation status (P for difference=0.33), age (P=0.39), or estrogen receptor (ER) status of the breast tumor (P=0.78).

In a particular non-limiting embodiment, the presence of the major allele in a subject may be evaluated, for example as a means of determining heterozygosity or as a cross check when assessing whether the minor allele is present.

In other specific non-limiting embodiments, the SNP in the 6p24 region is a SNP listed in TABLE 5 below, where the minor alleles are shown. For example, but not by way of limitation, additional examples of 6p24 SNPs, which may be used according to the invention, include (minor alleles of) rs9358529, rs303067 and rs9366443.

### 5.2 Risk Assessment Biomarker Panels

A 6p24 SNP may be used alone or together with one or more additional biomarker (which together constitute a “panel”) to assess the risk that a subject has or will develop breast cancer.

As the association for the 6p24 SNP with breast cancer is in the context of a BRCA2 mutation, a BRCA2 mutation, for example, but not limited to a BRCA2 SNP, may optionally be included in the panel so that the existence of BRCA2 mutation and 6p24 SNP may be assessed in the same assay or series of assays.

In addition, the panel may further comprise one or more biomarker in one or more or two or more or three or more or four or more or five or more or six or more or seven or more or eight or more or nine or more or ten or more or eleven or more or twelve or thirteen of the following genes and/or loci (“auxiliary biomarkers” in that they are other than a 6p24 SNP or a BRCA2 biomarker): 10q26 (FGFR2), 16q12 (TOX3), 12p11 (PTHLH), 5q11 (MA P3K1), 9p21 (CDKN2A/B), 11p15 (LSP1), 8q24, 6q25 (ESR1), 10q21 (ZNF365), 3p24 (SLC4A7, NEK10), 12q24, 5p12, and 11 q13. A non-limiting set of SNPs corresponding to these biomarkers is provided in TABLES 1, 2, 5, and 6. TABLE 1 provides information regarding which biomarkers are associated with an increased risk of breast cancer and which biomarkers are associated with a decreased risk of breast cancer.

In certain non-limiting embodiments, a panel may comprise, in addition to a 6p24 SNP and optionally BRCA2, one or more biomarker selected from the biomarkers set forth in TABLES 1, 2, and 6.

In certain non-limiting embodiments, the panel may comprise, in addition to a 6p24 SNP and optionally BRCA2, one or both of the following biomarkers: 10q26 (FGFR2) and/or 16q12 (TOX3).

In certain non-limiting embodiments, the panel may comprise, in addition to a 6p24 SNP and optionally BRCA2, one, two, three, four, five or six of the following biomarkers: 10q26 (FGFR2), 16q12 (TOX3), 5q11 (MAP3K1), 11p15 (LSP1) and/or 3p24 (SLC4A7, NEK10).

In a specific non-limiting embodiment, a panel comprises a biomarker of 10q26 (FGFR2) which is the SNP rs2420946.

In a specific, non-limiting embodiment, a panel comprises a biomarker of 16q12 (TOX3) which is the SNP rs3803662.

In a specific non-limiting embodiment, a panel comprises a biomarker of 12p11 (PTHLH) which is the SNP rs27633.

In a specific non-limiting embodiment, a panel comprises a biomarker of 5q11 (MAP3K1) which is the SNP rs16886113.

In a specific non-limiting embodiment, a panel comprises a biomarker of 10q26 (CDKN2A/B) which is the SNP rs10965163.

In a specific non-limiting embodiment, a panel comprises a biomarker of 8q24 which is the SNP rs4733664.

In a specific non-limiting embodiment, a panel comprises a biomarker of 6q25 (ESR1) which is the SNP rs2253407.

In a specific non-limiting embodiment, a panel comprises a biomarker of 10q21 (ZNF365) which is the SNP rs17221319.

In non-limiting embodiments, the above-described panel of biomarkers may constitute at least 10% or at least 20% or at least 30% or at least 40% or at least 50% or at least 60% or at least 70% or at least 80% or at least 90% or at least 95% or 100% of the biomarkers tested in a panel.

Biomarkers, which are not SNPs, may be detected using methods known in the art. Materials for such methods are discussed in the kits section below.

### 5.3 Kits

In non-limiting embodiments, the present invention provides for a kit for determining whether a subject has an increased risk of having or developing breast cancer comprising a means for detecting a 6p24 SNP and one or more biomarker selected from BRCA2, and an auxiliary biomarker selected from the group of 10q26 (FGFR2), 16q12 (TOX3), 12p11 (PTHLH), 5q11 (MAP3K1), 9p21 (CDKN2A/B), 11p15 (LSP1), 8q24, 6q25 (ESR1), 10q21 (ZNF365), 3p24 (SLC4A7, NEK10), 12q24, 5p12, and 11q13.

Types of kits include, but are not limited to, packaged probe and primer sets (e.g. TaqMan probe/primer sets), arrays/microarrays, biomarker-specific antibodies and beads, which further contain one or more probes, primers, or other detection reagents for detecting one or more biomarkers of the present invention.

In a specific, non-limiting embodiment, a kit may comprise a pair of oligonucleotide primers, suitable for polymerase chain reaction (PCR) or nucleic acid sequencing, for detecting the biomarker(s) to be identified. A pair of primers may comprise nucleotide sequences complementary to a biomarker set forth above, and be of sufficient length to selectively hybridize with said biomarker. Alternatively, the complementary nucleotides may selectively hybridize to a specific region in close enough proximity 5′ and/or 3′ to the biomarker position to perform PCR and/or sequencing. Multiple biomarker-specific primers may be included in the kit to simultaneously assay large number of biomarkers. The kit may also comprise one or more polymerases, reverse transcriptase, and nucleotide bases, wherein the nucleotide bases can be further detectably labeled.

In non-limiting embodiments, a primer may be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In a specific, non-limiting embodiment, a kit may comprise at least one nucleic acid probe, suitable for in situ hybridization or fluorescent in situ hybridization, for detecting the biomarker(s) to be identified. Such kits will generally comprise one or more oligonucleotide probes that have specificity for various biomarkers.

In a further non-limiting embodiment, the oligonucleotide primers and/or probes may be immobilized on a solid surface or support, for example, on a nucleic acid microarray, wherein the position of each oligonucleotide primer and/or probe bound to the solid surface or support is known and identifiable.

In other non-limiting embodiments, a kit may comprise a primer for detection of a biomarker by primer extension.

In other non-limiting embodiments, a kit may comprise at least one antibody for immunodetection of the biomarker(s) to be identified. Antibodies, both polyclonal and monoclonal, specific for a biomarker, may be prepared using conventional immunization techniques, as will be generally known to those of skill in the art. The immunodetection reagents of the kit may include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (³H, ³⁵S, ³²P, ¹⁴C, ¹³¹I) or enzymes (alkaline phosphatase, horseradish peroxidase). Alternatively, a detectable moiety may be comprised in a secondary antibody or antibody fragment which selectively binds to the first antibody or antibody fragment (where said first antibody or antibody fragment specifically recognizes a biomarker).

In a further non-limiting embodiment, the biomarker-specific antibody may be provided bound to a solid support, such as a column matrix, an array, or well of a microtiter plate. Alternatively, the support may be provided as a separate element of the kit.

In one specific non-limiting embodiment, a kit may comprise a primer, a pair of primers, a probe, microarray, or antibody suitable for detecting a 6p24 SNP biomarker.

In one specific non-limiting embodiment, a kit may comprise a primer, a pair of primers, a probe, microarray, or antibody suitable for detecting the rs9348512 SNP.

In certain non-limiting embodiments, a kit may comprise one or more pair of primers, primer, probe, microarray, or antibody suitable for detecting, in addition to a 6p24 SNP and optionally BRCA2, one, two, three, four, five or six of the following biomarkers: 10q26 (FGFR2), 16q12 (TOX3), 5q11 (MAP3K1), 11p15 (LSP1) and/or 3p24 (SLC4A7, NEK10).

In certain non-limiting embodiments, a kit may comprise a primer, a pair of primers, a probe, microarray, or antibody suitable for detecting, in addition to a 6p24 SNP and optionally BRCA2, one or more of the biomarkers shown in TABLES 1, 2 and 6.

In a specific non-limiting embodiment, a kit may comprise a primer, a pair of primers, a probe, microarray, or antibody suitable for detecting a biomarker of 10q26 (FGFR2) which is the SNP rs2420946.

In a specific, non-limiting embodiment, a kit may comprise a primer, a pair of primers, a probe, microarray, or antibody suitable for detecting a biomarker of 16q12 (TOX3) which is the SNP rs3803662.

In a specific non-limiting embodiment, a kit may comprise a primer, a pair of primers, a probe, microarray, or antibody suitable for detecting a biomarker of 12p11 (PTHLH) which is the SNP rs27633.

In a specific non-limiting embodiment, a kit may comprise a primer, a pair of primers, a probe, microarray, or antibody suitable for detecting a biomarker of 5q11 (MAP3K1) which is the SNP rs16886113.

In a specific non-limiting embodiment, a kit may comprise a primer, a pair of primers, a probe, microarray, or antibody suitable for detecting a biomarker of 10q26 (CDKN2A/B) which is the SNP rs10965163.

In a specific non-limiting embodiment, a kit may comprise a primer, a pair of primers, a probe, microarray, or antibody suitable for detecting a biomarker of 8q24 which is the SNP rs4733664.

In a specific non-limiting embodiment, a kit may comprise a primer, a pair of primers, a probe, microarray, or antibody suitable for detecting a biomarker of 6q25 (ESR1) which is the SNP rs2253407.

In a specific non-limiting embodiment, a kit may comprise a primer, a pair of primers, a probe, microarray, or antibody suitable for detecting a biomarker of 10q21 (ZNF365) which is the SNP rs17221319.

In certain non-limiting embodiments, where the measurement means in the kit employs an array, the set of biomarkers set forth above may constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a biomarker detection kit may comprise one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a biomarker.

A kit may further contain means for comparing the biomarker with a standard, and can include instructions for using the kit to detect the biomarker of interest. Specifically, the instructions describes that the presence of a biomarker, set forth herein, is indicative of an increased or decreased risk that a subject has or will develop breast cancer.

### 5.4 Prognostic Methods

In non-limiting embodiments, the present invention provides for a method for assessing the likelihood that a subject has or will develop breast cancer comprising determining whether the subject carries a 6p24 SNP biomarker and a BRCA2 biomarker, where the presence of both biomarkers indicates that while the subject has an increased risk of having or developing breast cancer relative to the general population, the risk is less than if the 6p24 biomarker were absent. In a non-limiting subset of embodiments, it is already known that the subject carries a BRCA2 biomarker (mutation, minor allele) so that the method need not necessarily re-test for the presence of that biomarker, although it may be desirable as confirmation or supporting information.

In non-limiting embodiments of said method, the 6p24 SNP biomarker is rs9348512 SNP.

In non-limiting embodiments, said method may further comprise determining whether the subject carries one or more auxiliary biomarker selected from the group of 10q26 (FGFR2), 16q12 (TOX3), 12p11 (PTHLH), 5q11 (MAP3K1), 9p21 (CDKN2A/B), 11p15 (LSP1), 8q24, 6q25 (ESR1), 10q21 (ZNF365), 3p24 (SLC4A7, NEK10), 12q24, 5p12, and 11q13. The effect of the presence or absence of said biomarker(s) on the risk of having or developing breast cancer is presented in TABLE 1.

In certain non-limiting embodiments, said method may further comprise determining whether the subject carries one or more biomarker selected from the biomarkers shown in TABLES 1, 2, and 6. The effect of the presence or absence of said biomarker(s) on the risk of having or developing breast cancer is presented in TABLES 1, 2, and/or 6.

The presence of a biomarker may be determined in the subject in vivo or in a sample collected from the subject using methods known in the art. Non-limiting examples of a sample include, but are not limited to, a clinical sample, a tumor sample, cells in culture, cell supernatants, lymphocytes, an exudate, cell lysates, serum, blood plasma, biological fluid (e.g., lymphatic fluid), and tissue samples. The source of the sample may be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid, saliva, or interstitial fluid), or cells from the individual, including circulating tumor cells.

Methods for determining the presence of biomarker are known in the art and are discussed above.

The foregoing method may comprise the further step, where the subject is found to carry a 6p24 SNP, of recommending or performing regular breast screening to monitor for the presence of cancer, for example by clinical breast exam, breast biopsy, mammography, ultrasound, magnetic resonance imaging, or similar techniques. Regular screening may, in non-limiting embodiments, be at least four times a year, at least twice a year, at least once a year, or every two years.

### 5.5 Methods of Treatment

In certain non-limiting embodiments, the present invention provides for a method of treating a subject who carries a BRCA2 biomarker (mutation), comprising determining whether the subject carries a 6p24 SNP biomarker and, where the 6p24 SNP biomarker is absent, advising the subject that she is at high risk for developing breast cancer relative to a subject carrying both the 6p24 SNP and BRCA2 biomarkers and to the general population. In a subset of non-limiting embodiments, the subject is previously known to carry the BR CA 2 biomarker (mutation); alternatively, both the 6p24 SNP and BRCA2 biomarkers may be assessed.

In non-limiting embodiments of said method, the 6p24 SNP biomarker is rs9348512 SNP.

In certain non-limiting embodiments, the method may further comprise determining whether the subject carries one or more auxiliary biomarker selected from the group of 10q26 (FGFR2), 16q12 (TOX3), 12p11 (PTHLH), 5q11 (MAP3K1), 9p21 (CDKN2A/B), 11p15 (LSP1), 8q24, 6q25 (ESR1), 10q21 (ZNF365), 3p24 (SLC4A7, NEK10), 12q24, 5p12 and 11q13. The effect of the presence or absence of said biomarker(s) on the risk of having or developing breast cancer is presented in TABLE 1.

In certain non-limiting embodiments, the method may further comprise determining whether the subject carries one or more biomarker selected from the biomarkers shown in TABLES 1, 2, and 6. The effect of the presence or absence of said biomarker(s) on the risk of having or developing breast cancer is presented in TABLES 1, 2, and/or 6.

The presence of a biomarker may be determined in the subject in vivo or in a sample collected from the subject using methods known in the art. Non-limiting examples of a sample include, but are not limited to, a clinical sample, a tumor sample, cells in culture, cell supernatants, lymphocytes, an exudate, cell lysates, serum, blood plasma, biological fluid (e.g., lymphatic fluid), and tissue samples. The source of the sample may be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid, saliva, or interstitial fluid), or cells from the individual, including circulating tumor cells.

Methods for determining the presence of biomarker are known in the art and are discussed above.

The foregoing method may comprise the further step, where the subject is found not to carry the 6p24 SNP, of recommending or performing a mastectomy or oophorectomy, or initiating anti-estrogen therapy or chemoprevention. In addition the biomarker profile may serve to guide targeted therapies against tyrosine kinase pathways which are implicated by many of the biomarkers included in the panel, and which may down-modulate risk in BRCA2 mutation carriers. The protein products of the biomarkers themselves may serve as therapeutic targets to down-modulate breast cancer risk.

### 6. EXAMPLE

### Identification of a BRCA2-Specific Modifier Locus at 6p24 Related to Breast Cancer Risk

### 6.1 Materials and Methods

Ethics Statement.

Each of the host institutions (TABLE 4) recruited under ethically-approved protocols. Written informed consent was obtained from all subjects.

Study Subjects.

The majority of BRCA2 mutation carriers were recruited through cancer genetics clinics and some came from population or community-based studies. Studies contributing DNA samples to these research efforts were members of the Consortium of Investigators of Modifiers of BRCA1/2 (CIMBA) with the exception of one study (NICCC). Eligible subjects were women of European descent who carried a pathogenic BRCA2 mutation, had complete phenotype information, and were at least 18 years of age. Harmonized phenotypic data included year of birth, age at cancer diagnosis, age at bilateral prophylactic mastectomy and oophorectomy, age at interview or last follow-up, BRCA2 mutation description, self-reported ethnicity, and breast cancer estrogen receptor status.

GWAS Discovery Stage Samples.

Details of these samples have been described previously [2]. Data from 899 young (<40 years) affected and 804 older (>40 years) unaffected carriers of European ancestry from 14 countries were used to select SNPs for inclusion on the iCOGS array.

Samples Genotyped in the Extended Replication Set.

Forty-seven studies from 24 different countries (including two East-Asian countries) provided DNA from a total of 10,048 BRCA2 mutations carriers. All eligible samples were genotyped using COGs, including those from the discovery stage.

Genotyping and Quality Control.
BRCA2 SNP Selection for Inclusion on iCOGS.

The Collaborative Oncological Gene-Environment Study (COGS) consortium developed a custom genotyping array (referred to as the iCOGS array) to provide efficient genotyping of common and rare genetic variants to identify novel loci that are associated with risk of breast, ovarian, and prostate cancers as well as to fine-map known cancer susceptibility loci. SNPs were selected for inclusion on iCOGS separately by each participating consortium: Breast Cancer Association Consortium (BCAC) [6], Ovarian Cancer Association Consortium (OCAC) [7], Prostate Cancer Association Group to Investigate Cancer Associated Alterations in the Genome (PRACTICAL) [8], and CIMBA. SNP lists from a BRCA1 GWAS and SNPs in candidate regions were used together with the BRCA2 GWAS lists to generate a ranked CIMBA SNP list that included SNPs with the following nominal proportions: 55.5% from the BRCA1 GWAS, 41.6% from the BRCA2 GWAS and fine mapping, 2.9% for CIMBA candidate SNPs. Each consortium was given a share of the array: nominally 25% of the SNPs each for BCAC, PRACTICAL and OCAC; 17.5% for CIMBA; and 7.5% for SNPs from commonly researched pathways (e.g., inflammation). For the CIMBA BRCA2 GWAS, we used the iCOGS array as the platform to genotype the extended replication set of the discovery GWAS stage [2]. SNPs were selected on the basis of the strength of their associations with breast cancer risk in the discovery stage [2], using imputed genotype data for 1.4M SNPs identified through CEU+TSI samples on HapMap3, release 2. A ranked list of SNPs was based on the 1-df trend test statistic, after excluding highly correlated SNPs (r2>0.4). The final list included the 39,015 SNPs with the smallest p-values. An additional set of SNPs were selected for fine mapping of the regions surrounding the SNPs found to be associated with breast cancer in the discovery GWAS stage: rs16917302 on 10q21 and rs311499 on 20q13, including SNPs with a MAF >0.05 located 500 kb in both directions of the SNP, based on HapMap 2 data. The final combined list of SNPs for the iCOGS array comprised 220,123 SNPs. Of these, 211,155 were successfully manufactured onto the array. The present analyses are based on the 19,029 SNPs selected on the basis of BRCA2 GWAS and fine mapping that were included on the iCOGS array.
Genotyping.

The genotyping was performed on DNA samples from 10,048 BRCA2 mutation carriers at the McGill University and Genome Québec Innovation Centre (Montreal, Canada). As a quality control measure, each plate included DNA samples from six individuals who were members of two CEPH trios. Some plates also contained three duplicate pairs of quality control samples. Genotypes were called using GenCall [9]. Initial calling was based on a cluster file generated using 270 samples from Hapmap2. To generate the final calls, we first selected a subset of 3,018 individuals, including samples from each of the genotyping centers in the iCOGS project, each of the participating consortia, and each major ethnicity. Only plates with a consistent high call rate in the initial calling were used. We also included 380 samples of European, African, and Asian ethnicity genotyped as part of the Hapmap and 1000 Genomes project, and 160 samples that were known positive controls for rare variants on the array. This subset was used to generate a cluster file that was then applied to call the genotypes for the remaining samples.

Quality Control of SNPs.

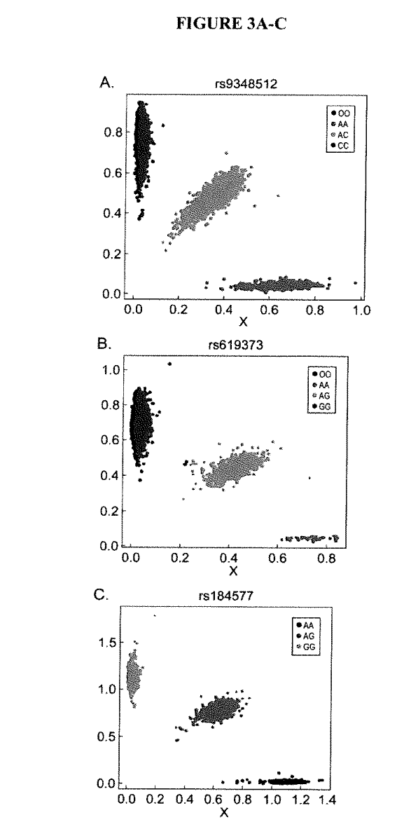

Of the 211,155 SNPs on the iCOGS array, we excluded SNPs for the following reasons (TABLE 4): on the Y-chromosome, call rate <95%, deviations from Hardy-Weinberg equilibrium (P<10-7) using a stratified 1-d.f. test [10], and monomorphic. SNPs that gave discrepant genotypes among known duplicates were also excluded. After quality control filtering, 200,908 SNPs were available for analysis (TABLE 3); 18,086 of which were selected on the basis of the discovery BRCA2 GWAS [2]. Cluster plots of all reported SNPs were inspected manually for quality (FIG. 3).

Description of Imputation.

Genotypes for SNPs identified through the 1000 Genomes Phase I data (released January 2012) [11] were imputed using all SNPs on the iCOGS chip in a region of 500 kb around the novel modifier locus at 6p24. The boundaries were determined according to the linkage disequilibrium (LD) structure in the region based on HapMap data. The imputation was carried out using IMPUTE 2.2 [12]. SNPs with imputation information/accuracy r²<0.30 were excluded in the analyses.

Quality Control of DNA Samples.

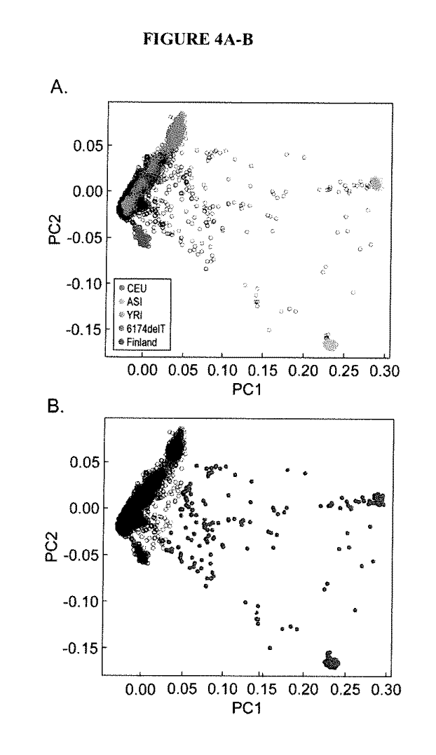

Of 10,048 genotyped samples (TABLE 3), 742 were excluded because they did not meet the phenotypic eligibility criteria or had self-reported non-CEU ethnicity (TABLE 4). Samples were then excluded for the following reasons: not female (XXY, XY), call rate <95%, low or high heterozygosity (P<10-6), discordant genotypes from previous CIMBA genotyping efforts, or discordant duplicate samples. For duplicates with concordant phenotypic data, or in cases of cryptic monozygotic twins, only one of the samples was included. Cryptic duplicates for which phenotypic data indicated different individuals were all excluded. Samples of non-European ancestry were identified using multi-dimensional scaling, after combining the BRCA2 mutation carrier samples with the HapMap2 CEU, CHB, JPT and YRI samples using a set of 37,120 uncorrelated SNPs from the iCOGS array. Samples with >19% non-European ancestry were excluded (FIG. 4). A total of 4,330 affected and 3,881 unaffected BRCA2 mutation carrier women of European ancestry from 42 studies remained in the analysis (TABLE 4), including 3,234 breast cancer cases and 3,490 unaffected carriers that were not in the discovery set.

BRCA1 and BCAC Samples.

Details of the sample collection, genotyping and quality control process for the BRCA1 and BCAC samples, are reported elsewhere [13,14].

Statistical Methods.

The associations between genotype and breast cancer risk were analyzed within a retrospective cohort framework with time to breast cancer diagnosis as the outcome [15]. Each BRCA2 carrier was followed until the first of: breast or ovarian cancer diagnosis, bilateral prophylactic mastectomy, or age at last observation. Only those with a breast cancer diagnosis were considered as cases in the analysis. The majority of mutation carriers were recruited through genetic counseling centers where genetic testing is targeted at women diagnosed with breast or ovarian cancer and in particular to those diagnosed with breast cancer at a young age. Therefore, these women are more likely to be sampled compared to unaffected mutation carriers or carriers diagnosed with the disease at older ages. As a consequence, sampling was not random with respect to disease phenotype and standard methods of survival analysis (such as Cox regression) may lead to biased estimates of the associations [16]. We therefore conducted the analysis by modelling the retrospective likelihood of the observed genotypes conditional on the disease phenotypes. This has been shown to provide unbiased estimates of the associations [15]. The implementation of the retrospective likelihoods has been described in detail elsewhere [15,17]. The associations between genotype and breast cancer risk were assessed using the 1 degree of freedom score test statistic based on the retrospective likelihood [15]. In order to account for non-independence between relatives, an adjusted version of the score test was used in which the variance of the score was derived taking into account the correlation between the genotypes [18]. P-values were not adjusted using genomic control because there was little evidence of inflation. Inflation was assessed using the genomic inflation factor λ. Since this estimate is dependent on sample size, we also calculated λ adjusted to 1000 affected and 1000 unaffected samples. Per-allele and genotype-specific hazard-ratios (HR) and 95% confidence intervals (CI) were estimated by maximizing the retrospective likelihood. Calendar-year and cohort-specific breast cancer incidences for BRCA2 were used [1]. All analyses were stratified by country of residence. The USA and Canada strata were further subdivided by reported Ashkenazi Jewish ancestry. The assumption of proportional hazards was assessed by fitting a model that includes a genotype-by-age interaction term. Between-country heterogeneity was assessed by comparing the results of the main analysis to a model with country-specific log-HRs. A possible survival bias due to inclusion of prevalent cases was evaluated by re-fitting the model after excluding affected carriers that were diagnosed ≧5 years prior to study recruitment. The associations between genotypes and tumor subtypes were evaluated using an extension of the retrospective likelihood approach that models the association with two or more subtypes simultaneously [19]. To investigate whether any of the significant SNPs were associated with ovarian cancer risk for BRCA2 mutation carriers and whether the inclusion of ovarian cancer patients as unaffected subjects biased our results, we also analyzed the data within a competing risks framework and estimated HR simultaneously for breast and ovarian cancer using the methods described elsewhere [15]. Analyses were carried out in R using the GenABEL libraries [20] and custom-written software. The retrospective likelihood was modeled in the pedigree-analysis software MENDEL [21], as described in detail elsewhere [15].

TCGA Analysis. Affymetrix SNP 6.0 genotype calls for normal (non-tumor) breast DNA were downloaded for all available individuals from The Cancer Genome Atlas in September 2011. Analyses were limited to the 401 individuals of European ancestry based on principal component analysis. Expression levels in breast tumor tissue were adjusted for the top two principal components, age, gender (there are some male breast cancer cases in TCGA), and average copy number across the gene in the tumor. Linear regression was then used to test for association between the SNP and the adjusted gene expression level for all genes within one megabase.

Gene Set Enrichment Analysis.

To investigate enrichment of genes associated with breast cancer risk, the gene-set enrichment approach was implemented using Versatile Gene-based Association Study [22] based on the ranked P-values from retrospective likelihood analysis. Association List Go Annotator was also used to prioritize gene pathways using functional annotation from gene ontology (GO) [23] to increase the power to detect association to a pathway, as opposed to individual genes in the pathway. Both analyses were corrected for LD between SNPs, variable gene size, and interdependence of GO categories, where applicable, based on imputation. 100,000 Monte Carlo simulations were performed in VEGAS and 5000 replicate gene lists using random sampling of SNPs and 5000 replicate studies (sampling with replacement) were performed to estimate P-values.

Predicted Absolute Breast Cancer Risks by Combined SNP Profile.

We estimated the absolute risks of developing breast cancer based on the joint distribution of SNPs associated with breast cancer for BRCA2 mutation carriers. The methods have been described elsewhere [24]. To construct the SNP profiles, we considered the single SNP from each region with the strongest evidence of association in the present dataset. We included all loci that had previously been found to be associated with breast cancer risk through GWAS in the general population and demonstrated associations with breast cancer risk for BRCA2 mutation carriers, and loci that had GWAS level of significance in the current study. We assumed that all loci in the profile were independent (i.e., they interact multiplicatively on BRCA2 breast cancer risk). Genotype frequencies were obtained under the assumption of Hardy-Weinberg Equilibrium. For each SNP, the effect of each allele was assumed to be consistent with a multiplicative model (log-additive). We assumed that the average, age-specific breast cancer incidences, over all associated loci, agreed with published breast cancer risk estimates for BRCA2 mutation carriers [1].

### 6.2 Results

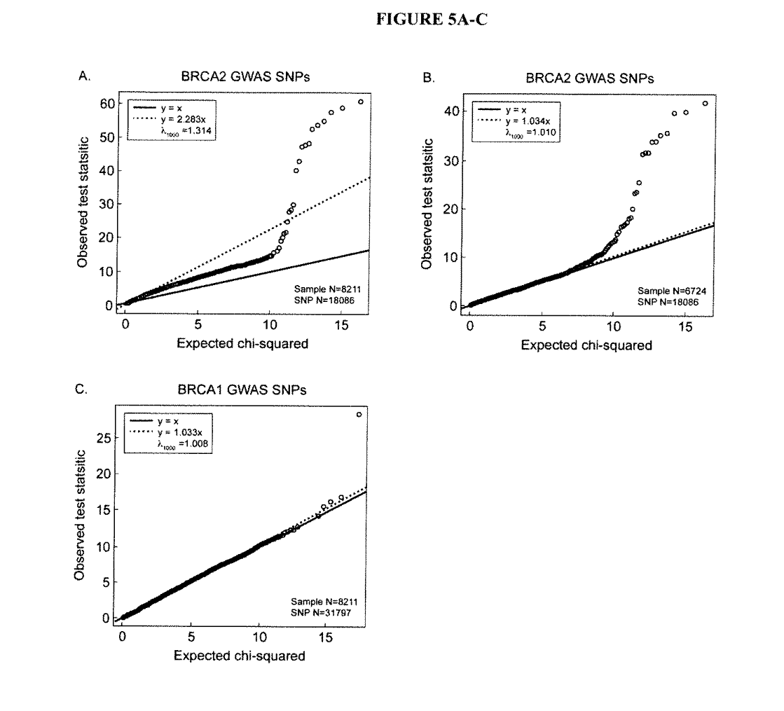

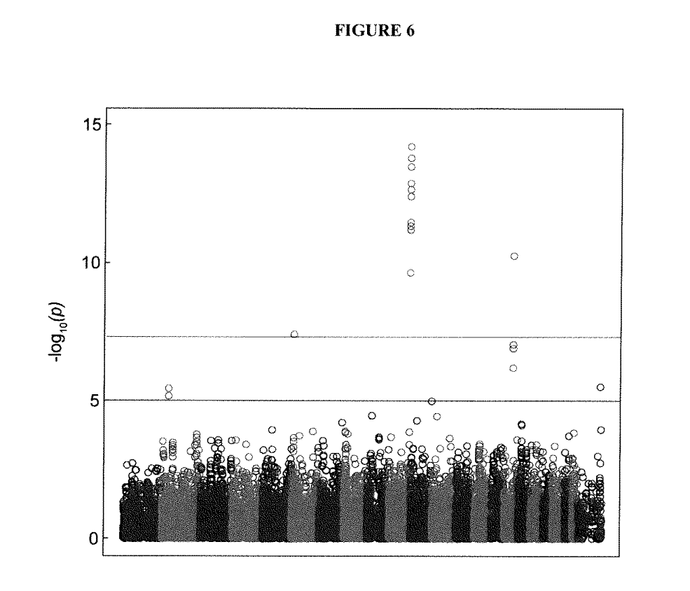

The genomic inflation factor (λ) based on the 18,086 BRCA2 GWAS SNPs in the 6,724 BRCA2 mutation carriers not used for SNP discovery was 1.034 (λ adjusted to 1000 affected and 1000 unaffected: 1.010, FIG. 5). Multiple variants were associated with breast cancer risk in the combined discovery and replication datasets (FIG. 6). SNPs in three independent regions had P-values <5×10⁻⁸; one was a region not previously associated with breast cancer.

The most significant associations were observed for known breast cancer susceptibility regions, rs2420946 (per allele P=2×10⁻¹⁴) in FGFR2 and rs3803662 (P=5.4×10⁻¹¹) near TOX3 (TABLE 1). Breast cancer risk associations with other SNPs reported previously for BRCA2 mutation carriers are summarized in TABLE 1. In this larger set of BRCA2 mutation carriers, we also identified novel SNPs in the 12p11 (PTHLH), 5q11 (MAP3K1), and 9p21 (CDKN2A/B) regions with smaller P-values for association than those of previously reported SNPs. These novel SNPs were not correlated with the previously reported SNPs (r²<0.14). For one of the novel SNPs identified in the discovery GWAS [2], ZNF365 rs16917302, there was weak evidence of association with breast cancer risk (P=0.01); however, an uncorrelated SNP, rs17221319 (r²<0.01), 54 kb upstream of rs16917302, had stronger evidence of association (P=6×10-3).

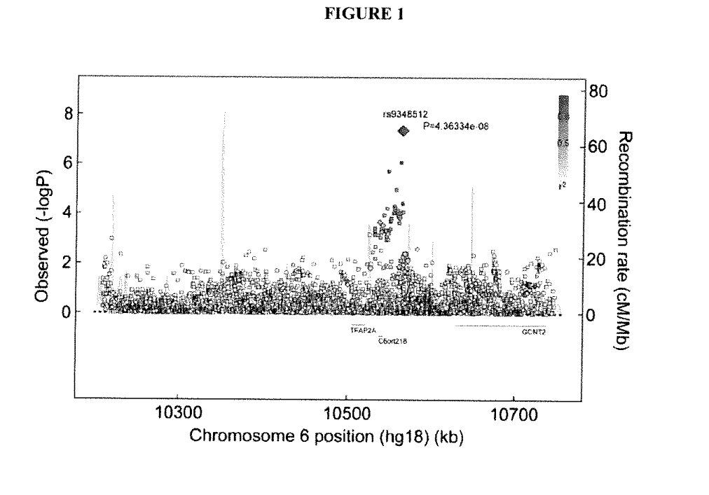

One SNP, rs9348512 at 6p24, not known to be associated with breast cancer, had a combined P-value of association of 3.9×10⁻⁸ amongst all BRCA2 samples (TABLE 2), with strong evidence of replication in the set of BRCA2 samples that were not used in the discovery stage (P=5.2×10⁻⁵). The minor allele of rs9348512 (MAF-0.35) was associated with a 15% decreased risk of breast cancer among BRCA2 mutation carriers (per allele HR=0.85, 95% CI 0.80-0.90) with no evidence of between-country heterogeneity (P=0.78, FIG. 7). None of the genotyped (n=68) or imputed (n=3,507) SNPs in that region showed a stronger association with risk (FIG. 1; TABLE 5), but there were 40 SNPs with P<10⁻⁴ (pairwise r²>0.38 with rs9348512, with the exception of rs11526201 for which r²=0.01, TABLE 5). The association with rs9348512 did not differ by 6174delT mutation status (P for difference=0.33), age (P=0.39), or estrogen receptor (ER) status of the breast tumor (P=0.78). Exclusion of prevalent breast cancer cases (n=1,752) produced results (HR=0.83, 95% CI 0.77-0.89, P=3.40×10⁻⁷) consistent with those for all cases.

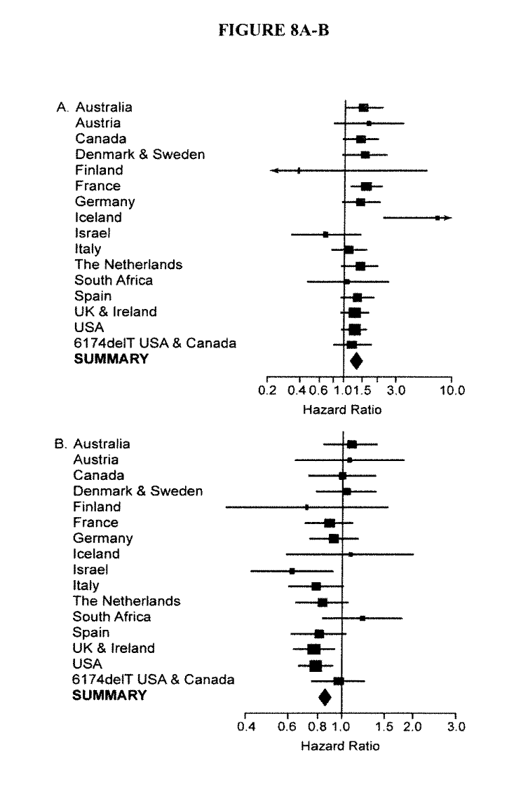

SNPs in two additional regions had P-values <10⁻⁵ for breast cancer risk associations for BRCA2 mutation carriers (TABLE 2). The magnitude of associations for both SNPs was similar in the discovery and second stage samples. In the combined analysis of all samples, the minor allele of rs619373, located in FGF13 (Xq26.3), was associated with higher breast cancer risk (HR=1.30, 95% CI 1.17-1.45, P=3.1×10-6). The minor allele of rs184577, located in CYP1B1-AS1 (2p22-p21), was associated with lower breast cancer risk (HR=0.85, 95% CI 0.79-0.91, P=3.6×10-6). These findings were consistent across countries (P for heterogeneity between country strata=0.39 and P=0.30, respectively; FIG. 8). There was no evidence that the HR estimates for rs619373 and rs184577 change with age of the BRCA2 mutation carriers (P for the genotype-age interaction=0.80 and P=0.40, respectively) and no evidence of survival bias for either SNP (rs619373: HR=1.35, 95% CI 1.20-1.53, P=1.5×10-6 and rs184577: HR=0.86, 95% CI 0.79-0.93, P=2.0×10-4, after excluding prevalent cases). The estimates for risk of ER-negative and ER-positive breast cancer were not significantly different (P for heterogeneity between tumor subtypes=0.79 and 0.67, respectively). When associations were evaluated under a competing risks model, there was no evidence of association with ovarian cancer risk for SNPs rs9348512 at 6p24, rs619373 in FGF13 or rs184577 at 2p22 and the breast cancer associations were virtually unchanged (TABLE 6).

Gene set enrichment analysis confirmed that strong associations exist for known breast cancer susceptibility loci and the novel loci identified here (gene-based P<1×10⁻5). The pathways most strongly associated with breast cancer risk that contained statistically significant SNPs included those related to ATP binding, organ morphogenesis, and several nucleotide bindings (pathway-based P<0.05).

To begin to determine the functional effect of rs9348512, we examined associations of expression levels of any nearby gene in breast tumors with the minor A allele. Using data from The Cancer Genome Atlas, we found that the A allele of rs9348512 was strongly associated with mRNA levels of GCNT2 in breast tumors (p=7.3×10⁻⁵).

### 6.3 Discussion

In the largest assemblage of BRCA2 mutation carriers, we identified a novel locus at 6q24 that is associated with breast cancer risk, and noted two potential SNPs of interest at Xq26 and 2p22. We also replicated associations with known breast cancer susceptibility SNPs previously reported in the general population and in BRCA2 mutation carriers. For the 12p11 (PTHLH), 5q11 (MAP3K1), and 9p21 (CDKN2A/B), we found uncorrelated SNPs that had stronger associations than the originally identified SNP in the breast cancer susceptibility region that should be replicated in the general population. In BRCA2 mutation carriers, evidence for a breast cancer association with genetic variants in PTHLH has been restricted previously to ER-negative tumors [25]; however, the novel susceptibility variant we reported here was associated with risk of ER+ and ER− breast cancer.

The novel SNP rs9348512 (6p24) is located in a region with no known genes (FIG. 1). C6orf218, a gene encoding a hypothetical protein LOC221718, and a possible tumor suppressor gene, TFAP2A, are within 100 kb of rs9348512. TFAP2A encodes the AP-2α transcription factor that is normally expressed in breast ductal epithelium nuclei, with progressive expression loss from normal, to ductal carcinoma in situ, to invasive cancer [26,27]. AP-2α also acts as a tumor suppressor via negative regulation of MYC [28] and augmented p53-dependent transcription [29]. However, the minor allele of rs9348512 was not associated with gene expression changes of TFAP2A in breast cancer tissues in The Cancer Genome Atlas (TCGA) data; this analysis might not be informative since expression of TFAP2A in invasive breast tissue is low [26,27]. Using the TCGA data and a 1 Mb window, expression changes with genotypes of rs9348512 were observed for GCNT2, the gene encoding the enzyme for the blood group 1 antigen glucosaminyl (N-acetyl) transferase 2. GCNT2, recently found to be overexpressed in highly metastatic breast cancer cell lines [30] and basal-like breast cancer [31], interacts with TGF-β to promote epithelial-to-mesenchymal transition, enhancing the metastatic potential of breast cancer [31]. An assessment of alterations in expression patterns in normal breast tissue from BRCA2 mutation carriers by genotype are needed to further evaluate the functional implications of rs9348512 in the breast tumorigenesis of BRCA2 mutation carriers.

To determine whether the breast cancer association with rs9348512 was limited to BRCA2 mutation carriers, we compared results to those in the general population genotyped by BCAC and to BRCA1 mutation carriers in CIMBA. No evidence of an association between rs9348512 and breast cancer risk was observed in the general population (OR=1.00, 95% CI 0.98-1.02, P=0.74) [14], nor in BRCA1 mutation carriers (HR=0.99, 95% CI 0.94-1.04, P=0.75) [13]. Stratifying cases by ER status, there was no association observed with ER-subtypes in either the general population or among BRCA1 mutation carriers (BCAC: ER positive P=0.89 and ER negative P=0.60; CIMBA BRCA1: P=0.49 and P=0.99, respectively). For the two SNPs associated with breast cancer with P<10-5, neither rs619373, located in FGF13 (Xq26.3), nor rs184577, located in CYP1B1-AS1 (2p22-p21), was associated with breast cancer risk in the general population [14] or among BRCA1 mutation carriers [13]. The narrow CIs for the overall associations in the general population and in BRCA1 mutation carriers rule out associations of magnitude similar to those observed for BRCA2 mutation carriers. The consistency of the association in the discovery and replication stages and by country, the strong quality control measures and filters, and the clear cluster plot for rs9348512 suggest that our results constitute the discovery of a novel breast cancer susceptibility locus specific to BRCA2 mutation carriers rather than a false positive finding.

rs9348512 (6p24) is the first example of a common susceptibility variant identified through GWAS that modifies breast cancer risk specifically in BRCA2 mutation carriers. Previously reported BRCA2-modifying alleles for breast cancer, including those in FGFR2, TOX3, MAP3K1, LSP1, 2q35, SLC4A7, 5p12, 1p11.2, ZNF365, and 19p13.1 (ER-negative only) [18,32,33], are also associated with breast cancer risk in the general population and/or BRCA1 mutation carriers.

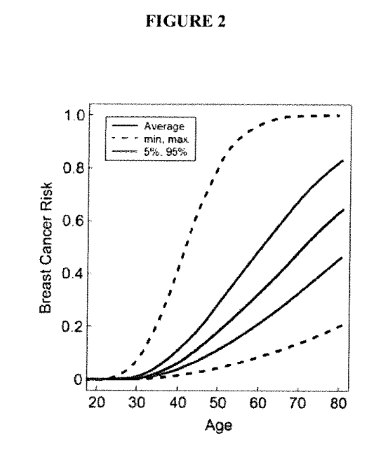

Taking into account all loci associated with breast cancer risk in BRCA2 mutation carriers from the current analysis, including the 6p24 locus, the 5% of the BRCA2 mutation carriers at lowest risk were predicted to have breast cancer risks by age 80 in the range of 21-47% compared to 83-100% for the 5% of mutation carriers at highest risk on the basis of the combined SNP profile distribution (FIG. 2). The breast cancer risk by age 50 is predicted to be 4-11% for the 5% of the carriers at lowest risk compared to 29-81% for the 5% at highest risk.

TABLE 1
Per allele hazard ratios (HR) and 95% confidence intervals (CI) of previously published breast cancer loci among
BRCA2 mutation carriers from previous reports and from the iCOGS array, ordered by statistical significance of the region

| | | | | | Previously Reported Results | | | | |
|---|---|---|---|---|---|---|---|---|---|
| Chr | Report | | | Minor | | Affected | Unaffected | Per Allele | p- |
| (Nearby Genes) | Status¹ | SNP | r² | Allele | Ref | N | N | HR (95% CI) | value² |
| 10q26 | reported | rs2981575 | 0.96 | G | [2] | 2,155 | 2,016 | 1.28 (1.18, 1.39) | 1 × 10⁻⁸ |
| (FGFR2) | novel | rs2420946 | | A | | | | | |
| 16q12 | reported | rs3803662 | | A | [2] | 2,162 | 2,026 | 1.20 (1.10, 1.31) | 5 × 10⁻⁵ |
| (TOX3) | | | | | | | | | |
| 12p11 | reported | rs10771399 | 0.05 | G | [34] | 3,798 | 3,314 | 0.93 (0.84, 1.04) | 0.20 |
| (PTHLH) | novel | rs27633 | | C | | | | | |
| 5q11 | reported | rs889312 | 0.14 | C | [24] | 2,840 | 2,282 | 1.10 (1.01, 1.19) | 0.02 |
| (MAP3K1) | novel | rs16886113 | | C | | | | | |
| 9p21 | reported | rs1011970 | 0.00 | A | [34] | 3,807 | 3,316 | 1.09 (1.00, 1.18) | 0.05 |
| (CDKN2A/B) | novel | rs10965163 | | A | | | | | |
| 11p15 | reported | rs3817198 | | G | [24] | 3,266 | 2,636 | 1.14 (1.06, 1.23) | 8 × 10⁻⁴ |
| (LSP1) | | | | | | | | | |
| 8q24 | reported | rs13281615 | 0.00 | G | [24] | 3,338 | 2,723 | 1.06 (0.98, 1.13) | 0.13 |
| | novel | rs4733664 | | A | | | | | |
| 20q13 | reported | rs311498³ | 0.00 | A⁴ | [5] | 3,808 | 3,318 | 0.95 (0.84, 1.07) | 0.36 |
| | novel | rs13039229 | | C | | | | | |
| 6q25 | reported | rs9397435 | 0.01 | G | [35] | 3,809 | 3,316 | 1.14 (1.01, 1.27) | 0.03 |
| (ESR1) | novel | rs2253407 | | A | | | | | |
| 10q21 | reported | rs16917302 | 0.00 | C | [5] | 3,807 | 3,315 | 0.83 (0.75, 0.93) | 7 × 10⁻⁴ |
| (ZNF365) | novel | rs17221319 | | A | | | | | |
| 3p24 | reported | rs4973768 | | A | [24] | 3,370 | 2,783 | 1.10 (1.03, 1.18) | 6 × 10⁻³ |
| (SLC4A7, NEK10) | | | | | | | | | |
| 12q24 | reported | rs1292011⁴ | | G | [34] | 2,530 | 2,342 | 0.94 (0.87, 1.01) | 0.10 |
| 5p12 | reported | rs10941679⁴ | | G | [24] | 3,263 | 2,591 | 1.09 (1.01, 1.19) | 0.03 |
| 11q13 | reported | rs614367 | | A | [34] | 3,789 | 3,307 | 1.03 (0.95, 1.13) | 0.46 |
| 1p11 | reported | rs11249433 | | G | [35] | 3,423 | 2,827 | 1.09 (1.02, 1.17) | 0.02 |
| (NOTCH2) | | | | | | | | | |
| 17q23 | reported | rs6504950 | | A | [24] | 3,401 | 2,813 | 1.03 (0.95, 1.11) | 0.47 |
| (STXBP4, COX11) | | | | | | | | | |
| 19p13 | reported | rs8170 | | A | [5] | 3,665 | 3,086 | 0.98 (0.90, 1.07) | 0.66 |
| (MERIT40) | | | | | | | | | |
| 2q35 | reported | rs13387042⁴ | | G | [24] | 3,300 | 2,646 | 1.05 (0.98, 1.13) | 0.14 |
| 9q31 | reported | rs865686 | | C | [34] | 3,799 | 3,312 | 0.95 (0.89, 1.01) | 0.10 |
| 10q22 | reported | rs704010 | | A | [34] | 3,761 | 3,279 | 1.01 (0.95, 1.08) | 0.73 |
| (ZMIZ1) | | | | | | | | | |

| | | | | | iCOGS Results | | | | |
|---|---|---|---|---|---|---|---|---|---|
| Chr | Report | | | Minor | Affected | Unaffected | | Per Allele | |
| (Nearby Genes) | Status¹ | SNP | r² | Allele | N | N | MAF | HR (95% CI) | p-value² |
| 10q26 | reported | rs2981575 | 0.96 | G | 4,326 | 3,874 | 0.40 | 1.25 (1.18, 1.33) | 2 × 10⁻¹³ |
| (FGFR2) | novel | rs2420946 | | A | 4,328 | 3,877 | 0.39 | 1.27 (1.19, 1.34) | 2 × 10⁻¹⁴ |
| 16q12 | reported | rs3803662 | | A | 4,330 | 3,880 | 0.27 | 1.24 (1.16, 1.32) | 5 × 10⁻¹¹ |
| (TOX3) | | | | | | | | | |
| 12p11 | reported | rs10771399 | 0.05 | G | 4,330 | 3,880 | 0.11 | 0.89 (0.81, 0.98) | 0.02 |
| (PTHLH) | novel | rs27633 | | C | 4,252 | 3,841 | 0.39 | 1.14 (1.07, 1.21) | 4 × 10⁻⁵ |
| 5q11 | reported | rs889312 | 0.14 | C | 4,330 | 3,881 | 0.29 | 1.04 (0.98, 1.11) | 0.20 |
| (MAP3K1) | novel | rs16886113 | | C | 4.330 | 3,881 | 0.06 | 1.24 (1.11, 1.38) | 1 × 10⁻⁴ |
| 9p21 | reported | rs1011970 | 0.00 | A | 4,330 | 3,881 | 0.17 | 1.03 (0.95, 1.11) | 0.51 |
| (CDKN2A/B) | novel | rs10965163 | | A | 4,329 | 3,880 | 0.10 | 0.84 (0.77, 0.93) | 8 × 10⁻⁴ |
| 11p15 | reported | rs3817198 | | G | 4,316 | 3,870 | 0.33 | 1.11 (1.04, 1.18) | 9 × 10⁻⁴ |
| (LSP1) | | | | | | | | | |
| 8q24 | reported | rs13281615 | 0.00 | G | 4,248 | 3,810 | 0.43 | 1.03 (0.97, 1.09) | 0.31 |
| | novel | rs4733664 | | A | 4,329 | 3,879 | 0.41 | 1.10 (1.04, 1.17) | 2 × 10⁻³ |
| 20q13 | reported | rs311498³ | 0.00 | A⁴ | 4.330 | 3,880 | 0.07 | 0.95 (0.84, 1.06) | 0.31 |
| | novel | rs13039229 | | C | 4,326 | 3,877 | 0.21 | 0.90 (0.84, 0.97) | 5 × 10⁻³ |
| 6q25 | reported | rs9397435 | 0.01 | G | 4,330 | 3,881 | 0.08 | 1.12 (1.00, 1.25) | 0.03 |
| (ESR1) | novel | rs2253407 | | A | 4,330 | 3,881 | 0.47 | 0.92 (0.86, 0.98) | 5 × 10⁻³ |
| 10q21 | reported | rs16917302 | 0.00 | C | 4,330 | 3,881 | 0.11 | 0.88 (0.80, 0.98) | 0.01 |
| (ZNF365) | novel | rs17221319 | | A | 4,330 | 3,881 | 0.46 | 1.09 (1.02, 1.15) | 6 × 10⁻³ |
| 3p24 | reported | rs4973768 | | A | 4,322 | 3,875 | 0.49 | 1.09 (1.02, 1.15) | 7 × 10⁻³ |
| (SLC4A7, NEK10) | | | | | | | | | |
| 12q24 | reported | rs1292011⁴ | | G | 4.313 | 3.875 | 0.42 | 0.92 (0.87, 0.98) | 0.01 |
| 5p12 | reported | rs10941679⁴ | | G | 4,320 | 3,875 | 0.24 | 1.07 (1.01, 1.15) | 0.04 |
| 11q13 | reported | rs614367 | | A | 4,330 | 3,880 | 0.14 | 1.08 (1.00, 1.17) | 0.04 |
| 1p11 | reported | rs11249433 | | G | 4,328 | 3,881 | 0.40 | 1.05 (0.99, 1.12) | 0.10 |
| (NOTCH2) | | | | | | | | | |
| 17q23 | reported | rs6504950 | | A | 4,329 | 3,881 | 0.26 | 1.04 (0.97, 1.11) | 0.23 |
| (STXBP4, COX11) | | | | | | | | | |
| 19p13 | reported | rs8170 | | A | 4,327 | 3,876 | 0.19 | 0.98 (0.91, 1.06) | 0.62 |
| (MERIT40) | | | | | | | | | |
| 2q35 | reported | rs13387042⁴ | | G | 4,326 | 3,880 | 0.48 | 0.99 (0.93, 1.05) | 0.66 |
| 9q31 | reported | rs865686 | | C | 4,330 | 3,880 | 0.36 | 0.99 (0.93, 1.05) | 0.77 |
| 10q22 | reported | rs704010 | | A | 4.328 | 3.878 | 0.38 | 1.01 (0.95, 1.07) | 0.91 |
| (ZMIZ1) | | | | | | | | | |

¹Reporting status of the SNP is either previously reported or novel to this report.
²p-value was calculated based on the 1-degree of freedom score test statistic.
³rs311499 could not be designed onto the iCOGS array. A surrogate (r² = 1.0), rs311498, was included, however, and reported here.
⁴Stronger associations were originally reported for the SNP, assuming a dominant or recessive model of the ‘risk allele’.

TABLE 2
Breast cancer hazard ratios (HR) and 95% confidence intervals (CI) of novel breast cancer loci with P-values of
association <10⁻⁵ among BRCA2 mutation carriers

| SNP rs No. | | | Discovery Stage | | | | Stage 2 | | | |
|---|---|---|---|---|---|---|---|---|---|---|
| Chr. | | Affected | Unaffected | | | | Affected | Unaffected | | |
| (Nearby | | No. | No. | | | | No. | No. | | |
| Genes) | Genotype | (%) | (%) | MAF | HR (95% CI) | p-value¹ | (%) | (%) | MAF | HR (95% CI) |
| rs93485126 | CC | 390 | 248 | 0.39 | 1.00 | | 1,606 | 1392 | 0.35 | 1.00 |
| (TFAP2A | | (46.4) | (38.3) | | | | (46.0) | (43.0) | | |
| C6orf218) | CA | 368 | 299 | | 0.81 (0.67-0.96) | | 1515 | 1432 | | 0.92 (0.83-1.01) |
| | | (43.8) | (46.2) | | | | (43.4) | (44.3) | | |
| | AA | 82 | 100 | | 0.55 (0.42-0.74) | | 368 | 410 | | 0.72 (0.62-0.84) |
| | | (9.8) | (15.5) | | | | (10.5) | (12.7) | | |
| | per allele | | | | 0.76 (0.67-0.87) | 2.6 × 10⁻⁵ | | | | 0.87 (0.81-0.93) |
| rs619373 X | GG | 693 | 568 | 0.06 | 1.00 | | 2882 | 2784 | 0.07 | 1.00 |
| (FGF13) | | (75.8) | (87.8) | | | | (82.7) | (86.1) | | |
| | GA | 143 | 78 | | 1.43 (1.13-1.80) | | 583 | 439 | | 1.25 (1.10-1.43) |
| | | (15.7) | (12.1) | | | | (16.7) | (13.6) | | |
| | AA | 4 | 1 | | 2.01 (0.50-8.06) | | 21 | 11 | | 2.09 (1.09-4.03) |
| | | (8.5) | (0.1) | | | | (0.6) | (0.3) | | |
| | per allele | | | | 1.43 (1.15-1.78) | 3.0 × 10⁻³ | | | | 1.27 (1.12-1.44) |
| rs1845772 | GG | 520 | 368 | 0.25 | 1.00 | | 2104 | 1824 | 0.25 | 1.00 |
| (C2orf58) | | (61.9) | (56.9) | | | | (60.3) | (56.4) | | |
| | GA | 278 | 234 | | 0.86 (0.71-1.03) | | 1212 | 1231 | | 0.83 (0.75-0.92) |
| | | (33.1) | (36.2) | | | | (34.7) | (38.1) | | |
| | AA | 42 | 45 | | 0.67 (0.46-0.96) | | 174 | 179 | | 0.80 (0.64-0.99) |
| | | (5.0) | (7.0) | | | | (5.0) | (5.5) | | |
| | per allele | | | | 0.84 (0.73-0.97) | 1.5 × 10⁻² | | | | 0.86 (0.79-0.93) |

| SNP rs No. | | | Combined | | | | |
|---|---|---|---|---|---|---|---|
| Chr. | | | Affected | Unaffected | | | |
| (Nearby | | Stage 2 | No. | No. | | | |
| Genes) | Genotype | p-value¹ | (%) | (%) | MAF | HR (95% CI) | p-value¹ |
| rs93485126 | CC | | 1,996 | 1640 | 0.35 | 1.00 | |
| (TFAP2A | | | (46.1) | (42.3) | | | |
| C6orf218) | CA | | 1883 | 1731 | | 0.89 (0.82-0.97) | |
| | | | (43.5) | (44.6) | | | |
| | AA | | 450 | 510 | | 0.68 (0.59-0.78) | |
| | | | (10.4) | (12.1) | | | |
| | per allele | 5.2 × 10⁻⁵ | | | | 0.85 (0.80-0.90) | 3.9 × 10⁻⁸ |
| rs619373 X | GG | | 3575 | 3352 | 0.07 | 1.00 | |
| (FGF13) | | | (82.6) | (86.4) | | | |
| | GA | | 726 | 517 | | 1.29 (1.15-1.45) | |
| | | | (16.8) | (13.3) | | | |
| | AA | | 25 | 12 | | 1.99 (1.16-3.41) | |
| | | | (0.6) | (0.3) | | | |
| | per allele | 2.0 × 10⁻⁴ | | | | 1.30 (1.17-1.45) | 3.1 × 10⁻⁶ |
| rs1845772 | GG | | 2624 | 2192 | 0.25 | 1.00 | |
| (C2orf58) | | | (60.6) | (56.5) | | | |
| | GA | | 1490 | 1465 | | 0.83 (0.76-0.91) | |
| | | | (34.4) | (37.8) | | | |
| | AA | | 216 | 224 | | 0.77 (0.64-0.93) | |
| | | | (5.0) | (5.8) | | | |
| | per allele | 8.6 × 10⁻⁵ | | | | 0.85 (0.79-0.91) | 3.6 × 10⁻⁶ |

¹P-value was calculated based on the 1-degree of freedom score test

TABLE 3
Description of breast cancer affected and unaffected BRCA2 carriers
included in the final analysis of the COGs array SNPs

| | Affected | | Unaffected | |
|---|---|---|---|---|
| | (n = 4,330) | | (n = 3,881) | |
| Factor | N | % | N | % |
| Age at Censoring | | | | |
| <40 | 1,545 | 35.7 | 1,607 | 41.4 |
| 40-49 | 1,651 | 38.1 | 1,025 | 26.4 |
| 50-59 | 799 | 18.5 | 712 | 18.4 |
| 60+ | 335 | 7.7 | 537 | 13.8 |
| Ashkenazi Jewish Ancestry | | | | |
| No | 3,988 | 92.1 | 3,433 | 88.5 |
| Yes | 342 | 7.9 | 448 | 11.5 |
| BRCA2*617delT Mutation Carrier | | | | |
| Yes | 435 | 10.0 | 584 | 15.0 |
| No | 3,895 | 90.0 | 3,297 | 85.0 |
| Country of Residence | | | | |
| Australia | 288 | 6.7 | 200 | 5.2 |
| Austria | 123 | 2.8 | 77 | 2.0 |
| Canada | 153 | 3.5 | 150 | 3.9 |
| Denmark & Sweden | 158 | 3.6 | 198 | 5.1 |
| Finland | 66 | 1.5 | 55 | 1.4 |
| France | 491 | 11.3 | 209 | 5.4 |
| Germany | 365 | 8.4 | 198 | 5.1 |
| Iceland | 102 | 2.4 | 25 | 0.6 |
| Israel | 108 | 2.5 | 166 | 4.3 |
| Italy | 353 | 8.2 | 174 | 4.5 |
| South Africa | 93 | 2.1 | 53 | 1.4 |
| Spain | 328 | 7.6 | 293 | 7.5 |
| The Netherlands | 260 | 6.0 | 492 | 12.7 |
| United Kingdom & | 483 | 11.2 | 560 | 14.4 |
| Ireland | | | | |
| USA | 959 | 22.1 | 1,031 | 26.6 |
| Study | | | | |
| BCFR | 197 | 4.5 | 152 | 3.9 |
| BIDMC | 4 | 0.09 | 5 | 0.1 |
| BMBSA | 93 | 2.1 | 53 | 1.4 |
| BRICOH | 48 | 1.1 | 80 | 2.1 |
| CBCS | 46 | 1.1 | 49 | 1.3 |
| CNIO | 113 | 2.6 | 113 | 2.9 |
| COH | 65 | 1.5 | 42 | 1.1 |
| CONSIT TEAM | 263 | 6.1 | 137 | 3.5 |
| DFCI | 55 | 1.3 | 79 | 2.0 |
| DKFZ | 14 | 0.3 | 11 | 0.3 |
| EMBRACE | 478 | 11.0 | 547 | 14.1 |
| FCCC | 19 | 0.4 | 35 | 0.9 |
| GC-HBOC | 351 | 8.1 | 186 | 4.8 |
| GEMO | 523 | 12.1 | 226 | 5.8 |
| GOG | 152 | 3.5 | 161 | 4.1 |
| HCSC | 59 | 1.4 | 54 | 1.4 |
| HEBON | 260 | 6.0 | 492 | 12.7 |
| HEBCS | 66 | 1.5 | 55 | 1.4 |
| HVH | 34 | 0.8 | 25 | 0.6 |
| ICO | 122 | 2.8 | 102 | 2.6 |
| ILUH | 103 | 2.4 | 26 | 0.7 |
| INHERIT | 26 | 0.6 | 23 | 0.6 |
| IOVHBOCS | 90 | 2.1 | 37 | 1.0 |
| kConFab | 254 | 5.9 | 182 | 4.7 |
| MAGIC | 8 | 0.2 | 22 | 0.6 |
| MAYO | 80 | 1.8 | 61 | 1.6 |
| MCGILL | 12 | 0.3 | 15 | 0.4 |
| MSKCC | 121 | 2.8 | 97 | 2.5 |
| MUV | 123 | 2.8 | 77 | 2.0 |
| NCI | 22 | 0.5 | 61 | 1.6 |
| NICCC | 61 | 1.4 | 108 | 2.8 |
| OCGN | 65 | 1.5 | 112 | 2.9 |
| OSU CCG | 33 | 0.8 | 28 | 0.7 |
| OUH | 89 | 2.1 | 117 | 3.0 |
| SMC | 47 | 1.1 | 57 | 1.5 |
| SWE-BRCA | 23 | 0.5 | 31 | 0.8 |
| UCHICAGO | 25 | 0.6 | 12 | 0.3 |
| UCLA | 15 | 0.3 | 26 | 0.7 |
| UCSF | 16 | 0.4 | 11 | 0.3 |
| UKGRFOCR | 4 | 0.09 | 13 | 0.3 |
| UPENN | 134 | 3.1 | 105 | 2.7 |
| WCP | 17 | 0.4 | 54 | 1.4 |

TABLE 4
Quality control filtering steps for BRCA2 mutation carriers and SNPs on the COGs array

| | | Remaining | | | Remaining |
|---|---|---|---|---|---|
| | No. of | No. of | | No. of | No. of |
| Sample Data Cleaning Steps/Exclusion Reasons | samples | Samples | Data Cleaning Steps for SNPs | SNPs | SNPs |
| Total Eligible Samples on Manifest with Genotype Data | | 10,048 | Total SNPs on COGs Array | | 211,155 |
| Ineligible based on phenotypic data | 211 | 9,837 | Y chromosome SNPs | 79 | 211,076 |
| Self-reported non-CEU ethnicity | 531 | 9,306 | Call rate <95% | 4,446 | 206,630 |
| Incorrect gender based on genotype | 34 | 9,272 | HWE(stratified) P-value < 10⁻⁷ | 1,845 | 204,785 |
| Call rate <95% \| Heterozygosity: P-value < 10⁻⁶ | 300 | 8,972 | Monomorphic markers | 3,853 | 200,932 |
| >19% inferred non-CEU ancestry | 166 | 8,806 | Unreliable SNP genotypes | 1 | 200,931 |
| Discordant with previous CIMBA genotyping | 53 | 8,753 | SNPs with high discordance rate among | 23 | 200,908 |
| Consistent duplicate pairs (one sample excluded | 498 | 8,255 | known duplicates (list obtained from all | | |
| Inconsistent duplicate pairs (both samples excluded) | 44 | 8,211 | members of COGs consortia) | | |
| Totals After Filtering | 1,837 | 8,211 | | 10,439 | 200,908 |

TABLE 5
Breast cancer hazards ratios (HR) and 95% confidence intervals (CI) for all SNPs with P < 10⁻³
in a 500 Mb region around rs9348512 on 6p24 among BRCA2 mutation carriers

| | | | | | | Minor | | |
|---|---|---|---|---|---|---|---|---|
| | | | | Minor | r² with | Allele | r² | P- |
| SNP | Position | Type¹ | Major Allele | Allele | rs9348512 | Freq. | imputation | value² |
| rs9348512 | 10564692 | typed | C | A | 1.00 | 0.34 | 1.00 | 4.4 × 10⁻⁸ |
| rs9358529 | 10563215 | imputed | A | C | 0.86 | 0.31 | 0.96 | 8.2 × 10⁻⁷ |
| rs303067 | 10548212 | imputed | A | T | 0.71 | 0.40 | 0.95 | 1.9 × 10⁻⁶ |
| rs1348 | 10557244 | imputed | T | C | 0.51 | 0.21 | 0.96 | 1.0 × 10⁻⁵ |
| rs9366443 | 10565096 | imputed | C | T | 0.72 | 0.41 | 0.94 | 3.7 × 10⁻⁵ |
| rs9460713 | 10555412 | imputed | C | T | 0.49 | 0.20 | 0.96 | 4.9 × 10⁻⁵ |
| rs9466289 | 10555931 | imputed | T | C | 0.50 | 0.20 | 0.97 | 5.4 × 10⁻⁵ |
| 6-10546956 | 10546956 | imputed | A | AGG | 0.48 | 0.41 | 0.84 | 5.9 × 10⁻⁵ |
| rs9466290 | 10555941 | imputed | G | A | 0.49 | 0.20 | 0.96 | 6.4 × 10⁻⁵ |
| rs3911709 | 10559876 | imputed | G | A | 0.45 | 0.26 | 0.96 | 7.3 × 10⁻⁵ |
| 6-10557995 | 10557995 | imputed | GTAT | G | 0.49 | 0.20 | 0.95 | 7.4 × 10⁻⁵ |
| rs9295542 | 10565669 | imputed | A | G | 0.61 | 0.46 | 0.91 | 8.2 × 10⁻⁵ |
| rs6908107 | 10559449 | imputed | C | G | 0.61 | 0.46 | 0.98 | 8.8 × 10⁻⁵ |
| rs35076407 | 10563463 | imputed | T | C | 0.62 | 0.45 | 0.98 | 9.2 × 10⁻⁵ |
| rs602199 | 10554912 | imputed | C | G | 0.60 | 0.39 | 0.94 | 9.3 × 10⁻⁵ |
| rs7738545 | 10563318 | imputed | C | T | 0.62 | 0.45 | 0.99 | 9.7 × 10⁻⁵ |
| rs303074 | 10560093 | imputed | G | A | 0.61 | 0.46 | 0.98 | 1.0 × 10⁻⁴ |
| rs78113724 | 10543366 | imputed | G | A | 0.45 | 0.24 | 0.97 | 1.1 × 10⁻⁴ |
| rs303073 | 10560449 | imputed | A | G | 0.61 | 0.46 | 0.98 | 1.3 × 10⁻⁴ |
| rs4712668 | 10562060 | typed | G | T | 0.61 | 0.45 | 1.00 | 1.4 × 10⁻⁴ |
| rs75769093 | 10551514 | imputed | C | A | 0.54 | 0.47 | 0.90 | 1.5 × 10⁻⁴ |
| rs303070 | 10551187 | imputed | G | T | 0.56 | 0.48 | 0.92 | 1.9 × 10⁻⁴ |
| rs9393239 | 10550632 | imputed | C | T | 0.43 | 0.21 | 0.98 | 2.0 × 10⁻⁴ |
| rs303061 | 10538157 | typed | T | C | 0.41 | 0.24 | 1.00 | 2.1 × 10⁻⁴ |
| rs4097280 | 10561081 | imputed | G | A | 0.58 | 0.44 | 0.95 | 2.3 × 10⁻⁴ |
| rs4710998 | 10541811 | typed | A | G | 0.39 | 0.22 | 1.00 | 2.8 × 10⁻⁴ |
| rs6907578 | 10532198 | imputed | T | A | 0.53 | 0.43 | 0.86 | 3.7 × 10⁻⁴ |
| 6-10550552 | 10550552 | imputed | G | T | 0.55 | 0.47 | 0.90 | 3.9 × 10⁻⁴ |
| rs56365413 | 10540162 | imputed | C | T | 0.40 | 0.20 | 0.98 | 4.0 × 10⁻⁴ |
| 6-10545251 | 10545251 | imputed | GTTGTTGTT | G | 0.38 | 0.21 | 0.99 | 4.4 × 10⁻⁴ |
| rs303068 | 10549297 | imputed | A | C | 0.56 | 0.48 | 0.92 | 4.5 × 10⁻⁴ |
| rs6923826 | 10532295 | imputed | C | G | 0.50 | 0.42 | 0.86 | 4.8 × 10⁻⁴ |
| rs12175352 | 10545445 | imputed | T | C | 0.38 | 0.21 | 0.99 | 4.8 × 10⁻⁴ |
| rs303065 | 10541072 | imputed | C | T | 0.39 | 0.20 | 0.99 | 5.0 × 10⁻⁴ |
| rs303064 | 10540524 | typed | C | T | 0.38 | 0.20 | 1.00 | 5.2 × 10⁻⁴ |
| rs9295535 | 10547954 | typed | T | C | 0.40 | 0.21 | 1.00 | 6.2 × 10⁻⁴ |
| rs115262601 | 10526048 | imputed | A | C | 0.01 | 0.01 | 0.44 | 6.6 × 10⁻⁴ |
| rs6924202 | 10532454 | imputed | C | T | 0.56 | 0.39 | 0.80 | 6.7 × 10⁻⁴ |
| rs12526269 | 10536199 | imputed | T | A | 0.37 | 0.23 | 0.98 | 7.1 × 10⁻⁴ |
| 6-10546510 | 10546510 | imputed | GC | G | 0.48 | 0.44 | 0.94 | 7.9 × 10⁻⁴ |
| rs303063 | 10538964 | imputed | C | T | 0.38 | 0.20 | 0.99 | 8.7 × 10⁻⁴ |

¹Type indicates whether the SNP was genotyped or imputed.
²p-value was calculated based on the 1-degree of freedom score test

TABLE 6
Associations with SNPs at 6p24, FGF13 and 2p22 and breast and ovarian cancer risk using a competing
risk analysis model

| SNP rs No. | | No. breast | No. ovarian | Ovarian cancer | | Breast cancer | |
|---|---|---|---|---|---|---|---|
| Chr. | No. unaffected | cancer | cancer | HR (95% CI) | p-value | HR (95% CI) | p-value |
| rs9348512 | 3432 | 4310 | 468 | 0.98 (0.84, 1.13) | 0.74 | 0.84 (0.79, 0.90) | 8.7 × 10⁻⁸ |
| 6p24 | | | | | | | |
| rs619373 | 3432 | 4307 | 468 | 0.98 (0.73, 1.30) | 0.88 | 1.29 (1.15, 1.44) | 7.1 × 10⁻⁶ |
| Xq26 | | | | | | | |
| rs184577 | 3432 | 4311 | 468 | 1.01 (0.86, 1.19) | 0.89 | 0.85 (0.79, 0.92) | 1.4 × 10⁻⁵ |
| 2p22 | | | | | | | |

TABLE 7
>gnl|dbSNP|rs1348
rs = 1348|pos = 501|len = 1001|taxid = 9606|mol = “genomic”|class = 1|
alleles = “A/G”|build = 137|suspect = ?|GMAF = C: 2184:0.1603
GAAAGTATTG ATGGTGAGTA ACTTTGGTAA AAGAAGGTCC AGGCATGTGG CTCATGCCTA TAATCCCAGC
ACTTTGGGAG
GCCGAGGCAG GATAATTGCT TGAGGTCAGG AGTTCGAGAC TAGCCTAGGT AACATAGTAA AACCCTGCCC
CTACAAAAAA
TTAAAATATT AGCCAGGTGT GATGGTGCAC ATAGTCCTGT CATCCTAGCT ACTTGGGAGG CTGAGGCAGG
AGGATTGCTT
GAGCCCAGGA ATTTGAGGCT ACAGTGAGCT ATGATTGTGT CACTGCACTC CAGCCTAGGC AACCGAGTGA
GACTCCATTT
CTAAAAGAAA TTAATTAATT AATAAAAATG AAGAGAATGC TCAGCAGACT TCTGAATTCT TTGAGTGTTC
ACAAGAAAAA
TAAATTCATG TATCACATGT CCTATAAAAG AAAAAGGGGT TGGGGTTGCT TTTGCATATA AAGCAAGCTA
GTTTCAACCG
AACTCTCTGT CCACTGGAGA
R
AAGTGCTTGC CACAGAAAAC TGTTTTTCTG GTTCACTGAG GTATAATTGA CAAATAACAA GTACATATAT
CCAAGGTGCA
CAATGTGATG TTTTGATATA AGTATACACT GTGAAAGGAT TATTACAACC AATTTGTAGT AATTAACTTG
ATGAATTAAC
ATATTCATCA AGTATATAGT ACATTATTAT TAACTGTAGT CACCATGCTG TGCTTTCCAT CTCAGAAAAC
TTAGAGTATT
ACCCTAAAGT TAACTATGAC CATTTAGAAG TTTGCTTTAA AACACTGCTT TTCAAATTTT ACTGTGGAAA
CAAATCGCTT
GGGGATCTTG TTAAAATTTG GATTCTGAAT CAGTAGGTCT AGAGTGGGGC CTGAGATTCT GTCTTTCTGA
CAATCTCCCC
AGGTGACACC AATGCTGCTG GTCCCTGAAG CACGCTTTTG GTGGCAAGGT TTCCAGAGAG CTGAGGCTCC
TGTATTTCTT
TACAATCCAG ACATCAGTTT
>gnl|dbSNP|rs303061
rs = 303061|pos = 501|len = 1001|taxid = 9606|mol = “genomicm”|class = 1|
alleles = “A/G”|build = 137|suspect = ?|GMAF = C: 2184:0.234
CACCTGCTAC TGATACACAC TATAAACGTG GAAGATGATT TTCATTTTTG TAGTCATGAG CAGGATACTG
TATAATGTAT
AATTGTTGGA CATTAAAGAA AACAAACTCC TTCTTGTCTC CTTAGGCTCA GAGCCACTCA GACATTGGGA
AGCAAGTTTG
TCAAGATGAC AGAGAACCGA GGTAATGGAT TCGAGTGATG AAACAGGAAG TTCATTCATG AGTTTTTGGC
CACACCTCCA
AAGTGACGAC TTAGCCAGAA ATGGGATAAC TGGGTTTCCC TACTTCTCTT TTATCATCCT CAATGAGAGT
GACCAAATAT
TAGAGCTAGA TGGAACCTTA GTGAAAATCT GGCTACTCGT CCCGTCCCAC CAGCCTGCCA CCCATTTCAA
GTTTGAAGAG
ACAAAGACAC ATGGACCTTA TGTAATTACT GGGGATTACC CCAGGAGTCT GTGGCAAAAG TCAGCTTCTT
CCCTCCCTGC
TTCCCCGCCC TGTCTCTGGT
R
CTTTCTACCA ACACTGGGCT GTTTCTGTGA TCACACTTAA GCGTACCTAA CCTGCGAATG CTGTATAGAA
GGTGCTAATG
AACATGATTT AGCTTTAACA CTCAGTTTTC TAAAGGGACA CGTGGGGGCA GCAAATGTTT AGGCAAAAAC
AATTCCAGTT
CTAGCCTCTA CTGTCTACAT ATGTGTATAC ATTTGGGAAA CGTTTGGGAA AGGGATATTT GAGAGCTTCT
TTTTCTTTTT
TGTGGTTTAG TTATTTGATG ATATTGAGAT TGTTTCTGAG CCATGTGCTT CAACATCGGA TTGGGGATTT
CAGAAAAAGT
TTTAGTCACT GTGATTCCAT TTAGCTTCCA AATGTGTCTC TGCTAAGAGA CTTAAAAGCA CTCATAAATA
GCACGTGTGT
CTTCTTTGCA GTGTTTGCTA ATTTTGAGTC ACATCTTTTT AGAAAATCAT GAGATTTGGT GTCACAGAGA
CTGGAATAAA
TATAGTCAAA CTTATTGGTG
>gnl|dbSNP|rs303063
rs = 303063|pos = 501|len = 1001|taxid = 9606|mol = “genomic”|class = 1|
alleles = “A/G”|build = 137|suspect = ?|GMAF = T: 2184:0.2079
CACGTCTGTC ACTCTAAAGC CTGTTCTAAG ATCACATCCC CTCTGGCTCT CCCTAGATAC TTCTGATTAT
AGTTGGTAAT
CATGCCCATT AAATGTCTTT GGTTTCTTGG TACACAGAGA GAAGGTAAAT AATGGGTTCA TGATTTAGGT
AAGAATACAC
AAATATTTTT CACACTATAA ATCTGTTTTC TATTCAGTTA AATATTAGAC CATCTGGTAA TGGAGAAAAT
GTTTGAAAAT
CCACCTAGTT AACCTAAAAG TTTGCATTTC CTGCCTTTGG GGCTTTGAAT TTTTAAGTTA CATGTCTTCG
TAGATGGTGA
TCAGGATAAA ACTAATATCT CTCTTAGATG AATCAAAATT CACCCCCTTG GGCAAGGGAG GGACCCAGCC
TTTCTTTAAA
CATGTGGTTG CCTGAGAGTT GAACAAAACT GGAATAGGGA GAGAATGGCT TTGCCATGCT GATCATGGTA
TTGTGGAGAT
TTAAGAACTA GTGGCCAGGC
R
AGGTGGCTCA CGCCTGTAAT CCCAGCATTG TGAGAGGCCA AGGCAGGTGG ATCACTTGAG GCCAGGATTT
CGAGACCAGC
CCGGCCAACA TGATGACCCT GTTTTTACTA AAAATACAAA AATTAGCCGA GTGTGGTGGT GTGTGCCTGT
AGTCCCAACT
GAGGCATGAG ACTTGCTTCA ACCCAGGAGG CGGGGGTTGC AGTGAGCCGA GATTGTGCCA CTGCACTCCA
GCCTGGGTGA
CAGAGTGAGA CTCTGTCTCA AAAAAAAAAA AAAAAAAAAC TAGTCAGAGC TGTTGAGGTG TTGAGGCACC
TGCTACTGAT
ACACACTATA AACGTGGAAG ATGATTTTCA TTTTTGTAGT CATGAGCAGG ATACTGTATA ATGTATAATT
GTTGGACATT
AAAGAAAACA AACTCCTTCT TGTCTCCTTA GGCTCAGAGC CACTCAGACA TTGGGAAGCA AGTTTGTCAA
GATGACAGAG
AACCGAGGTA ATGGATTCGA
>gnl|dbSNP|rs303064
rs = 303064|pos = 501|len = 1001|taxid = 9606|mol = “genomic”|class = 1|
alleles = “A/G”|build = 137|suspect = ?|GMAF = T: 2184:0.2092
ATAGTGAGAT TCTGCCTCTA AAACAAAACA AAATAAAAGA CAAAACAACT AATTCTGTTT TTTAAAAAAA
AAAAACACAC
AACAGCAAAC TTGACTATAA AGATTATTGC TGGGCACGGT GGCTCAGGCC TGTAATCCCA GCACTTTGGG
AGGCTGAGGC
GGGTGGATCA CCTGAGGTCA GGAGCTCAAG ACCAGCCTGG CCAACATGGT GAAACCCTGT CTGTACTAAT
AATACAAAAA
ATTAGCCGGG CATGGTGGTG CATGCCTGCA ATCCCAGCTA CTCGGGAGGA TGAGGCAGGA GAATCACTTG
AACCTGGGAG
GTAGAGGTTG CAGTGAGCCG AGACTGCGCC ACTGCACTCC AGCCTGGGCA ACAAGAGCAA AACTCCGTGT
CCAAAAAAAA
AAAAAAAAAA AAAGATTATT ATATATAATC ATTCAAGGCC TGTATGACTC AGTTCCCTTA GAAAAATGTC
ATAATTTTTA
TATTACTGAA TATTATTGGC
R
TTATTTGTGT AGCCCACTTA AGTGAAGTCA ATAACATGAT TAAGTGGCAT ATTATCTTCA TGTCAGTCAA
ACGTTATTTG
GATTTTATAA GTTAGGGTGA GATACAAATA AGTGAAAATA CTTTTTCTAA TGAATAATGA TGAATCTAAA
ATAGGATTGA
CTTGGCTGGG CATAGTGGCT CGTGCTTGTA ACCCCAACAC TTTGGGAGGC TGAGGCAGTA GGATTACTTG
AAACCAGGAG
TTTGAGACCA GCCTCGGCAA CAAAGGGAGA ACTCTTCTCT AATAAAAATA AGAATAAAAA ATTAGCCAGG
TGTGGCAATG
TTCACCTGTG GTCCCAGCTA CTTGGGAAGC TGAGGCAGGA GGATCGTTGG AGCACAGGAG TTCAAGACTG
CAGTTAGCGG
TGACTGCACT CCAGCCTGGG CAATAGAGCA AGACCCTGTC TCTAAAAAAA AAATAATAAT AAATAGGACT
GGCTCGCATA
TGTATGCAAC TATTTTGTTA
>gnl|dbSNP|rs303065
rs = 303065|pos = 501|len = 1001|taxid = 9606|mol = “genomic”|class = 1|
alleles = “C/T”|build = 137|suspect = ?|GMAF = T: 2184:0.2083
ACTGAGTCAT ACAGGCCTTG AATGATTATA TATAATAATC TTTTTTTTTT TTTTTTTTTT TGGACACGGA
GTTTTGCTCT
TGTTGCCCAG GCTGGAGTGC AGTGGCGCAG TCTCGGCTCA CTGCAACCTC TACCTCCCAG GTTCAAGTGA
TTCTCCTGCC
TCATCCTCCC GAGTAGCTGG GATTGCAGGC ATGCACCACC ATGCCCGGCT AATTTTTTGT ATTATTAGTA
CAGACAGGGT
TTCACCATGT TGGCCAGGCT GGTCTTGAGC TCCTGACCTC AGGTGATCCA CCCGCCTCAG CCTCCCAAAG
TGCTGGGATT
ACAGGCCTGA GCCACCGTGC CCAGCAATAA TCTTTATAGT CAAGTTTGCT GTTGTGTGTT TTTTTTTTTT
AAAAAACAGA
ATTAGTTGTT TTGTCTTTTA TTTTGTTTTG TTTTAGAGGC AGAATCTCAC TATGTTGCCC AGGCTTATTT
TGCACCACTG
GCCTCAAGCA GTCCTCCCAG
Y
TCTGCCAAAT TTTAGACACC TGGACTTGGA AACCCATGAG AAGTTGGCCA GCGCTTCCTT TTGCATTTAT
GCAGAGCAAT
GGTAAACGTC AGCAGCAAAT TTACAATCAA TCTTATTTTC CAGTGTCTCC AGAGATTTGA TTGTTTTGCT
TATATGGCTT
ATGGCAATAC TTACCTCCAA TGTCTGATTT TTCAAAAAAA TTTCATCTAA TCCTTTGTGC AATGGTTTAC
ATCTAATTTT
TTTTGTTCTG AGATGTAGAC AGCTATTAAG AACTGATCTT TGCACAGTAA ATTTTTCCTA TTCTTAGTCA
TTATCCTTAG
GTGAGGCAAC ATGGTGCCAT TCAGTTATTC AGCAAATTCT CACATCTTCC GTGTGCCATG CATTGTTACA
GGTTTTGAGC
CTAGAACCAT GAAAACAAAA GACAAAAATC TCTGCCCTTG TGGACTTTGT ATTCCAGAGG AGAGAAAATA
AACAAGGTAA
GTAAAATATA TAGTATGGTA
>gnl|dbSNP|rs303067
rs = 303067|pos = 501|len = 1001|taxid = 9606|mol = “genomic”|class = 1|
alleles = “A/T”|build = 137|suspect = ?|GMAF = A: 2184:0.4986
TGTGTTCATT ATGACCACTG GGAAGATGAG TTTCACCCTT CACATGACTA TATGGTGAGA ACATGTTTCT
CAGCTGAGAC
AAGATTTCAG GAAAGTTTGC AAGAACCGAG AGAAAATGGA AGAAAATGAA ATATTTGTTC TTCAGAGTCA
CCAGTTTTAT
TATATGCCTG GACTCTGTCA CTTATGTCAA TAAATTTACA AATGCAAAAT ACACATTTAA TTCCAGCGTG
GTAGCATACA
CCTGTAGTCC TAGATACTCA GGAGGGTGAG TATCTAGGAC TACAGGTGTG TGCTACCACG CTTGAACTCA
GGAGTTCAAG
GCCAGCCTGG ACAACACAGG GAGACCCCCT CTCTAAATGT ATATACACAC ATACACACAC ACACACACAC
ACACACACAT
TCAAACATTG GAATCAGATG TCCTGGACAA AATGCTCAAA TCAGCTGAAC ACTTTGGAAT GCTTAACTTT
TCTTTTTTTT
AGATGTTTAT TTATTTATTT
W
TTTTGTTTTT TATTTTATTA TTATTATACT TTAAGTTTTA GGGTACATGT GCGCAATGTG CAGGTTTGTT
ACATATGTAT
ACATGTGCCA TGTTGGTGTG CTGCACCCAT TAACTCGTCA TTTAGCATTA GGTATATCTC CAAATGCTAT
CCCTCCCCCC
TCCCCCCACC CCACAACAGT CCCCGGAGTG TGATGTTCCC CTTCCTGTGT CCATGTGTTC TCATTGTTCA
ATTCCCACCT
ATGAGTGAGA ACATGCGGTG TTTGGTTTTT TGTCCTTGTG ATAGTTTGCT GAGGATGATG GTTTCCAGTT
TCATCCATGT
CCCTACAAAG GACATGAACT CATCATTTTT TATGGCTGCA TAGTATTCCA TGGTGTATAT GTGCCACATT
TTCTTAATCC
AGTCTATCAT TGTTGGACAT TTGGGTTGGT TCCAAGTCTT TGCTATTGTG AATAGTGCCG CAATAAACCT
ACGTGTGCAT
GTGTCTTTAT AGCAGCATGA
>gnl|dbSNP|rs303068
rs = 303068|pos = 501|len = 1001|taxid = 9606|mol = “genomic”|class = 1|
alleles = “A/C”|build = 137|suspect = ?|GMAF = A: 2184:0.4148
CACTGACTTC CACAATGGTT GAACTAGTTT ACGGTCCCAC CAACAGTGTA AAAGTGTTCC TATTTCTCCA
CATCCTCTCC
AGCACCTGTT GTTTCCTGAC TTTTTAATGA TCACCATTCT AACTGGTGTG AGATGGTATC TCATTGTGGT
TTTGATTTGC
ATTTCTCTGA TGGCCAGTGA TGATGAGCAT TTTTTCATGT GTTTTTTGGC TGCATAAATG TCTTCTTTTG
AGAAGTGTCT
GTTCATATCC TTCGCCCACT TTTTGATGGG GTTGTTTATT TTTTTCTTGT AAATTTGTTT GAGTTCATTG
TAGATTCTGG
ATATTAGCCC TTTGTCAGAT GAGTAGGTTG CAAAAATTTT CTCCCATTTC GTAGGTTGCT TGTTCACTCT
GATGGTAGTT
TCTTTTGCTG GAATGCTTAA CTTTTCTTTC CTGCAAGAGG AAGACTGGGA GATTGAGAGG TGTGCCCAGG
GTGTCAGCTC
AGTGCCTGGT AGAGGCAGGT
M
ACATGAGCTT TGAGCCCTGG CCTGTGGATT GTGTTGTGCT TGGACTGGCC TGTTACGCCA TCTGCTTGCT
TCTGCCTGAA
ATTCCTGTAA CACAAGATAA AACCTCTCAT TCTGCAATTT ACCAATAAAC CTTATAAAAT CTGAGGCTAA
GCCTTGTGAA
ATGGCAAACC CTAGAAAGCC AGAGAGCTGG CATGCGGTAG TTCAGCAGTG GCTGCAGGAA ATAGAAGGAA
AGGGAGTGAG
GAGACAGGGT GCAAGCTGGA ATGTGATCAC AGGGGAGGGG GATCTCTGGA CTATGGGGGA ATGACGGGCA
GCTTGAGGCC
CTGAGATGAA AGACACTCCC TTTAAGAAAA ACGTCTGTGA CTCAGAGCCA CAAGGCGTGT CAGGAGAAAG
TTCTGATGAA
ACTGCATTCC ATTCCTGGAG GAATACGCAT TGCCATTGAA GTACAAACTC TAAAATGATG TAGGATTAAT
AATTAAGGCA
ACACTAGTAG TGGATGGTGG
>gnl|dbSNP|rs303070
rs = 303070|pos = 501|len = 1001|taxid = 9606|mol = “genomic”|class = 1|
alleles = “G/T”|build = 137|suspect = ?|GMAF = G: 2184:0.4038
TTCTTTTAAA ACTAAACATA TACAGTTGTC CATCAGTATC TGCAGGGGAC TGGTTCCAGG TCTCCCTGCA
AGTACCAAAA
TCACAGATGC TCAGGAATCT GATATAAAGC AGGGTAGTAC TTGCATATAA CCCACACACA TCCTCCTGTA
TACTTTAAAT
CATCTCTAGA TTACTTATAA TACCTAGTAC AAGGTGAAGA CTATGTAAAC AGTTGTTATA CTGTATTTAA
AAAAATAATT
TCCACTTTCA TTTTAGATTC AGGTGGTACA TATGCAGGTT TGTTATATAA GTGTATTGCA TGATGCTGAG
GTTTGGAGTT
CAATTGATCC CATCACTCAG ATAGTGAGCA TAATTATACT GTATACAGTT TAGGAAATAA TAACAAGAAA
AAAGTCTATA
CATATTCCAT ACAGACATGA TCATCCTTTC CTCCCACCCC TTGATTTTTT ATTGAATTCA TGAATATTTG
CTGAGTCCAT
GAATGTGGAA CCCACGGATA
K
GGAAGGCTGA CTGCACCTAC ATTATGACCT GGCAATTCCA CACCTAGGTT ACTCACCCGG GAGAAATAAA
AGCATATGTC
CTCAAAGAGG CTTGTTCAAA AATGTCCATA GCTTTATTCA TAAATAACTG AAAGCTGAAA ACAACCAATA
GGAGAATGAA
TAAACTAACT GTGGTAAATT CAGACAATGA AATACTACAC AATAAAAAAG GGAGGAACCG GCTGGGCGCG
GTGGCTCACA
CCTGTAATCC CAGCACATTG GGAGGCCGAG GTGGGTGGAT CACCTGGGGT CAAGAGTTCG AGACCAGCCT
GGCCAACATG
GTGAAACCCC ATCTCTACTA AAAATACAAA AATAGTCAGG TGTGGTGGCA CGCACCTCCA ATCCCAGCTA
CTCGAAAGGC
TGAGGCAGGA GAATCAGCTT GAATCCAGGA GGCAGAGGTT GCAGTGAGCT GAGATTGTGC CACTGCACTC
CAGCCTGGGT
GACTCTGTCT CAAAAAAAAA
>gnl|dbSNP|rs303073
rs = 303073|pos = 501|len = 1001|taxid = 9606|mol = “genomic”|class = 1|
alleles = “C/T”|build = 137|suspect = ?|GMAF = A: 2184:0.4139
ATGAACTAGG TTTTGAACTC CCCCATGCAT GACCCCTGCC TGTGGGGTCT GCCCTAGATG GATAATAAAT
AGGTCATTAT
CAGTCTCATT TCAGTGTCCA CAGTGGAGAG ATTTTATTCT TCCCCTTGCT CTGGAACTGG CCCCTTTTCT
CCTCCAAATC
CCAATCTTGG CCTAGAATTT TGAACTCTGC TTAGAATTCC AAATTGCCAC ATATATATGT CAGGAGATCA
GACAGAGTTA
GCTGAGCAGG GAACAAGGCC GTGCTTTTCA GAAGTATGTA GGTCTGCTTC ACAAGAATAT GCCATTAACA
ATATGGACAA
GGCTCACCAT AAATTTATGA GTGAAACAAC TTATTCCAAC TGCTCTCATG CCTGGCTTTA TACAGTCATC
TACTTGTCCT
TCCCTGGGCC CAGCCAATGC TGCTCCCCCT TTAACAACTG CTTCTGAATG TCCCTGTGGT GTGGGCCAGA
AAGGAGACTC
TCTTCTTCCC CAAATCCACC
Y
GCAGTATGGC AGAAACTAGA ATTCATGGTC CTCTCCCAAC CCATGCCCAC TTCCTTCTGC CACTTAAAGA
AAACACCCAT
AAAGGGTGGG AAGAGAGAGC GTAACAGCAA GGTCTGTGCA TTCCCAGAGA TGTGATGCAA GGGGTGTGGG
AGGCATGGCA
CTGCTTGACT CACGCTGGAG AGCGGGCACT TGGCCTGGCT TTCAGAGGAA ATGCTCCTTG GAATGCGGTC
GGCCCCGGCT
GCACCCACGC CTGTGAGGGA GGGGCTTATG TGTCTGGCAC ATCATAGGTG GCTCCTGGGG TTTGCCATGA
GTCTCAGCAC
AGCAGACCTG AGAGGAAAAA AATACAGACT GAACGCGTTT CTTCTATTCT CTCACCCAAC ACAGAAGACT
TCTGTGGCCG
CATGTGTGGG GTTTTTTTTC CCCACATACC AAGAAGCAAA CAATTCTTCT GTGGACTCTA GCCGAGACTC
TTCCAATTCA
ATTCAATTCA GACACTAGCT
>gnl|dbSNP|rs303074
rs = 303074|pos = 501|len = 1001|taxid = 9606|mol = “genomic”|class = 1|
alleles = “C/T”|build = 137|suspect = ?|GMAF = G: 2184:0.4245
CAACTGCTCT CATGCCTGGC TTTATACAGT CATCTACTTG TCCTTCCCTG GGCCCAGCCA ATGCTGCTCC
CCCTTTAACA
ACTGCTTCTG AATGTCCCTG TGGTGTGGGC CAGAAAGGAG ACTCTCTTCT TCCCCAAATC CACCTGCAGT
ATGGCAGAAA
CTAGAATTCA TGGTCCTCTC CCAACCCATG CCCACTTCCT TCTGCCACTT AAAGAAAACA CCCATAAAGG
GTGGGAAGAG
AGAGCGTAAC AGCAAGGTCT GTGCATTCCC AGAGATGTGA TGCAAGGGGT GTGGGAGGCA TGGCACTGCT
TGACTCACGC
TGGAGAGCGG GCACTTGGCC TGGCTTTCAG AGGAAATGCT CCTTGGAATG CGGTCGGCCC CGGCTGCACC
CACGCCTGTG
AGGGAGGGGC TTATGTGTCT GGCACATCAT AGGTGGCTCC TGGGGTTTGC CATGAGTCTC AGCACAGCAG
ACCTGAGAGG
AAAAAAATAC AGACTGAACG
Y
GTTTCTTCTA TTCTCTCACC CAACACAGAA GACTTCTGTG GCCGCATGTG TGGGGTTTTT TTTCCCCACA
TACCAAGAAG
CAAACAATTC TTCTGTGGAC TCTAGCCGAG ACTCTTCCAA TTCAATTCAA TTCAGACACT AGCTACCTAG
AGATAGTGTA
AGAAGGCACA GGTTGAGGAC TCATTCCCCA AGACCACCCC TCACTCCTGA TGCCAACTGC AAGCTCCACG
TTGTTTTATC
TGTGCATCTG ACTGGCTATA AATCAGGGGT CCTATGGCCC CCTCCGGTGG CCCTTCCTTG GGTTTGATTA
ATATGCTAGA
GCAGCTCACA GAACTCAGGG AAACACCGAT TTATTATAAA GGATATTACA AAGGATAAAG ATTAATAGAT
GCATAGGGTA
AAGTATGGGG GAAGGCAGAA GCAGTTTCCA CGTCCTTCCC AAGCACCACA CCCTCCAGGA ACCTCCACGT
GGTCAGCTAC
CCAGAAGGTC TCCAAACATG
>gnl|dbSNP|rs602199
rs = 602199|pos = 501|len = 1001|taxid = 9606|mol = “genomic”|class = 1|
alleles = “C/G”|build = 137|suspect = ?|GMAF = G: 2184:0.4505
GCAGATGGGG CCTTTGGGAG GTGACTAGGA CATGAGGGTG GAGCACTTGT GAGTGGGATT AGTGCCCTTA
CAGAGAGACT
GCAGAGAGCT CCCTTGCCCC TTCCACCATG TGAGGACACA GGGAGAAGAT GGCTGTCTGT GAACTGGGAA
ACAGGCCCTC
ATCAGACACT GAGTCAGCTG AGGTATTGAT CTTGGACTTC CCAGCCTTCA GAACTGTGAG AAATAAATTT
CTGTTTAAAA
GACACCCAGT TTCAGGTATT TTTGCCATAG TAGCCCAAAC AGACTAACAG TGGATATAAT TTTGTTGAGC
CAGTTATAAA
ATCCAAGTCC AAGTCAAAAC TGCAGGCTGA TATTAGCTGG GCATGGTGGT GCATGCCTGT AGTTCCAGCT
ACTCCAGAGG
CGGAGGTGGG AGGATGGCTT GGGCCCCAGA GGTCAAGGCT GCTGTGAGTG GTGATCACAC CAATGCACAC
CAGCCTGGGG
GACAGAGCAA GACCCTGTCT
S
AAAAACACAA AAATACAAAA AACTTGCAGG CTCCAAAACT ACCTGAACTT CAACCTACCT TCTTTCAATA
CAATGCCCAG
AACATAGTCT CTCTCAATGA CCCAGTCAAG CTTAAAAAAA AAAAAAAGAA AAAAAAAGCA TCACCACTTC
TCTCTTCAAA
ATCTAGCCTT GATATATATT TTGAGGGGAT GCATTCTGAA GTGTGTCAGC ATGATACATC GTATCATCTG
CAACTTACTT
TCAAATGGCT TAGGAAAAAA ATATGGTATC TGCTGTCTAT TTATCTACCT ACCTATCTAT CAAGAGATAA
GCAAACATGA
TGAAATAATA ACAGTTGTTA AATCCATGTG AGGGGTATTT GGCTGTTTGT GTGCTATTTT TCAGCTTTTC
TGATTGAGCT
TACTTTTTTA AAAAGGCAGG AAAAAGTATG TGGCCTTGAC TGTGAAGACA GACAAGAAAC AGCTGAGCCC
CTCTTGTTTC
TCGATACATC AAAAATGCGG
>gnl|dbSNP|rs3911709
rs = 3911709|pos = 501|len = 1001|taxid = 9606|mol = “genomic”|class = 1|
alleles = “C/T”|build = 137|suspect = ?|GMAF = A: 2184:0.3173
ACACCCATAA AGGGTGGGAA GAGAGAGCGT AACAGCAAGG TCTGTGCATT CCCAGAGATG TGATGCAAGG
GGTGTGGGAG
GCATGGCACT GCTTGACTCA CGCTGGAGAG CGGGCACTTG GCCTGGCTTT CAGAGGAAAT GCTCCTTGGA
ATGCGGTCGG
CCCCGGCTGC ACCCACGCCT GTGAGGGAGG GGCTTATGTG TCTGGCACAT CATAGGTGGC TCCTGGGGTT
TGCCATGAGT
CTCAGCACAG CAGACCTGAG AGGAAAAAAA TACAGACTGA ACGCGTTTCT TCTATTCTCT CACCCAACAC
AGAAGACTTC
TGTGGCCGCA TGTGTGGGGT TTTTTTTCCC CACATACCAA GAAGCAAACA ATTCTTCTGT GGACTCTAGC
CGAGACTCTT
CCAATTCAAT TCAATTCAGA CACTAGCTAC CTAGAGATAG TGTAAGAAGG CACAGGTTGA GGACTCATTC
CCCAAGACCA
CCCCTCACTC CTGATGCCAA
Y
TGCAAGCTCC ACGTTGTTTT ATCTGTGCAT CTGACTGGCT ATAAATCAGG GGTCCTATGG CCCCCTCCGG
TGGCCCTTCC
TTGGGTTTGA TTAATATGCT AGAGCAGCTC ACAGAACTCA GGGAAACACC GATTTATTAT AAAGGATATT
ACAAAGGATA
AAGATTAATA GATGCATAGG GTAAAGTATG GGGGAAGGCA GAAGCAGTTT CCACGTCCTT CCCAAGCACC
ACACCCTCCA
GGAACCTCCA CGTGGTCAGC TACCCAGAAG GTCTCCAAAC ATGGTTCTTT TGGGCTTTGA TGGAGGCTTT
ATTATGTAGG
CATGATTGAT TAAACTCTTG GTCATTGGTG ATTAACTTTA CTTCAGCCCC TCTTCCCTTC CCAGAGGTTG
GGGGATGAGG
CTGAAAGCCC CAACCCTCTA ACCATGGCTT AGCCTTTCCC ATGACCAGTC TCCGTTCTGA AGCTACCGAT
GGGCTGCCGG
CCATCTGTCA ACTCATCAGC
>gnl|dbSNP|rs4097280
rs = 4097280|pos = 501|len = 1001|taxid = 9606|mol = “genomic”|class = 1|
alleles = “C/T”|build = 137|suspect = ?|GMAF = A: 2184:0.4789
GCTGGTCTCA AATTCCTGAC CTGAAATGAT CCGCCCGCCT CGGCCTCCCA AAGTGCTGGG ATTACAGGTG
TAAGCCACCA
CGCCTGGCCT GCCCATCTCT TTTTCACTTG AGCCAAAGCT ATTTCTAGGA AGGCAGTGGC ATTTCCTGAG
CTAAAATCAT
TTTCCCATTC CTGAGTCACA TTTCACATGG TCCCAGAGGT GAATTTAGTG GATTACATTT TAAAAAACAA
ACAAAAACCT
CAGAGCCACA CATAGACCAG GTTTTGCCTT CTTCCTCTCC AACTCCCACT ATTCCTTCCT TTGCACGTTT
GCTGAGCCAT
ACGGAAGTGC ATGGCCAACA GAGAAGAAAA AGGTTGATTA GTAGTAAAGA AGCCCTGCTG TTGCCTTGAA
TGTCAGCACG
TGCACACACA CACACAGGTG CGCGCACACA CGGGCACACA CAGGTACGCG CACACACACA GGTGCAAGCA
CACAGGTGCG
CGCACACACA CAGATGCGCG
Y
GCACACACAG GTACGCGCAC ACACACTTTT ATACCTGTCC ATTGCCAGTT TCTTTTGGTT CTTCAGGATT
GCCCTTAGTG
TTCGTTTTCA TCTGATGTTG CCTAGACCAA AATCCTGGAG GAAAGAACCT GATGAACTAG GTTTTGAACT
CCCCCATGCA
TGACCCCTGC CTGTGGGGTC TGCCCTAGAT GGATAATAAA TAGGTCATTA TCAGTCTCAT TTCAGTGTCC
ACAGTGGAGA
GATTTTATTC TTCCCCTTGC TCTGGAACTG GCCCCTTTTC TCCTCCAAAT CCCAATCTTG GCCTAGAATT
TTGAACTCTG
CTTAGAATTC CAAATTGCCA CATATATATG TCAGGAGATC AGACAGAGTT AGCTGAGCAG GGAACAAGGC
CGTGCTTTTC
AGAAGTATGT AGGTCTGCTT CACAAGAATA TGCCATTAAC AATATGGACA AGGCTCACCA TAAATTTATG
AGTGAAACAA
CTTATTCCAA CTGCTCTCAT
>gnl|dbSNP|rs4710998
rs = 4710998|pos = 501|len = 1001|taxid = 9606|mol = “genomic”|class = 1|
alleles = “A/G”|build = 137|suspect = ?|GMAF = G: 2184:0.2056
TTTTTTGTTC TGAGATGTAG ACAGCTATTA AGAACTGATC TTTGCACAGT AAATTTTTCC TATTCTTAGT
CATTATCCTT
AGGTGAGGCA ACATGGTGCC ATTCAGTTAT TCAGCAAATT CTCACATCTT CCGTGTGCCA TGCATTGTTA
CAGGTTTTGA
GCCTAGAACC ATGAAAACAA AAGACAAAAA TCTCTGCCCT TGTGGACTTT GTATTCCAGA GGAGAGAAAA
TAAAcAAGGT
AAGTAAAATA TATAGTATGG TAGATAATGA ATGGTATGGG CTGAAGGAAA AACATAAACA AAGACGTTAG
GTAGTGCTGA
GAGGGAGGGT GGTTTGCAGT TGAGATAAGG ATGTCTCCAA GGATATGGTG GCATCTGCAT TACCAGAGAA
ACATTCCAGG
CAGAGAAACC AGTGAGTGCA AAGGCCCTGA GGCAGAAGCA TGGCTGGCTT GTGTGGTAGG CATGAGTGAG
CTAAGGACAG
AGGAGCTACA GAGGACGCCA
R
AGAGAAATTG AGAATTGAAG GGAGTTGGGG GATGGAGCTG TTGGTACCGA AGGGAGACAT TATAAAGACC
TTAGCTTGGC
TGGGCACTGT GGCTCATGCC TGTAATCCCA GCACATTGGG AAGCCGAGGC AGGTGGATCA CCTGAGGTCA
AGAGTTCGAG
ACCAGCCTGG CCAACATGGG GAAACCCCAT CTCTACTAAA AATACAAAAA TTAGCCGGGC GTGGTGGCGT
GCACCTGTAA
TCCCAGCTAC TTGGGAGGCT GAGGCAAGAG AATCGCTTGA ACCCGGGAAG TGGAGGTTGC AGTGAGCCAA
GATCACACCA
CTGCACTGCA GTCTGGGCAA CAAGAGTGAA ACTCCATCTC AAAAAAAAAA AAGACCTTAG CTTTTCCTCT
GAGAGCAGAA
ACTTTGGAGG GGCTTGAGTC AAGCAATGGC GTGATCTGCT TCGTATCTTA ACAGAGTCAC CTTGGCTGCT
CGGTCAGGGC
AGGGGGACAA GTGTGGAGGC
>gnl|dbSNP|rs4712668
rs = 4712668|pos = 501|len = 1001|taxid = 9606|mol = “genomic”|class = 1|
alleles = “G/T”|build = 137|suspect = ?|GMAF = T: 2184:0.494
AGGTCAGGAA TTTGAGACCA GCCTGGCTGA CATGGTGAAA CCCGGTCGCT ACTAAAAATT CCAAAAATTA
GCCGGGCATC
GTGGCGGGCA CCTGTAGTCC CAGCTACTCA GGAGGCTGAG TCTGGAGAAT GGCTTGAACC CAGAAGATGG
AGGCTGCAGT
GAGCCAAGAT CGTGCCACTG CACTCCAGCC TGGGCAACAG AGCGAGACTC CATCTTAAAA AAAAAAGAAA
AGAAAGAAAA
AGAAATGGGC AAGACAAAAC CAAGTTTAAG AATGCAAGTT TATTACTTAT GAAATTATAG ATTGCAGGAA
CCAGAGACTT
AAGTTTCTCC AGGCAGTAGT GTATATTATC AGGATGAGGT AAGGAACACC AGACCAACAG GGCAGACAGG
TCTGATGAAG
GAAAGGGACC TGAAGGTCAT TCTGAATCCA GTGGAGCTCT AAGTAGGTCA ACTTTTGGCC TCCTCAGACC
AACATCCCTT
TGTGGTGACT CAAGACCAAT
K
TCTACCTCAG GGTCAGGCTG GTTTGGTCAA CCACCTCCAG TATGGCTGAC TTAGTTTTCA AATTCAGCCA
CAAGGATCAC
ATTGAGGAGT CTTTTTTTCA GAGACAGGGT CTTGCCCTGT TGCCCAGGCT GGAGTGCAAT GGTACAATCA
TAACTCACTG
CAGCCTCGAC CTCTTGGGCT CAAGCAATCC TCCTGCCTCA GCCTCCTGAG TAGCTGGGAC TACAGACACA
CACCACCATG
CCCAGCTATT TATTTTATTT TTGTAGAAAT GGGTCTTGCT ATGTTGCCCA GGCTGGTCTT CAACTCCTGA
TCCTCCCATC
TTGGCCTCCC AAAGTGCTGG GATTACAGGC ATGAGCCACC TTGCCCAGCT GATCTTGAGG AGTAGTTCTT
TCTTTGCTCT
GAAGCCCCAT GGCTTCATTT GAATGTATGA CTAAGCCCCT GTTAATGGTA ACCAGTGAAA TGAATTATTA
TTTTTTTTAT
CATTTTTATT TTTTTTTGAG
>gnl|dbSNP|rs6907578
rs = 6907578|pos = 501|len = 1001|taxid = 9606|mol = “genomic”|class = 1|
alleles = “A/T”|build = 137|suspect = ?|GMAF = T: 2184:0.4373
TTGCACTTCT GTTTACAAAG CAATCCTGCC ATCAAAAGAG GAACAAAATC ACCACTTATC ACACCCTGTC
ATAAAGTAAT
CTGCCCTGGA GGGAAACCCT TGTGGAGAAC TGCTGTGTGT GTGTGTGTGT GTGTGTGTGT GTGTGTGTGT
GTGTGTTGGG
GGAGGGGGGA TGGAGGAAAG GGGGATTCTC CCAACTCTTC TGCAGAGTAA ATCGCTGGAA CGCGTGGTGT
CCCAACCGGC
CTGGAAAGAC CGAGAGTACC ATGAGCTGTG AAGCTGGGGT GTGACAGGGA TGCCCGTCCA GGGCTGGCAA
GAGTGCAGAA
TGGCTCTCTT GGATCTTTGG AATAGGCACA TCTGCAGACC CCGCTCCAAT GTTTACTTTC CTAGCGCCTT
CGAAGATACT
CCCAAGGGCC CCCAAAATAG ATCAGCAAAA AGTGTTGGGG GTGGGGGGAG TGAAAAAGCC AGTTCTTGAA
GACTGTAAGG
TCCCCTTTCG CATCTCAGCA
W
CTGGAGTGTG CAGGGAATTC CTGACCAGTG GTTTTGCTCC CTCCAATCCC TTGCCTCCCC CCTCCCATGT
TATGCACTTG
TTCTTGGAGA GATGGACGTT AAAGAAGCGT CAAGCAGTTC TCACTGCAAA TAAATGGTGC AGAAATAAGA
GAGAGAGGAT
GAAAGCCTAG GAAGTTATAA GTGATCCTGA CCCGACCCAG CCACCAGGGG GTTATCTCTT TCCAGGTCCT
GCCTTGTGTA
GAGTGAGGTG ATAAACGCTT TAGGCAGCCA AATCCAAGCA CAGCTGGGTG CCTGGCGGGG ATGGGGTGGG
GGTGGTCCTA
TGTGGTGCCT CTGCCTCTGG AGTTACCTTT AGGAAAGGTC AAGAGAACTA TCCTCCCCTC CATGTCTGCT
GAAAAGGGGC
TATTTTGCTA GTCTTGTTAT CAGTAATTCA CCACTTAATA TAACCAGGTT TTAGGTTTGT ATATGAGCGA
TCCTGGACAT
CCAATACCAT CCCCCCAGTT
>gnl|dbSNP|rs6908107
rs = 6908107|pos = 501|len = 1001|taxid = 9606|mol = “genomic”|class = 1|
alleles = “C/G”|build = 137|suspect = ?|GMAF = C: 2184:0.4277
TGTCCTACTG CCCTTCCCAG CACCTGGCTT TAATTGGGGC TGCTCTGGGC CACTTTGCCA GGAATCCTGA
AGTTGATTTG
TAGGGAACAG GGATTGAGTG ACCGGGCCCT ACCTCCGCTC CCCAAAAACA ATGTCCTGTT CTCATGTGCT
GGCCCACCTC
CTCCCCAGGA CCTGGGTCCC TACGCTGAAC ACTGAGGTGG CTTTTGCTCA GCTAGTCTCC AAGACAGCAC
GAGCCTATTT
TGCCTATATT GGTAAGAGTA ATGGAGCTGT TCATTCCAGT TATCTTTCAC TGGACTGAAA GGATTGGCTT
AAAAAATTAC
TGTACCCTAC TGCGATATTG AAAAATATAT ATTTCATCTT CCACCTTGTT TCCCTGTGTA CAACTCCTAA
ATTCCTTGGA
ATCTCCAAAG TGATGTCTTT TTTGTGTGCT GATGAGTTGA CAGATGGCCG GCAGCCCATC GGTAGCTTCA
GAACGGAGAC
TGGTCATGGG AAAGGCTAAG
S
CATGGTTAGA GGGTTGGGGC TTTCAGCCTC ATCCCCCAAC CTCTGGGAAG GGAAGAGGGG CTGAAGTAAA
GTTAATCACC
AATGACCAAG AGTTTAATCA ATCATGCCTA CATAATAAAG CCTCCATCAA AGCCCAAAAG AACCATGTTT
GGAGACCTTC
TGGGTAGCTG ACCACGTGGA GGTTCCTGGA GGGTGTGGTG CTTGGGAAGG ACGTGGAAAC TGCTTCTGCC
TTCCCCCATA
CTTTACCCTA TGCATCTATT AATCTTTATC CTTTGTAATA TCCTTTATAA TAAATCGGTG TTTCCCTGAG
TTCTGTGAGC
TGCTCTAGCA TATTAATCAA ACCCAAGGAA GGGCCACCGG AGGGGGCCAT AGGACCCCTG ATTTATAGCC
AGTCAGATGC
ACAGATAAAA CAACGTGGAG CTTGCAGTTG GCATCAGGAG TGAGGGGTGG TCTTGGGGAA TGAGTCCTCA
ACCTGTGCCT
TCTTACACTA TCTCTAGGTA
>gnl|dbSNP|rs6923826
rs = 6923826|pos = 501|len = 1001|taxid = 9606|mol = “genomic”|class = 1|
alleles = “C/G”|build = 137|suspect = ?|GMAF = C: 2184:0.4455
CCTTGTGGAG AACTGCTGTG TGTGTGTGTG TGTGTGTGTG TGTGTGTGTG TGTGTGTGTT GGGGGAGGGG
GGATGGAGGA
AAGGGGGATT CTCCCAACTC TTCTGCAGAG TAAATCGCTG GAACGCGTGG TGTCCCAACC GGCCTGGAAA
GACCGAGAGT
ACCATGAGCT GTGAAGCTGG GGTGTGACAG GGATGCCCGT CCAGGGCTGG CAAGAGTGCA GAATGGCTCT
CTTGGATCTT
TGGAATAGGC ACATCTGCAG ACCCCGCTCC AATGTTTACT TTCCTAGCGC CTTCGAAGAT ACTCCCAAGG
GCCCCCAAAA
TAGATCAGCA AAAAGTGTTG GGGGTGGGGG GAGTGAAAAA GCCAGTTCTT GAAGACTGTA AGGTCCCCTT
TCGCATCTCA
GCATCTGGAG TGTGCAGGGA ATTCCTGACC AGTGGTTTTG CTCCCTCCAA TCCCTTGCCT CCCCCCTCCC
ATGTTATGCA
CTTGTTCTTG GAGAGATGGA
S
GTTAAAGAAG CGTCAAGCAG TTCTCACTGC AAATAAATGG TGCAGAAATA AGAGAGAGAG GATGAAAGCC
TAGGAAGTTA
TAAGTGATCC TGACCCGACC CAGCCACCAG GGGGTTATCT CTTTCCAGGT CCTGCCTTGT GTAGAGTGAG
GTGATAAACG
CTTTAGGCAG CCAAATCCAA GCACAGCTGG GTGCCTGGCG GGGATGGGGT GGGGGTGGTC CTATGTGGTG
CCTCTGCCTC
TGGAGTTACC TTTAGGAAAG GTCAAGAGAA CTATCCTCCC CTCCATGTCT GCTGAAAAGG GGCTATTTTG
CTAGTCTTGT
TATCAGTAAT TCACCACTTA ATATAACCAG GTTTTAGGTT TGTATATGAG CGATCCTGGA CATCCAATAC
CATCCCCCCA
GTTCCCCAGC GCCACTCCTG GACATTCTAG ACACCAGCGA GGCTTCTTCT GCAGCCATTC CTAATGTAGC
AGATCCATTT
TGGGGGGAGT CTGGATGCAG
>gnl|dbSNP|rs6924202
rs = 6924202|pos = 501|len = 1001|taxid = 9606|mol = “genomic”|class = 1|
alleles = “C/T”|build = 137|suspect = ?|GMAF = T: 2184:0.4771
TACCATGAGC TGTGAAGCTG GGGTGTGACA GGGATGCCCG TCCAGGGCTG GCAAGAGTGC AGAATGGCTC
TCTTGGATCT
TTGGAATAGG CACATCTGCA GACCCCGCTC CAATGTTTAC TTTCCTAGCG CCTTCGAAGA TACTCCCAAG
GGCCCCCAAA
ATAGATCAGC AAAAAGTGTT GGGGGTGGGG GGAGTGAAAA AGCCAGTTCT TGAAGACTGT AAGGTCCCCT
TTCGCATCTC
AGCATCTGGA GTGTGCAGGG AATTCCTGAC CAGTGGTTTT GCTCCCTCCA ATCCCTTGCC TCCCCCCTCC
CATGTTATGC
ACTTGTTCTT GGAGAGATGG ACGTTAAAGA AGCGTCAAGC AGTTCTCACT GCAAATAAAT GGTGCAGAAA
TAAGAGAGAG
AGGATGAAAG CCTAGGAAGT TATAAGTGAT CCTGACCCGA CCCAGCCACC AGGGGGTTAT CTCTTTCCAG
GTCCTGCCTT
GTGTAGAGTG AGGTGATAAA
Y
GCTTTAGGCA GCCAAATCCA AGCACAGCTG GGTGCCTGGC GGGGATGGGG TGGGGGTGGT CCTATGTGGT
GCCTCTGCCT
CTGGAGTTAC CTTTAGGAAA GGTCAAGAGA ACTATCCTCC CCTCCATGTC TGCTGAAAAG GGGCTATTTT
GCTAGTCTTG
TTATCAGTAA TTCACCACTT AATATAACCA GGTTTTAGGT TTGTATATGA GCGATCCTGG ACATCCAATA
CCATCCCCCC
AGTTCCCCAG CGCCACTCCT GGACATTCTA GACACCAGCG AGGCTTCTTC TGCAGCCATT CCTAATGTAG
CAGATCCATT
TTGGGGGGAG TCTGGATGCA GGTGTGTGTG ATCCAGCCTG AATTTGAGAC TCTCAGTTTC TTTAACACCA
GCTTGAAAAG
TCTGCAATCA CTAGCCCTGA GAGAGTACTT TGGTTCCTAA TGGGATATCC TGAGTCAGGG TGGCTGAAAG
AGCTACCAGT
TTACCTTGTA CATGGCAGGC
>gnl|dbSNP|rs7738545
rs = 7738545|pos = 501|len = 1001|taxid = 9606|mol = “genomic”|class = 1|
alleles = “C/T”|build = 137|suspec = ?|GMAF = T: 2184:0.4899
GCGTGAGCCA CCGAGCTTGG CCAGTGAAAT GAATTATTAA ACATTATTAA CCTTGGTATT GATATATCAA
AATTAATTTA
CTAAAGAGTG TTGTGGCCCA CACCTGTAAT CCCAGCACTT TGGGAGGCCA AGGTGAGAGG ATTTCTTGAG
CCCAGGAGTT
CAAGACCAGC CTGGGCAACA AGGCCAGACC CCATCCCTAC AAAAAATTTT TTTAAAAAAA TAGCCACCAG
GTATGGTGGT
GCACGCCTGT GGTCTCAACT GCTTGGGAGG CTGAGGCAGG AGGAATGTTT GAGCCCAGAA GGTCGGGGCT
GCAGCAGTGA
GCTGTGATCA CACCACTGCA TTCCAGCTTG GGTAACAGAG TGAGATCTTT TCTCAAACAA ACAAACAAAC
AAACAAAA.AA
AAGATTTGGA ATCAATATCC TAGCAAGACT CTGGGTTGCA ACTTTGCAAA TCTTCTGCTG TGCACGTTTG
TTGTTGTTGT
TGAGACACAG TCTCGCTCTG
Y
TGCCCAGGCT GGAGTGCAGT GGCACAATCA TTGCTCACTG AAACCTCGAC CTCCTGGACT CAAGCATTCC
TCCCGCGTCA
GCCTCCCAAG TCTCTGGGAC TATAGGCGTG CACCACCACG CCTGGCTAAT TAAATAAAAA ATTGTGGGTG
CCAGGCGCGG
TGGCTCACGC CTATAATCCC AGCACTTTGG GAGGGCGAGG AGGGTGGATC ACGAGGTCAA GAGATTGAGA
CCATTCTGGC
CAACCTGGTG AAATCCAGTC TCTACTAAAA TTACAAAAAT TAGCCGGGCG TGGTGGCGCA TGCCTGTAGT
CCCGGCTACT
CGGGAGGCTG AGGCAGGAGA ATCACTTGAA GCCGGGAGGC AGAGGTTGCA GTGAGCCGAT ATTGTACCAC
TGCACTCCAG
CCTGGCGACA GAGCAAGACT TCGTTTCAGA AAAAGAAAAA AAATTTTTTT TTTTTGTAGA AACAGAGTCT
TTCTATGTTG
CCCAGGCTGA TCGCAAACTC
>gnl|dbSNP|rs9295535
rs = 9295535|pos = 501|len = 1001|taxid = 9606|mol = “genomic”|class = 1|
alleles = “C/T”|build = 137|suspect = ?|GMAF = C: 2184:0.2074
TTAAAGGCGT GCACCACCAT GCATGGCTAA TTTTTGTATT TTTAGTAGAG TCAGGATTTC GACGTGTTGG
CCAGGCTGGT
CTCGAACTCC TGACCTCAGG TAATCCACCC TCCTCGGCCT CCCAAAGTAC TAGGATTACA GGCATGAGCC
ACATTACCTG
GCCACATTTA AGCTTTTTAA CTAAAAGTTT ATTGGGAGAA TAAAGTGGAG GGCAGTTAAA ATCCCTCTAG
TGGAAGAAAA
GACCTGGACA ATATGAGCTG TGTTCATTAT GACCACTGGG AAGATGAGTT TCACCCTTCA CATGACTATA
TGGTGAGAAC
ATGTTTCTCA GCTGAGACAA GATTTCAGGA AAGTTTGCAA GAACCGAGAG AAAATGGAAG AAAATGAAAT
ATTTGTTCTT
CAGAGTCACC AGTTTTATTA TATGCCTGGA CTCTGTCACT TATGTCAATA AATTTACAAA TGCAAAATAC
ACATTTAATT
CCAGCGTGGT AGCATACACC
Y
GTAGTCCTAG ATACTCAGGA GGGTGAGTAT CTAGGACTAC AGGTGTGTGC TACCACGCTT GAACTCAGGA
GTTCAAGGCC
AGCCTGGACA ACACAGGGAG ACCCCCTCTC TAAATGTATA TACACACATA CACACACACA CACACACACA
CACACATTCA
AACATTGGAA TCAGATGTCC TGGACAAAAT GCTCAAATCA GCTGAACACT TTGGAATGCT TAACTTTTCT
TTTTTTTAGA
TGTTTATTTA TTTATTTATT TTGTTTTTTA TTTTATTATT ATTATACTTT AAGTTTTAGG GTACATGTGC
GCAATGTGCA
GGTTTGTTAC ATATGTATAC ATGTGCCATG TTGGTGTGCT GCACCCATTA ACTCGTCATT TAGCATTAGG
TATATCTCCA
AATGCTATCC CTCCCCCCTC CCCCCACCCC ACAACAGTCC CCGGAGTGTG ATGTTCCCCT TCCTGTGTCC
ATGTGTTCTC
ATTGTTCAAT TCCCACCTAT
>gnl|dbSNP|rs9295542
rs = 9295542|pos = 501|len = 1001|taxid = 9606|mol = “genomic”|class = 1|
alleles = “A/G”|build = 137|suspect = ?|GMAF = G: 2184:0.494
ATATCTCATG TATTGGGTAT CTACTATGTG CCAAGCCTTT TACTAGGTGC TTTACACACA CATTTATATT
GTGCCACTTA
AAACTTATAG TAACTCTAAA AGATGATGGC TGGGTGCACT GGCCCACACC TGTAATCCCA GCAAGTTGGG
AGCCCAAGGT
GGGAGGATGG CTTGAGGCCA GGAGTTTGAC ACCAGCAGGG AGAACATAGC AAGACCTCAT CTCTACAAAA
TTAAAAAAAA
AAATACAAAA ATTAGCCGAG TGTGGTGGTG CACACCTGTA GTCCCAGGTA CTTGGGAGGT TGAGGTGGGA
GGATCACTTG
AGCCCAGGAG GTTGAGGCTG CAGTGAGCTA TGATTGTACC ACTGCGCTCC AGCTCAGATA ACAGAGCCAG
ACCCTATCTC
TAAAATTTAA ATAAATAAAT AAATAAATAA ATAAATAAAT AAATAAATAA TGTCTATTAT CCCCATTTTG
AAAAAAAAAA
AAAATCTGAG AGAAGGCCAG
R
CACCATGGCT CAAACCTGTA GACAGAGACG GGCAGAAGGC TTGGTCAAGA GTTCGAGACC AGCCTGGCTA
ACATGGTGAA
AACCCCTGCC TCTACTAAAA ATACAAAAAT TAGCCAAGTA TGATGGTGGC GCCTGCTATC CCAGCTACTT
GGGAGGCTGA
GGCAGAATTG CTTGAACCCG GGAGGCAGAG GTTGCAGTGA GCTGAGATCA CACCACTGCA CTCCAGCCTG
GGTGACAGAG
CGAGTATCCA TCTCAAAAAA AAAAAAAAAA AAAAGCCTGA GAGAGAAATC AAGCAATATG CCCCAAATTA
CACTACTAGC
AAATAACAAA ATCAAAATTC AATCCCAAGT CTCAATTTTC TTTTCAAATT CTTCACTTAC TATATCCGTT
TCCTGTTGCT
GCTGTAGCAC GTTACTGTGC ACTTTCTCAC GGTGCAGGAG ATGAGAAGTC TGAAATCAAG GAGTCAGCAA
GGCCACAGTC
CCTCCAGAGG CTCCAGGAGA
>gnl|dbSNP|rs9348512
rs = 9348512|pos = 501|len = 1001|taxid = 9606|mol = “genomic”|class = 1|
alleles = “A/C”|build = 137|suspect = ?|GMAF = A: 2184:0.2944
ACTAATGCCT CTTCAAATGG AGGGTTTTGT TTGTTTGTTT GTTTATGGGA ATTTTAAGTA ATTTTCAGTG
CCTGAGAATG
TTCTCCATAA AACCTGTAAC AAAACACATA ATAATGGTTC CAGTGAAAAT AGTTATCTCA AAGTTGGATT
GGATTTGAAA
TTCTAAATAC CCTATGACTA GGGTATCAAA ATTTAAGGTT TGGTCAAATG TAACTTTTTA GGTGTCTTGT
GTATGGTACA
AGTTTGAAAG TGTTTATGTG CACTACCTGT TCCATTCATC ATATCTACCC ATATCTGTAT CACTTAAAAT
GAATACTTTT
AGGTTTATTA AAAAGTAACA CTTCAAGCAA GCAAATGGAA ATTATTTTGC AGTAACTACA AATAATAGAG
AAGTATTAAC
ATAGAGTTGT GGGCCATGAC CTAGTGGGTT GTTTGTCACT GTTTATTTTC TGCCATTTCC TAGGGGTGAA
TTGCATCCTG
TACTGTTTAC AGCCTTATCT
M
CAACTTTTGC AGAGTCAAGA ATTTAAAAGC AGCAGGGCTT GGTGGCCCAT GCCTGTAATC CCAATATTTT
GGGAGGCCGC
AGCGGGAGGA TCACTTGAGG CCAGGAGTTC CAGACCTCCC TGGGCAACAT GATGAGACCA CATCTCTACA
GAAAAATTAG
CTGGGCATGC TGGCATGTGC CTGTAGTCCC AGCTCCTCAA GAGGCTGAGG TGGGAGGATC ACTTGAGCCC
AGGAGGTCAA
GGCTGCAGTG AGCTATGATC ACACCAATGC ACTCTAGCCT GGGGACACAG TGAGACCCTG TCTCAAAAAA
AAAAAAAAGA
GGAATTTAAA AGCATTTACT ACTATCTAGT GTACTATATA CTTATTATTA GTTTATTTTT GGTCTCCCTC
ACCAGAATGT
AGGCTCCTTA AAGGCAGTAG TATTGCACAT AATAGGGACT AATATACCTT TATTGAATTA ATTAATGAGG
GCCAAGATAT
CTCATGTATT GGGTATCTAC
>gnl|dbSNP|rs9358529
rs = 9358529|pos = 501|len = 1001|taxid = 9606|mol = =genomic”|class = 1|
alleles = =A/C”|build = 137|suspect = ?|GMAF = C: 2184:0.2816
ATTTTTAGTA GAGATGGGGT TTCACCACGT TGGCCAGGCT AGTCTCGATC TCCTGACCTC GTGATCCTCC
CACCTCGGCC
TCCCCAAAGT GCTGGGATTA CAGGCGTGAG CCACCGAGCT TGGCCAGTGA AATGAATTAT TAAACATTAT
TAACCTTGGT
ATTGATATAT CAAAATTAAT TTACTAAAGA GTGTTGTGGC CCACACCTGT AATCCCAGCA CTTTGGGAGG
CCAAGGTGAG
AGGATTTCTT GAGCCCAGGA GTTCAAGACC AGCCTGGGCA ACAAGGCCAG ACCCCATCCC TACAAAAAAT
TTTTTTAAAA
AAATAGCCAC CAGGTATGGT GGTGCACGCC TGTGGTCTCA ACTGCTTGGG AGGCTGAGGC AGGAGGAATG
TTTGAGCCCA
GAAGGTCGGG GCTGCAGCAG TGAGCTGTGA TCACACCACT GCATTCCAGC TTGGGTAACA GAGTGAGATC
TTTTCTCAAA
CAAACAAACA AACAAACAAA
M
AAAAGATTTG GAATCAATAT CCTAGCAAGA CTCTGGGTTG CAACTTTGCA AATCTTCTGC TGTGCACGTT
TGTTGTTGTT
GTTGAGACAC AGTCTCGCTC TGCTGCCCAG GCTGGAGTGC AGTGGCACAA TCATTGCTCA CTGAAACCTC
GACCTCCTGG
ACTCAAGCAT TCCTCCCGCG TCAGCCTCCC AAGTCTCTGG GACTATAGGC GTGCACCACC ACGCCTGGCT
AATTAAATAA
AAAATTGTGG GTGCCAGGCG CGGTGGCTCA CGCCTATAAT CCCAGCACTT TGGGAGGGCG AGGAGGGTGG
ATCACGAGGT
CAAGAGATTG AGACCATTCT GGCCAACCTG GTGAAATCCA GTCTCTACTA AAATTACAAA AATTAGCCGG
GCGTGGTGGC
GCATGCCTGT AGTCCCGGCT ACTCGGGAGG CTGAGGCAGG AGAATCACTT GAAGCCGGGA GGCAGAGGTT
GCAGTGAGCC
GATATTGTAC CACTGCACTC
>gnl|dbSNP/rs9366443
rs = 9366443|pos = 501|len = 1001|taxid = 9606|mol = “genomic”|class = 1|
alleles = “C/T”|build = 137|suspect = ?|GMAF = T: 2184:0.3837
AGTTGTGGGC CATGACCTAG TGGGTTGTTT GTCACTGTTT ATTTTCTGCC ATTTCCTAGG GGTGAATTGC
ATCCTGTACT
GTTTACAGCC TTATCTCCAA CTTTTGCAGA GTCAAGAATT TAAAAGCAGC AGGGCTTGGT GGCCCATGCC
TGTAATCCCA
ATATTTTGGG AGGCCGCAGC GGGAGGATCA CTTGAGGCCA GGAGTTCCAG ACCTCCCTGG GCAACATGAT
GAGACCACAT
CTCTACAGAA AAATTAGCTG GGCATGCTGG CATGTGCCTG TAGTCCCAGC TCCTCAAGAG GCTGAGGTGG
GAGGATCACT
TGAGCCCAGG AGGTCAAGGC TGCAGTGAGC TATGATCACA CCAATGCACT CTAGCCTGGG GACACAGTGA
GACCCTGTCT
CAAAAAAAAA AAAAAGAGGA ATTTAAAAGC ATTTACTACT ATCTAGTGTA CTATATACTT ATTATTAGTT
TATTTTTGGT
CTCCCTCACC AGAATGTAGG
Y
TCCTTAAAGG CAGTAGTATT GCACATAATA GGGACTAATA TACCTTTATT GAATTAATTA ATGAGGGCCA
AGATATCTCA
TGTATTGGGT ATCTACTATG TGCCAAGCCT TTTACTAGGT GCTTTACACA CACATTTATA TTGTGCCACT
TAAAACTTAT
AGTAACTCTA AAAGATGATG GCTGGGTGCA CTGGCCCACA CCTGTAATCC CAGCAAGTTG GGAGCCCAAG
GTGGGAGGAT
GGCTTGAGGC CAGGAGTTTG ACACCAGCAG GGAGAACATA GCAAGACCTC ATCTCTACAA AATTAAAAAA
AAAAATACAA
AAATTAGCCG AGTGTGGTGG TGCACACCTG TAGTCCCAGG TACTTGGGAG GTTGAGGTGG GAGGATCACT
TGAGCCCAGG
AGGTTGAGGC TGCAGTGAGC TATGATTGTA CCACTGCGCT CCAGCTCAGA TAACAGAGCC AGACCCTATC
TCTAAAATTT
AAATAAATAA ATAAATAAAT
>gnl|dbSNP|rs9393239
rs = 9393239|pos = 501|len = 1001|taxid = 9606|mol = “genomic”|class = 1|
alleles = “C/T”|build = 137|suspect = ?|GMAF = T: 2184:0.1625
ATGAGAACTC ACTCACTATC ATGAGGACAG CAAGGGGGAA ATTCTCCCCC ATGAGCCAAT CACCTCCCAC
CAGGTCCCTC
CCCCAACATT AGGAATTACA ATTTGCATGA GATTTGTGTG GCCACACGGA GCCAAACCAT ATCACATTGG
TCAACCTATA
TAGTAATGTT TTTCTTAATC TAAATGTATA CTAAGTTAGT GTTTTATCGA TTTAAAAATA CATCTTGAAA
AGGATTTTGC
AATTTACTTT TTTTTTTTTT TTGTGAGACA GAGTCTCACT CTTGTCCCCC AGGCTGGAGT GTAGTAGCGT
GATCTTGGCT
CGCTGCAATC TCTGCCTCCC AGGTTCAAGC AATTCTCCTG CCTCAGCCTC CTGAGTAGCT TGGATTACAG
GCGCCTGCCA
CTACTCCCGG CTAATTTTTT GGTATTTTTA GTAGAGACAG GGTTTCACCA TGTTGGCCAG GCTGGTTTCA
AACTCCTGAC
CTCAAGTGAT CCGCCCACCT
Y
GGCTTCCCAA AGTGCTAGGA TTACAGACGT GAGCCACCAT GCCCAGCCCA CAATTTCTTT TAAAACTAAA
CATATACAGT
TGTCCATCAG TATCTGCAGG GGACTGGTTC CAGGTCTCCC TGCAAGTACC AAAATCACAG ATGCTCAGGA
ATCTGATATA
AAGCAGGGTA GTACTTGCAT ATAACCCACA CACATCCTCC TGTATACTTT AAATCATCTC TAGATTACTT
ATAATACCTA
GTACAAGGTG AAGACTATGT AAACAGTTGT TATACTGTAT TTAAAAAAAT AATTTCCACT TTCATTTTAG
ATTCAGGTGG
TACATATGCA GGTTTGTTAT ATAAGTGTAT TGCATGATGC TGAGGTTTGG AGTTCAATTG ATCCCATCAC
TCAGATAGTG
AGCATAATTA TACTGTATAC AGTTTAGGAA ATAATAACAA GAAAAAAGTC TATACATATT CCATACAGAC
ATGATCATCC
TTTCCTCCCA CCCCTTGATT
>gnl|dbSNP|rs9460713
rs = 9460713|pos = 501|len = 1001|taxid = 9606|mol = “genomic”|class = 1|
alleles = “C/T”|build = 137|suspect = ?|GMAF = T: 2184:0.1584
CAGACAGGGT CTTGCTCTGT CCCCCAGGCT GGTGTGCATT GGTGTGATCA CCACTCACAG CAGCCTTGAC
CTCTGGGGCC
CAAGCCATCC TCCCACCTCC GCCTCTGGAG TAGCTGGAAC TACAGGCATG CACCACCATG CCCAGCTAAT
ATCAGCCTGC
AGTTTTGACT TGGACTTGGA TTTTATAACT GGCTCAACAA AATTATATCC ACTGTTAGTC TGTTTGGGCT
ACTATGGCAA
AAATACCTGA AACTGGGTGT CTTTTAAACA GAAATTTATT TCTCACAGTT CTGAAGGCTG GGAAGTCCAA
GATCAATACC
TCAGCTGACT CAGTGTCTGA TGAGGGCCTG TTTCCCAGTT CACAGACAGC CATCTTCTCC CTGTGTCCTC
ACATGGTGGA
AGGGGCAAGG GAGCTCTCTG CAGTCTCTCT GTAAGGGCAC TAATCCCACT CACAAGTGCT CCACCCTCAT
GTCCTAGTCA
CCTCCCAAAG GCCCCATCTG
Y
TAATACCATC ACCTTGGGGA TTAGAATACC AGTGTATGAA TTTGGAGGGG AGATAAGCAT TCAGTCCATT
GCACCCTTAT
TTCCAAGGCC CAGGGATAAC GCTGAGCTCC TCTGTGGGTG AAGCACATTC AGCTATAAAA CAGTATCTTA
AGATTTTCTT
CTCGAGTTAG ATTTGGTACG TAGATAACGA CCTTTAACTA TTTGCATCTA TGCAGCTTTT ACTTCCACCT
CCTCAACCCA
CTGTCTACAA TTCTCACATA GAATTAAGAA TAATTTTGCA TAGCAGATAA TTGGCTGAGC ATGCGGTATT
CTTTTGACCC
ATTCAAGTGA ATAAAATACT GTATAGGAAC ACTGTCACAA TTTAAATGAA ATTAAATTTC TTGCCCCTTT
TCCTCCCCCG
ACCATTTAGT CTTTGGGTAG CAACAGAGAT CACTAACATG ATATAACAAT TAATGTAGTT TTGTTTCAGG
GCATTAATTT
GATACAAATT GTAATTCTGT
>gnl|dbSNP|rs9466289
rs = 9466289|pos = 501|len = 1001|taxid = 9606|mol = “genomic”|class = 1|
alleles = “C/T”|build = 137|suspect = ?|GMAF = C: 2184:0.1593
GATTAGAATA CCAGTGTATG AATTTGGAGG GGAGATAAGC ATTCAGTCCA TTGCACCCTT ATTTCCAAGG
CCCAGGGATA
ACGCTGAGCT CCTCTGTGGG TGAAGCACAT TCAGCTATAA AACAGTATCT TAAGATTTTC TTCTCGAGTT
AGATTTGGTA
CGTAGATAAC GACCTTTAAC TATTTGCATC TATGCAGCTT TTACTTCCAC CTCCTCAACC CACTGTCTAC
AATTCTCACA
TAGAATTAAG AATAATTTTG CATAGCAGAT AATTGGCTGA GCATGCGGTA TTCTTTTGAC CCATTCAAGT
GAATAAAATA
CTGTATAGGA ACACTGTCAC AATTTAAATG AAATTAAATT TCTTGCCCCT TTTCCTCCCC CGACCATTTA
GTCTTTGGGT
AGCAACAGAG ATCACTAACA TGATATAACA ATTAATGTAG TTTTGTTTCA GGGCATTAAT TTGATACAAA
TTGTAATTCT
GTTCTCATCA GTTCTGTAAA
Y
TGCTTTACTG TAATTACACC AAGTATTTGA TCAAATATTG CCGATATTTC CATCTGTTTA GTGTATGGTA
TTATGATTTC
AAAATTGATT CTAGATTTAG TCCAATAATT TTTAGAATGT CTCTTTCTAT ATAAAAGTCA ATGCAGAAAT
AATAATATTT
TGAGATAAAA AATAAAGGCA TCTTCTAGTT AATATAACAG ATTTTAGTCA CATTTATATT CTTTAATGTT
CAGTGTATGT
ATCCACTACA TGAGAAACTC AAGACACAAA CAAAATGGTT ATATTTACTG CAACACTAAG CAATATGGAA
CATTAAAAAG
AATATGTATT CATAAACAAG ACAAAAGATG GTATAGCAAC ATAAAATTTA CAAGAACATT ATAGCTGGAC
TGTATAGGAA
GAGCTTGACT GATTTCTTTC CACAATGCAT TTTCATCAAA ACTCATACAT ATTCAGAGAT ACAAATAGCA
TACCAGTGAA
TTCAATGAAC TGCCACTGAA
>gnl|dbSNP|rs9466290
rs = 9466290|pos = 501|len = 1001|taxid = 9606|mol = “genomic”|class = 1|
alleles = “A/G”|build = 137|suspect = ?|GMAF = A: 2184:0.1589
CCAGTGTATG AATTTGGAGG GGAGATAAGC ATTCAGTCCA TTGCACCCTT ATTTCCAAGG CCCAGGGATA
ACGCTGAGCT
CCTCTGTGGG TGAAGCACAT TCAGCTATAA AACAGTATCT TAAGATTTTC TTCTCGAGTT AGATTTGGTA
CGTAGATAAC
GACCTTTAAC TATTTGCATC TATGCAGCTT TTACTTCCAC CTCCTCAACC CACTGTCTAC AATTCTCACA
TAGAATTAAG
AATAATTTTG CATAGCAGAT AATTGGCTGA GCATGCGGTA TTCTTTTGAC CCATTCAAGT GAATAAAATA
CTGTATAGGA
ACACTGTCAC AATTTAAATG AAATTAAATT TCTTGCCCCT TTTCCTCCCC CGACCATTTA GTCTTTGGGT
AGCAACAGAG
ATCACTAACA TGATATAACA ATTAATGTAG TTTTGTTTCA GGGCATTAAT TTGATACAAA TTGTAATTCT
GTTCTCATCA
GTTCTGTAAA TTGCTTTACT
R
TAATTACACC AAGTATTTGA TCAAATATTG CCGATATTTC CATCTGTTTA GTGTATGGTA TTATGATTTC
AAAATTGATT
CTAGATTTAG TCCAATAATT TTTAGAATGT CTCTTTCTAT ATAAAAGTCA ATGCAGAAAT AATAATATTT
TGAGATAAAA
AATAAAGGCA TCTTCTAGTT AATATAACAG ATTTTAGTCA CATTTATATT CTTTAATGTT CAGTGTATGT
ATCCACTACA
TGAGAAACTC AAGACACAAA CAAAATGGTT ATATTTACTG CAACACTAAG CAATATGGAA CATTAAAAAG
AATATGTATT
CATAAACAAG ACAAAAGATG GTATAGCAAC ATAAAATTTA CAAGAACATT ATAGCTGGAC TGTATAGGAA
GAGCTTGACT
GATTTCTTTC CACAATGCAT TTTCATCAAA ACTCATACAT ATTCAGAGAT ACAAATAGCA TACCAGTGAA
TTCAATGAAC
TGCCACTGAA ACCAAAACAT
>gnl|dbSNP|rs12175352
rs = 12175352|pos = 501|len = 1001|taxid = 9606|mol = “genomic”|class = 1|
alleles = “C/T”|build = 137|suspect = ?|GMAF = C: 2184:0.2042
AGAATTGTGT TTTACTGTAG TATACCATAC CTTATATGTT TAATAGAACA TATCTCATCT ATATTTTGTA
ATTCATTTTT
ATAAAACTGC CTGTAAATAG AAAAATTTTA TTATCTCTTT CATGTCTACA GCTTCTACAT TTATGTCCCC
TCTTACTTTT
TTTTTTTTTG GAGAGACAGT GTCTCACTAT GTTGCCTAGA CCAGTTTCAA ACTCCTGGGC TCAAGTGATC
CTTCTGCCTC
AGCCTCCCAA AGCGTTGGAA CTACAGGTGT GAGCCAGCCC GCCTGGCCCC TCTTACATTC TTTGTTGTTG
TTGTTTTGTT
GTTGTTGTCT TTGTTGTTTT TTGAAGCAGA GTCTCCCTCT GTGGCCCAGG CTGGAGTGCA GTGGCTTGAT
CGTGGCTCAC
TGCAACCTCC GCCTCCCAGG TTCAAGCCAT TCTCCTGCCT CAGCTTCCCG AGTAGCTAGG ACTGTAGGCA
TATGCCACCA
CGCCCAGCTA ATTTTTTTTA
Y
AATTTTAGTA GAGATGGGTT TTCACCATGT TGGCCAGGCT GGCCTCAAGC TCCTGACCTC CAGTGATCTT
CCTGCCTTGG
CCTCCCAAAA TGCTGGGATT ACAGGCATGA GCCACTGGGC CGAGCCCCCT TACATTCTTA ATATAGTTTA
TTTGTGTCCT
TTCTTTTTTC ACTTTTTCAC TCTTGCTTAC AGGTTTTTCA ATTGTGTTAG GCTTTTTAAA GAACTGCTTG
TCTTTGTAAT
GCTCTATATT ATAAATTTTA GTTGTATTTA TTTACTTCTG CTCTTACATT AATTTTTCAT TTTTGATCTT
TGAATTTATT
TTGCTGTTGT TTTCTAATTC TAATTTGGAT GACTAGCTAA TTAAAAAAAA TGTTTTCCCA CTTCTTAAAT
GTCAGCATTT
ATAGGCTTTT GCGCTATACA TTTCACTCAA AGTAGATTTT GTGAAATGCC ACAATATTTG GCATGTGGCA
TTTTCACAAT
TGTCAATTCA AAATGTTTTC
>gnl|dbSNP|rs12526269
rs = 12526269|pos = 501|len = 1001|taxid = 9606|mol = “genomic”|class = 1|
alleles = “A/T”|build = 137|suspect = ?|GMAF = A: 2184:0.2015
AGCTGTGATA TTGTGAATAT AAGAATTGTA CAATTTTCCT TTGAAATGTG CTGCTGAGAA GAATAAAAAT
GAAGTTTTCT
CTCTGGAAAT GGCTGGAAAC AAACGTCAAA ACCTGACAAT TTACACACAC AGTTCTCTTT CTGACTTGAC
TCACCCCTTT
GAGAATTACT TTTTAAAACA CCAAAGTTAT AGGATACTAA GTTAATTGTG GTCTTTCTAA TTCTGAAAAA
CTGGTTTTCA
TTTGCCTCAA GAACTTTTAA GGCAAAGTTG TCTAAGATAC TTCCTTGAAC AAAGACTCAG ACAAAAGCAT
TTGACTGCTT
TTAATTTCTC AGCATTTTTA CATTTTAAAA CTATAGCTTG AATGAAACTC AAGTGTCCTG AATAAAGAAT
AAATACTTAA
AAATTGTTTA AATACATATT TTCTCCTTTC ATTGTTGGAG CATTCAAGCA AAGATTGTGT AAAATTCAGG
TTAAGTAAAA
TGTAAAAAAT ACATATCCAG
W
TTACATTACA TATTCTTTTT GTGTACAAAT ATTTATACCA ATAAAAACCC CCTAAGATAT TTATATCTTT
AACATCTATT
TTTCTTTTAC CTTTACTACT AGAAAGAGAA GCTAACAAAG GAAAGCCTCT TCAAAAAATG GGATTTCCTT
GGCCTTAGCA
GTTCTGGTGT GTTCCACTGC CAACACTAGG TAGGAAAAAT CTGACTTGTG ATGTTGTGAT TAAATAGCGG
CCTGGCTCAA
ACTGCCCAAA GAGAGGGAAT TCACTATGAG TTCCACAGTT TTATCTTGAG GAAGAAACTA GCAGAAACAC
AGAATTTTAG
AGCCTTAGAG CTCTCATTAC AAAATGGCCC TCACTATAAA GTGGCATATA CTGAACCCAG CTTAGATGTG
TGTACTGAGC
CCAGTACAAA TGTATATAGT GAACCCAGAG CTCTCATTAC AAAATGCCTC TCACTATAAA GTGGCATATA
CTGAACCCAG
CTTAGGTGTG TGTACTGAGC
>gnl|dbSNP|rs35076407
rs = 35076407|pos = 501|len = 1001|taxid = 9606|mol = “genomic”|class = 1|
alleles = “C/T”|build = 137|suspect = ?|GMAF = C: 2184:0.4986
TTGAGCCCAG GAGTTCAAGA CCAGCCTGGG CAACAAGGCC AGACCCCATC CCTACAAAAA ATTTTTTTAA
AAAAATAGCC
ACCAGGTATG GTGGTGCACG CCTGTGGTCT CAACTGCTTG GGAGGCTGAG GCAGGAGGAA TGTTTGAGCC
CAGAAGGTCG
GGGCTGCAGC AGTGAGCTGT GATCACACCA CTGCATTCCA GCTTGGGTAA CAGAGTGAGA TCTTTTCTCA
AACAAACAAA
CAAACAAACA AAAAAAAGAT TTGGAATCAA TATCCTAGCA AGACTCTGGG TTGCAACTTT GCAAATCTTC
TGCTGTGCAC
GTTTGTTGTT GTTGTTGAGA CACAGTCTCG CTCTGCTGCC CAGGCTGGAG TGCAGTGGCA CAATCATTGC
TCACTGAAAC
CTCGACCTCC TGGACTCAAG CATTCCTCCC GCGTCAGCCT CCCAAGTCTC TGGGACTATA GGCGTGCACC
ACCACGCCTG
GCTAATTAAA TAAAAAATTG
Y
GGGTGCCAGG CGCGGTGGCT CACGCCTATA ATCCCAGCAC TTTGGGAGGG CGAGGAGGGT GGATCACGAG
GTCAAGAGAT
TGAGACCATT CTGGCCAACC TGGTGAAATC CAGTCTCTAC TAAAATTACA AAAATTAGCC GGGCGTGGTG
GCGCATGCCT
GTAGTCCCGG CTACTCGGGA GGCTGAGGCA GGAGAATCAC TTGAAGCCGG GAGGCAGAGG TTGCAGTGAG
CCGATATTGT
ACCACTGCAC TCCAGCCTGG CGACAGAGCA AGACTTCGTT TCAGAAAAAG AAAAAAAATT TTTTTTTTTT
GTAGAAACAG
AGTCTTTCTA TGTTGCCCAG GCTGATCGCA AACTCCTGGG CTCAAGGGAT CCTCTCACCT CCCAAAGTGC
TGGGATTACA
GGCCTGAGCC ACCTTCCCCA GCCCTATGCA CATTTTCACA AAGATATTTT GAACCCTAGC AGTGGGAAAG
GATACTTTTT
GACTGGTGAC ATCCCAGAGC
>gnl|dbSNP|rs56365413
rs = 56365413|pos = 501|len = 1001|taxid = 9606|mol = “genomic”|class = 1|
alleles = “C/T”|build = 137|suspect = ?|GMAF = T: 2184:0.1598
TAATGTAACA GGTAGGAAAC ACTCAATATT CGGGAATGCT GATGAGTTTC CTGAGTAGCA TGCGACTGGA
ATGGGAAGAA
CAGGAATCTG GTATCAGCCT AGCCACAGGC TGGCTGTTAT GTCCTTGGGC AAGTTTCATC TTGTCTCTGG
GTTGTATTTC
CTGACCTGTA ATGCAAGAGT ATCAGTCCCA ACCATCTCTG AGGCCCTTTC CAGCTCTCAG AGACTCTGGA
ATCAGATTCA
TACATCTGTC AGCTGAGTTT CCAAACAACA CAGCCTGGAA ACAACAATTC TAAAAATAAA ACATACAACT
AAGAAACCAG
TCACAAGACT AGAAAACATC ATCACATGTA TTCTGTCACT GGTAACAAAA TAGTTGCATA CATATGCGAG
CCAGTCCTAT
TTATTATTAT TTTTTTTTTA GAGACAGGGT CTTGCTCTAT TGCCCAGGCT GGAGTGCAGT CACCGCTAAC
TGCAGTCTTG
AACTCCTGTG CTCCAACGAT
Y
CTCCTGCCTC AGCTTCCCAA GTAGCTGGGA CCACAGGTGA ACATTGCCAC ACCTGGCTAA TTTTTTATTC
TTATTTTTAT
TAGAGAAGAG TTCTCCCTTT GTTGCCGAGG CTGGTCTCAA ACTCCTGGTT TCAAGTAATC CTACTGCCTC
AGCCTCCCAA
AGTGTTGGGG TTACAAGCAC GAGCCACTAT GCCCAGCCAA GTCAATCCTA TTTTAGATTC ATCATTATTC
ATTAGAAAAA
GTATTTTCAC TTATTTGTAT CTCACCCTAA CTTATAAAAT CCAAATAACG TTTGACTGAC ATGAAGATAA
TATGCCACTT
AATCATGTTA TTGACTTCAC TTAAGTGGGC TACACAAATA ACGCCAATAA TATTCAGTAA TATAAAAATT
ATGACATTTT
TCTAAGGGAA CTGAGTCATA CAGGCCTTGA ATGATTATAT ATAATAATCT TTTTTTTTTT TTTTTTTTTT
GGACACGGAG
TTTTGCTCTT GTTGCCCAGG
>gnl|dbSNP|rs75769093
rs = 75769093|pos = 501|len = 1001|taxid = 9606|mol = “genomic”|class = 1|
alleles = “A/C”|build = 137|suspect = ?|GMAF = C: 2184:0.429
TCCCATCACT CAGATAGTGA GCATAATTAT ACTGTATACA GTTTAGGAAA TAATAACAAG AAAAAAGTCT
ATACATATTC
CATACAGACA TGATCATCCT TTCCTCCCAC CCCTTGATTT TTTATTGAAT TCATGAATAT TTGCTGAGTC
CATGAATGTG
GAACCCACGG ATAGGGAAGG CTGACTGCAC CTACATTATG ACCTGGCAAT TCCACACCTA GGTTACTCAC
CCGGGAGAAA
TAAAAGCATA TGTCCTCAAA GAGGCTTGTT CAAAAATGTC CATAGCTTTA TTCATAAATA ACTGAAAGCT
GAAAACAACC
AATAGGAGAA TGAATAAACT AACTGTGGTA AATTCAGACA ATGAAATACT ACACAATAAA AAAGGGAGGA
ACCGGCTGGG
CGCGGTGGCT CACACCTGTA ATCCCAGCAC ATTGGGAGGC CGAGGTGGGT GGATCACCTG GGGTCAAGAG
TTCGAGACCA
GCCTGGCCAA CATGGTGAAA
M
CCCATCTCTA CTAAAAATAC AAAAATAGTC AGGTGTGGTG GCACGCACCT CCAATCCCAG CTACTCGAAA
GGCTGAGGCA
GGAGAATCAG CTTGAATCCA GGAGGCAGAG GTTGCAGTGA GCTGAGATTG TGCCACTGCA CTCCAGCCTG
GGTGACTCTG
TCTCAAAAAA AAAGGGGGGG TGGGGGGAGG AACCATTGAT ACATACAACA TCATGATGAA TTCCAAAAAT
GTTGTACTGA
ATGGAAGAAG CCTTACACAA GAAAGCACAT ACTGTTTATA TATTTATCAT CCTAGAACAA GCAAAACTAA
TCTATGGACT
AATCTAAGGT GGGGGCAGGG GAATCCAGAG AAAGGGTTAC CTTTGGGAGT TGGGCCATGG GGTTGGGGAC
CAGCTGTGCA
GGGGAGCTTT CAGGGCAGTC TTAAGGTACT GGATCTCACC AGGGGTTTGC CTTGCTTGTA TACATGGCCT
TCCTTGCAAC
TTGCTGAATG GCACACTTAT
>gnl|dbSNP|rs78113724
rs = 78113724|pos = 501|len = 1001|taxid = 9606|mol = “genomic”|class = 1|
alleles = “A/C/G/T”|build = 137|suspect = ?|GMAF = A: 2184:0.2779
TTGCAAACAT TTATTTTCTC ACAGTTCCGG AGGCTGGAAG TCCAAGATGG AGCTGCTGTG AGGGTTGGTT
TCCGGTGAGG
CCTTTCTTCC TAGCTCGTAG ACAGCCACCT TCTCTCTGTG TCCTCACATG GCTTTTCTTT TGTGTCCATG
CCGAGAGAGA
GGACTCTCTG AGGACTCTCC CTCTTCTTGT AAGGACACCA GTCCTATCAG ACTAGGGCCC CACTCTTATG
ACCTCATTTA
ATTTAATTAT GTCTGTAAAG GCCCCTGCTC CAAATATAGT CACATTGAGG ATTATGGCTT CATAATCCTG
TGACTCTGGG
GAGAGGACAC ATTTCAGTCC ATAACAAAGC CCTTAGTGTG TTTTAGTGCT CAAAACTGTT CATTCATACC
TCGGTTATTC
CATTATTATT GCCTACGATA TTACCACTTC AGGGTTTTTG TTATTTTTTA CAATATAGAG CACAACGTAT
AATAAACTAC
ACATACGAAT TCTCATTGAG
N
AATTACAGAA AATATATCTA TCGCGTCTAA CAAGGTTTAA TTAGCATCTT GGAAAAAAAA AAAAACACCT
ACGTTTTTAA
GGAAAAAGTT GGCCAATACT GCCATCTGTT GGAATTTTGG TCAAAGCTCA TGTGTTGGAC TTTACTCATT
CTTTGTCAAT
ATCTTTTCTT TCTTTCTTTC TTTCTTTTTT TCTGAGACGG AGCCTTGCTC TGTTACCCAG GCTGGAGTGT
GGTGGCGCGA
TCTCGGCTCA CTGCAACCTC CGCCTCCTAG GTTCAAGCGA TTCTCCTGCC TTGGCCTCCT GAGTAGCTGG
AATTACAGGC
ACGCGCCACC ACGCCCGGCT AATTTTTGTA TTTTTAGTAG AGACGGAGTT TCACCATGTT GGTCAGGCTG
GTTTCGAACT
CCTGACCTCG TGATCCACCC ACCTCGGCCT CCCAAAGTGC TGGAATTACA GGCGTGAGCC ACCGCGCCCG
GCCCTTTGTC
AACATCTTAT ATGTTGCTGT
>gnl|dbSNP|rs115262601
rs = 115262601|pos = 201|len = 401|taxid = 9606|mol = “genomic”|class = 1|
alleles = “A/C”|build = 132|suspect = ?|GMAF = C: 2184:0.006
GTGAGAGACA GAGTCACAAA GAGAAAGAGA CAGTGAGGGG CCAGAACGAC TCTCTTTTCT CCGATTGTCA
ATGCCCAGTG
GGAGCCGGGA GCCCAACAGG CCCAGCCCAT CAGATTCGGC CCCTCCGGGC CCCAAATCCG CTCGCCCCAC
CCGAGATCCA
GGCCTCCAGC CACTTGCCTA ACTGTGAGCC CGCAAGAGCC
M
GGCCCGCGGC TCCCTCCTTC CTCCTCCTGC GGCAGTCTCG CGGCTTTCAA ACCTTAGTCG AACCCACAGA
AGGCCCAGTC
CCAGGCCAAA CCTACTCAAC AGGCACCTTC TCACGGCCTA GGAATTCTGC AGCGAAATTC ACTGGAATTT
GAGGAGAAAA
CCCAAAGACT GCTCCGAAAG GACTCCCCCA GTCTTCAGCC
>gnl|dbSNP|rs184577|allelePos = 501|totalLen = 1001|taxid =
9606|snpclass = 1|alleles = ‘C/T’|mol = Genomic|build = 137
CTATGGAAGT TTCTCAAAAA ATTAAAAATA GAACTACCAT GTGATCCAGA AATCCCACTG
CTGGGTATTT ATCCAAAGGA AAAAAAATCA ATATATCAAA GGGAGACCTG CACTCCCATG
TTTATTGCAG CACTATTCAC AATAGCCAAG ATACAGTATC AATCTAAGTG TCCATCAACA
GATGAATGGA TAAAGCAAAT GTGATACACA CACACACACA CACACACACA CACACACACA
ATGGAATACT ATTAAGCCGT AAAAAAGAAT GAAATTCTAT CATTTGCAGG AACATGTATG
GAATTGAAGG GCATCTTGTT AAGTAAAATC AGCCAGGCAC CGAAAGACAA ATATTGCATA
TTCTTACTCA TATGTGGGAG CTAAAAAGAT GGATCTCATG GAGGTAGAGA AAAGAATGGT
AGCTACTAGA GGCTATGAAG GGTGTGTGGG ATGAAGAGAG GTTGGTTAAT AGGTACAGAC
ATATAGTTAG ACGGAATAAG
Y
GCTAGTATTC AGCCTCAAAG TAGGGTGACT ATAGTTAACA AAAACATATT GAGTATCTCA
AAATAGCCAG AAGAGAAAAT TTGAAATGTT CCTAGCTCAA AGAAATGATA CATGTTCAAG
GCGATGGATA TCCTAAATAC CCTGATTTGA TCATTACACA TTCTATGGCT GTGTCAAGAT
ATCACAGATA CCCCATAAAT ATGTGTAATT ATTATGTATC AAAAACTTTA TATAAAAAAC
ATTAATTTGC TGTATTTTTG ATTCTACAAT TGGGCAGCAC TTTATTCCAT AAAATAGAAT
GAGTGTTCTG ATGAGCCAAG AAGAGGAGGT TGGTTTTACA GACAGAAAAG GGCTGTGGAA
AGCAGAAAAA AACAAACAAA AAAAATGTGG ATTGGTCATT TCAAAGTTTC TTTTCATGTA
AAGGTTAAAG CAGAGGGGAC TTTCTTGTCC TGCTGGCACT GGATCTAGGT CAGTGTTGGA
GACGATGTCT GGGACTCAGG
>gnl|dbSNP|rs619373|allelePos = 61|totalLen = 121|taxid =
9606|snpclass = 1|alleles = ‘A/G’|mol = Genomic|build = 137
TTTGGGGGCT GAGTGTTCTT CCAATCCTAA AAACAACTCT CTGTGGCCAA ATTGCTCCAG
R
CCTCCAACAT ATCCAACATA GCTATATGTG AATAGAGTCA TCAGCTTCTG CTGTTCGTAT
ADDITIONAL NOTES ABOUT TABLE 7:
Global Minor Allele Frequency (GMAF) [ie. G: 0.262:330 <-- (allele:count:frequency)]
(http://www.ncbi.nlm.nih.gov/projects/SNP/docs/rs_attributes.html#gmaf)
“G: 0.262:330”. This means that for this rs, minor allele is ‘G’ and has a frequency of 26.2% in the 1000Genome phase 1 population and that ‘G’ is observed 330 times in the sample population of
629 people (or 1258 chromosomes).

### 7. REFERENCES

1. Antoniou A C, Cunningham A P, Peto J, Evans D G, Lalloo F, et al. (2008) The BOADICEA model of genetic susceptibility to breast and ovarian cancers: updates and extensions. Br J Cancer 98: 1457-1466.
2. Gaudet M M, Kirchhoff T, Green T, Vijai J, Korn J M, et al. (2010) Common genetic variants and modification of penetrance of BRCA2-associated breast cancer. PLoS Genet 6: e1001183.
3. Turnbull C, Ahmed S, Morrison J, Pernet D, Renwick A, et al. (2010) Genome-wide association study identifies five new breast cancer susceptibility loci. Nat Genet 42: 504-507.
4. Lindstrom S, Vachon C M, Li J, Varghese J, Thompson D, et al. (2011) Common variants in ZNF365 are associated with both mammographic density and breast cancer risk. Nat Genet 43: 185-187.
5. Couch F J, Gaudet M M, Antoniou A C, Ramus Si, Kuchenbaecker K B, et al. (2012) Common variants at the 19p13.1 and ZNF365 loci are associated with ER subtypes of breast cancer and ovarian cancer risk in BRCA1 and BRCA2 mutation carriers. Cancer Epidemiol Biomarkers Prev 21: 645-657.
6. (2006) Commonly studied single-nucleotide polymorphisms and breast cancer: results from the Breast Cancer Association Consortium. J Natl Cancer Inst 98: 1382-1396.
7. Gayther S A, Song H, Ramus S J, Kjaer S K, Whittemore A S, et al. (2007) Tagging single nucleotide polymorphisms in cell cycle control genes and susceptibility to invasive epithelial ovarian cancer. Cancer Res 67: 3027-3035.
8. Kote-Jarai Z, Easton D F, Stanford J L, Ostrander E A, Schleutker J, et al. (2008) Multiple novel prostate cancer predisposition loci confirmed by an international study: the PRACTICAL Consortium. Cancer Epidemiol Biomarkers Prev 17: 2052-2061.
9. Kermani B G (2008) Artificial intelligence and global normalization methods for genotype.
10. Robertson A, Hill W G (1984) Deviations from Hardy-Weinberg proportions: sampling variances and use in estimation of inbreeding coefficients. Genetics 107: 703-718.
11. (2010) A map of human genome variation from population-scale sequencing. Nature 467: 1061-1073.
12. Howie B N, Donnelly P, Marchini J (2009) A flexible and accurate genotype imputation method for the next generation of genome-wide association studies. PLoS Genet 5: e1000529.
13. Couch F J, Wang X, McGuffog L, Lee A, Olswold C, et al. (2012) Genome-wide association study in BRCA1 mutation carriers identifies novel loci associated with breast and ovarian cancer risk. Nat Genet under review.
14. Michailidou K, Hall P, Gonzalez-Neira A, Ghoussaini M, Dennis J, et al. (2012) Large-scale genotyping identifies 38 new breast cancer susceptibility loci. Nat Genet under review.
15. Barnes D, Lee A, Embrace, Easton D, Antoniou A C (2012) Evaluation of association methods for analyzing modifiers of disease risk in carriers of high-risk mutations. Genet Epidemiol in press.
16. Antoniou A C, Goldgar D E, Andrieu N, Chang-Claude J, Brohet R, et al. (2005) A weighted cohort approach for analysing factors modifying disease risks in carriers of high-risk susceptibility genes. Genet Epidemiol 29: 1-11.
17. Antoniou A C, Sinilnikova O M, Simard J, Leone M, Dumont M, et al. (2007) RAD51 135G-->C modifies breast cancer risk among BRCA2 mutation carriers: results from a combined analysis of 19 studies. Am J Hum Genet 81: 1186-1200.
18. Antoniou A C, Wang X, Fredericksen Z S, McGuffog L, Tarrell R, et al. (2010) A locus on 19p13 modifies risk of breast cancer in BRCA1 mutation carriers and is associated with hormone receptor-negative breast cancer in the general population. Nat Genet 42: 885-892.
19. Mulligan A C, Couch F J, Barrowdale D, Domehek S M, Eccles D, et al. (2011) Common breast cancer susceptibility alleles are associated with tumour subtypes in BRCA1 and BRCA2 mutation carriers: results from the Consortium of Investigators of Modifiers of BRCA1/2. Breast Cancer Res 13: R110.
20. Aulchenko Y S, Ripke S, Isaacs A, van Duijn C M (2007) GenABEL: an R library for genome-wide association analysis. Bioinformatics 23: 1294-1296.
21. Lange K, Weeks D, Boehnke M (1988) Programs for Pedigree Analysis: MENDEL,
FISHER, and dGENE. Genet Epidemiol 5: 471-472.
22. Liu J Z, McRae A F, Nyholt D R, Medland S E, Wray N R, et al. (2010) A versatile gene-based test for genome-wide association studies. Am J Hum Genet 87: 139-145.
23. Ashburner M, Ball C A, Blake J A, Botstein D, Butler H, et al. (2000) Gene ontology: tool for the unification of biology. The Gene Ontology Consortium. Nat Genet 25: 25-29.
24. Antoniou A C, Beesley J, McGuffog L, Sinilnikova O M, Healey S, et al. (2010) Common breast cancer susceptibility alleles and the risk of breast cancer for BRCA1 and BRCA2 mutation carriers: implications for risk prediction. Cancer Res 70: 9742-9754.
25. Antoniou A C, Kuchenbaecker K B, Soucy P, Beesley J, Chen X, et al. (2012) Common variants at 12p11, 1424, 9p21, 9q31.2 and in ZNF365 are associated with breast cancer risk for BRCA1 and/or BRCA2 mutation carriers. Breast Cancer Res 14: R33.
26. Friedrichs N, Jager R, Paggen E, Rudlowski C, Merkelbach-Bruse S, et al. (2005) Distinct spatial expression patterns of AP-2alpha and AP-2gamma in non-neoplastic human breast and breast cancer. Mod Pathol 18: 431-438.
27. Gee J M, Robertson J F, Ellis 10, Nicholson R I, Hurst H C (1999) Immunohistochemical analysis reveals a tumour suppressor-like role for the transcription factor AP-2 in invasive breast cancer. J Pathol 189: 514-520.
28. Gaubatz S, Imhof A, Dosch R, Werner O, Mitchell P, et al. (1995) Transcriptional activation by Myc is under negative control by the transcription factor AP-2. EMBO J 14: 1508-1519.
29. McPherson L A, Loktev A V, Weigel R J (2002) Tumor suppressor activity of AP2alpha mediated through a direct interaction with p53. J Biol Chem 277: 45028-45033.
30. Zhang H, Meng F, Liu G, Zhang B, Zhu J, et al. (2011) Forkhead transcription factor foxq1 promotes epithelial-mesenchymal transition and breast cancer metastasis. Cancer Res 71: 1292-1301.
31. Zhang H, Meng F, Wu S, Kreike B, Sethi S, et al. (2011) Engagement of I-branching {beta}-1,6-N-acetylglucosaminyltransferase 2 in breast cancer metastasis and TGF-{beta} signaling. Cancer Res 71: 4846-4856.
32. Antoniou A C, Spurdle A B, Sinilnikova O M, Healey S, Pooley K A, et al. (2008) Common breast cancer-predisposition alleles are associated with breast cancer risk in BRCA1 and BRCA2 mutation carriers. Am J Hum Genet 82: 937-948.
33. Antoniou A C, Sinilnikova O M, McGuffog L, Healey S, Nevanlinna H, et al. (2009) Common variants in LSP1, 2q35 and 8q24 and breast cancer risk for BRCA1 and BRCA2 mutation carriers. Hum Mol Genet 18: 4442-4456.
34. Antoniou A C, Kuchenbaecker K B, Soucy P, Beesley J, Chen X, et al. (2012) Common variants at 12p11, 12q24, 9p21, 9q31.2 and in ZNF365 are associated with breast cancer risk for BRCA1 and/or BRCA2 mutation carriers. Breast Cancer Res 14: R33.
35. Antoniou A C, Kartsonaki C, Sinilnikova O M, Soucy P, McGuffog L, et al. (2011) Common alleles at 6q25.1 and 1p11.2 are associated with breast cancer risk for BRCA1 and BRCA2 mutation carriers. Hum Mol Genet 20: 3304-3321.

Various references are cited herein, the contents of which are hereby incorporated by reference in their entireties.

What is claimed is:

1. A kit for determining whether a subject has an increased risk of having or developing breast cancer comprising a means for detecting a 6p24 SNP biomarker.

2. The kit according to claim 1 wherein the 6p24 SNP biomarker is rs9348512 SNP.

3. The kit according to claim 1 further comprising a means for detecting a biomarker of BRCA2.

4. The kit according to claim 1 further comprising a means for detecting one or more biomarkers selected from the group consisting of 10q26 (FGFR2), 16q12 (TOX3), 12p11 (PTHLH), 5q11 (MAP3K1), 9p21 (CDKN2A/B), 11p15 (LSP1), 8q24, 20q13, 6q25 (ESR1), 10q21 (ZNF365), 3p24 (SLC4A7, NEK10), 12q24, 5p12 and 11q13.

5. The kit according to claim 1 further comprising a means for detecting one or more biomarkers selected from the group consisting of:
(a) a biomarker of 10q26 (FGFR2) which is the SNP rs2420946;
(b) a biomarker of 16q12 (TOX3) which is the SNP rs3803662;
(c) a biomarker of 12p11 (PTHLH) which is the SNP rs27633;
(d) a biomarker of 5q11 (MAP3K1) which is the SNP rs16886113;
(e) a biomarker of 10q26 (CDKN2A/B) which is the SNP rs10965163;
(f) a biomarker of 8q24 which is the SNP rs4733664;
(g) a biomarker of 6q25 (ESR1) which is the SNP rs2253407; and
(h) a biomarker of 10q21 (ZNF365) which is the SNP rs17221319.

6. A method for assessing the likelihood that a subject has or will develop breast cancer comprising determining whether the subject carries a 6p24 SNP biomarker and a BRCA2 biomarker, where the presence of both biomarkers indicates that while the subject has an increased risk of having or developing breast cancer relative to the general population, the risk is less than if the 6p24 biomarker were absent.

7. The method of claim 6, wherein the subject has previously been tested and known to carry a BRCA2 biomarker.

8. The method of claim 6, wherein the 6p24 SNP biomarker is rs9348512 SNP.

9. The method of claim 6, further comprising determining whether the subject carries one or more auxiliary biomarkers selected from the group consisting of 10q26 (FGFR2), 16q12 (TOX3), 12p11 (PTHLH), 5q11 (MAP3K1), 9p21 (CDKN2A/B), 11p15 (LSP1), 8q24, 6q25 (ESR1), 10q21 (ZNF365), 3p24 (SLC4A7, NEK10), 12q24, 5p12, and 11q13.

10. The method of claim 6, wherein the presence of the biomarker is determined in a sample taken from the subject.

11. The method of claim 6 comprising the further step, where the subject is found to carry a 6p24 SNP, of recommending or performing regular breast screening to monitor for the presence of cancer.

12. The method of claim 11 wherein screening is performed by a clinical breast exam, biopsy, mammography, ultrasound, magnetic resonance imaging, or similar techniques.

13. The method of claim 6 comprising the further step, where the subject is found not to carry the 6p24 SNP, of recommending or performing a mastectomy or oophorectomy, or recommending or administering anti-estrogen therapy or chemoprevention.

14. A method of treating a subject who carries a BRCA2 biomarker, comprising determining whether the subject carries a 6p24 SNP biomarker and, where the 6p24 SNP biomarker is absent, advising the subject that she is at high risk for developing breast cancer relative to a subject carrying both the 6p24 SNP and BRCA2 biomarkers and to the general population.

15. The method of claim 14, wherein the subject has previously been tested and known to carry a BRCA2 biomarker.

16. The method of claim 14, wherein the 6p24 SNP biomarker is rs9348512 SNP.

17. The method of claim 14, further comprising determining whether the subject carries one or more auxiliary biomarkers selected from the group consisting of 10q26 (FGFR2), 16q12 (TOX3), 12p11 (PTHLH), 5q11 (MAP3K1), 9p21 (CDKN2A/B), 11p15 (LSP1), 8q24, 6q25 (ESR1), 10q21 (ZNF365), 3p24 (SLC4A7, NEK10), 12q24, 5p12 and 11q13.

18. The method of claim 14, wherein the presence of the biomarker is determined in a sample taken from the subject.

19. The method of claim 14 comprising the further step, where the subject is found not to carry the 6p24 SNP, of recommending or performing a mastectomy or oophorectomy, or recommending or administering anti-estrogen therapy or chemoprevention.
